(19) United States
(12) Patent Application Publication
(10) Pub. No.: US 2024/0327512 A1
(43) Pub. Date: Oct. 3, 2024

(54) COMPOSITIONS AND METHODS FOR TREATMENT OF RETINAL DEGENERATION

(71) Applicants: Duke University, Durham, NC (US); Ludwig-Maximilians Universität München, Munich (DE); Deutsches Zentrum für Neurodegenerative Erkrankungen e. V., Bonn (DE)

(72) Inventors: Daniel Saban, Durham, NC (US); Chen Yu, Durham, NC (US); Christian Haass, Munich (DE); Kai Schlepckow, Munich (DE)

(21) Appl. No.: 18/597,372

(22) Filed: Mar. 6, 2024

Related U.S. Application Data
(63) Continuation-in-part of application No. PCT/US22/76078, filed on Sep. 7, 2022.
(60) Provisional application No. 63/241,125, filed on Sep. 7, 2021.

(51) Int. Cl. C07K 16/28; A61K 39/00; A61K 45/06; A61P 27/02; G01N 33/50; G01N 33/68

(52) U.S. Cl. CPC C07K 16/2803; A61K 45/06; A61P 27/02; G01N 33/5058; G01N 33/6854; A61K 2039/505; C07K 2317/34; C07K 2317/565; C07K 2317/76; G01N 2333/70503

(57) ABSTRACT

The present disclosure describes, in part, methods for treating a degenerative and vascular disease of the retina and/or posterior segment of the eye in a subject, the method comprising administering to a subject an effective amount of an antibody or fragment thereof that binds to the same epitope as an antibody comprising a heavy chain variable region and a light chain variable region, wherein: (a) the heavy the heavy chain variable region comprises a sequence having at least 85% sequence identity to SEQ ID NO: 4 and the light chain variable region comprises a sequence having at least 85% sequence identity to SEQ ID NO: 5 wherein the antibody inhibits TREM2 cleavage; or (b) the heavy the heavy chain variable region comprises a sequence having at least 85% sequence identity to SEQ ID NO: 6 and the light chain variable region comprises a sequence having at least 85% sequence identity to SEQ ID NO:7, and wherein the antibody inhibits TREM2 cleavage; or (c) the heavy the heavy chain variable region comprises a sequence having at least 85% sequence identity to SEQ ID NO: 8 and the light chain variable region comprises a sequence having at least 85% sequence identity to SEQ ID NO:9 or to SEQ ID NO:87, and wherein the antibody inhibits TREM2 cleavage; or (d) the heavy the heavy chain variable region comprises a sequence having at least 85% sequence identity to SEQ ID NO: 10 and the light chain variable region comprises a sequence having at least 85% sequence identity to SEQ ID NO:11 or to SEQ ID NO:12 or to SEQ ID NO:13 or to SEQ ID NO:14, and wherein the antibody inhibits TREM2 cleavage. and methods of screening for a molecule which specifically binds to and inhibits cleavage of human TREM2 between His157 and/or Ser158 on the surface of retinal microglial cells by ADAM10 and/or ADAM17.

### PRIORITY

This patent application is a continuation-in-part of International Patent Application No. PCT/US2022/076078 filed Sep. 7, 2022, which claims the benefit of US provisional application No. 63,241,125 filed Sep. 7, 2021, the contents of each of which are incorporated by reference herein in their entirety.

### FEDERAL FUNDING LEGEND

This invention was made with Government support under Federal Grant no. R01 EY030906 awarded by the National Institutes of Health. The Federal Government has certain rights to this invention.

### SEQUENCE LISTING

This application contains a Sequence Listing which has been submitted electronically as a WIPO Standard ST.26 XML file via Patent Center, created on Mar. 6, 2024, is entitled “123658-12001.xml” and is 102 KB in size. The sequence listing is incorporated herein by reference in its entirety.

### BACKGROUND

Retinal Degenerative Diseases (RDD) are a group of retinal diseases in which loss of retinal neurons occurs, and includes retinitis pigmentosa (RP), diabetic retinopathy (DR), Age-related Macular Degeneration (AMD), glaucoma, and Alzheimer's disease (AD)-related retinal degeneration (Yang et al., 2013; Madeira et al., 2015; Jin et al., 2019; Nashine et al., 2019), see Jin et al. Frontiers in Cell and Developmental Biology 13 Dec. 2021; Sec. Stem Cell Research (https://doi.org/10.3389/fcell.2021.741368). RDD generally result from the inability of the retina to detect and/or transmit light-triggered signals to the brain.

Approximately 200 million people suffer from AMD, yet barely 10% of these cases are treatable. The sole pharmacologic currently available is anti-vascular endothelial growth factor (VEGF) therapy, which works through the inhibition of sight threatening exudation in the retina brought in by neovessels in the choroid (the tissue that sits posterior to the retinal pigment epithelium (RPE)). Hence, only the ‘wet’ (i.e., exudative) form AMD is treatable.

By contrast, there is no pharmacological therapy available for patients suffering from the ‘dry’ (i.e., nonexudative) form, the type of AMD that most patients suffer from. With no pharmacological therapy available for dry AMD, most of the experimental drugs in clinical trials aim to target this form of the disease. Mechanistically, nearly all these experimental drugs are designed to target the complement cascade, the basis of which come from gene variants associated with AMD. However, most of these clinical trials have either failed (e.g., Lampalizumab, Eculizumab, Tesidolumab, CLG561) or have intolerable ocular side effects like conversion from dry to wet AMD (e.g. Pegcetoplan, Avacincaptad).

Retinal microglia cells function as tissue macrophages and play important roles in retinal homeostasis (Langmann, 2007, Microglia Activation in Retinal Degeneration. J. Leukoc. Biol. 81 (6): 1345-1351), specifically, mediating phagocytosis and regulation of tissue inflammation.

### SUMMARY OF THE INVENTION

The present disclosure is based, in part, on the discovery by the inventors that targeting the neuroprotective microglia bolsters these microglial cells for the purposes of restricting disease progression with respect to degenerative and vascular diseases of the retina and posterior segment. Such diseases include a retinal disease that leads to photoreceptor loss or outer-retina degeneration, as well as those diseases involving retinal pigment epithelium (RPE) atrophy as well as photoreceptor degeneration.

Accordingly, one aspect of the disclosure relates to method for treating a degenerative and vascular disease of the retina and/or posterior segment of the eye in a subject, the method comprising administering to a subject an effective amount of an antibody or fragment thereof that binds to an epitope of GESESFEDAHV (SEQ ID NO:2) of SEQ ID NO. 1.

The disclosure also relates to a method for treating and/or preventing an eye disease in a subject, specifically degenerative and vascular diseases of the retina and posterior segment, the method comprising, consisting of, or consisting essentially of administering to a subject an effective amount of an antibody or fragment thereof that binds to the same epitope as an antibody selected from 13H3, 9D10, 7F12, 13F4, 14B3 and 14D3.

The disclosure also relates to a method for treating a degenerative and vascular disease of the retina and/or posterior segment of the eye in a subject, the method comprising administering to a subject an effective amount of an antibody or fragment thereof, wherein the antibody comprises a heavy chain variable region and a light chain variable region, wherein: (a) the heavy the heavy chain variable region comprises a sequence having at least 85% sequence identity to SEQ ID NO: 4 and wherein the heavy chain variable region comprises a CDR1 comprising positions 23-35 of SEQ ID NO:4, a CDR2 comprising positions 50-59 of SEQ ID NO:4, and a CDR3 comprising positions 99-107 of SEQ ID NO:4, and the light chain variable region comprises a sequence having at least 85% sequence identity to SEQ ID NO: 5 and wherein the light chain variable region comprises a CDR1 comprising positions 24-39 of SEQ ID NO:5, a CDR2 comprising positions 54-61 of SEQ ID NO:5, and a CDR3 comprising positions 94-102 of SEQ ID NO:5, and wherein the antibody inhibits TREM2 cleavage; or (b) the heavy the heavy chain variable region comprises a sequence having at least 85% sequence identity to SEQ ID NO: 6 and wherein the heavy chain variable region comprises a CDR1 comprising positions 33-45 of SEQ ID NO:6, a CDR2 comprising positions 50-59 of SEQ ID NO:6, and a CDR3 comprising positions 98-107 of SEQ ID NO:6, and the light chain variable region comprises a sequence having at least 85% sequence identity to SEQ ID NO:7 and wherein the light chain variable region comprises a CDR1 comprising positions 24-39 of SEQ ID NO:7, a CDR2 comprising positions 54-61 of SEQ ID NO:7, and a CDR3 comprising positions 94-101 of SEQ ID NO:7, and wherein the antibody inhibits TREM2 cleavage; or (c) the heavy the heavy chain variable region comprises a sequence having at least 85% sequence identity to SEQ ID NO: 8 and wherein the heavy chain variable region comprises a CDR1 comprising positions 23-35 of SEQ ID NO:8, a CDR2 comprising positions 50-59 of SEQ ID NO:8, and a CDR3 comprising positions 98-107 of SEQ ID NO:8, and the light chain variable region comprises a sequence having at least 85% sequence identity to SEQ ID NO:9 or to SEQ ID NO:87, wherein the light chain variable region comprises a CDR1 comprising positions 24-39 of SEQ ID NO:9, a CDR2 comprising positions 54-61 of SEQ ID NO:9, and a CDR3 comprising positions 94-102 of SEQ ID NO:9, or wherein the light chain variable region comprises a CDR1 comprising positions 24-39 of SEQ ID NO:87, a CDR2 comprising positions 54-61 of SEQ ID NO:87, and a CDR3 comprising positions 94-102 of SEQ ID NO:87, and wherein the antibody inhibits TREM2 cleavage; or (d) the heavy the heavy chain variable region comprises a sequence having at least 85% sequence identity to SEQ ID NO: 10 and wherein the heavy chain variable region comprises a CDR1 comprising positions 23-35 of SEQ ID NO:10, a CDR2 comprising positions 50-59 of SEQ ID NO:10, and a CDR3 comprising positions 99-107 of SEQ ID NO:10 or positions 98-107 of SEQ ID NO:10, and the light chain variable region comprises a sequence having at least 85% sequence identity to SEQ ID NO:11 or to SEQ ID NO:12 or to SEQ ID NO:13 or to SEQ ID NO:14, wherein the light chain variable region comprises a CDR1 comprising positions 24-39 of SEQ ID NO:11, a CDR2 comprising positions 54-61 of SEQ ID NO:11, and a CDR3 comprising positions 94-102 of SEQ ID NO:11, or wherein the light chain variable region comprises a CDR1 comprising positions 24-39 of SEQ ID NO:12, a CDR2 comprising positions 54-61 of SEQ ID NO:12, and a CDR3 comprising positions 94-102 of SEQ ID NO:12, or wherein the light chain variable region comprises a CDR1 comprising positions 24-39 of SEQ ID NO:13, a CDR2 comprising positions 54-61 of SEQ ID NO:13, and a CDR3 comprising positions 94-102 of SEQ ID NO:13, or wherein the light chain variable region comprises a CDR1 comprising positions 24-39 of SEQ ID NO:14, a CDR2 comprising positions 54-61 of SEQ ID NO:14, and a CDR3 comprising positions 94-102 of SEQ ID NO:14, and wherein the antibody inhibits TREM2 cleavage.

The antibody may comprise a full length antibody or an antigen-binding fragment thereof. In one embodiment, the antibody is capable of binding to a protein comprising the amino acid sequence of SEQ ID NO:1, or a fragment thereof. In certain embodiments, the antibody or antigen binding fragment thereof includes one or more of the following antibodies described herein: F12, 13F4, 9D10 and 14B3. In other embodiments, the antibody or antigen binding fragment thereof includes one or more of the following antibodies described in U.S. patent Ser. No. 10/941,200B2: 14D3, 14D8, 7A12, 8A11, and 10C3, hereby incorporated by reference.

Another aspect of the present disclosure provides a method of identifying an agent that binds to a specific portion of human TREM2 (Triggering Receptor Expressed on Myeloid cells-2), that portion being set forth in SEQ ID NO:1 (the amino acid sequence of Human TREM2 at positions 141-170) so as to reduce cleavage and release of TREM2 from the retinol microglial cell surface and thereby restrict disease progression for degenerative and vascular diseases of the retina and posterior segment.

Preferably, the agent binds human TREM2 on the surface of microglial cells in the retinal pigment epithelium, resulting in reduced shedding of TREM2 from the microglial cell surface and increased stabilization of Trem2 expression on microglial cell surface, thereby promoting for example, phospho-Syk signaling.

Another aspect of the disclosure relates to a method for identifying an agent that bolsters TREM2 mediated neuroprotective functions of microglia in the retinal pigment epithelium, as assessed for example, by the agent's ability to increase the level of mature membrane-bound full-length TREM2 in retinal microglial cells by shedding inhibition as evidenced by reduced levels of both soluble TREM2 (sTREM2) and the C-terminal fragment (CTF) of TREM2; to inhibit TREM2 shedding in retinal microglial cells with IC50s in the low nM range, to activate pSYK signaling in retinal microglial cells, and/or to boost liposome-mediated pSYK signaling in retinal microglial cells.

In some embodiments, degenerative and vascular diseases of the retina and posterior segment include but are not limited to: Outer Retinal Diseases, Age-Related Macular Degeneration (AMD), Choroidal Dystrophy, Idiopathic CNV, Chorioretinal Inflammatory Non-Infectious Diseases, Hypertensive Retinopathy, Retinopathy of Prematurity, Macular Telangiectasia, Optic Nerve Disorders and Retinal Ganglion cell, and Uveitis-associated degeneration.

Such diseases of the retina and posterior segment also include but are not limited to (i) an outer retinal disease for example, Retinitis pigmentosa (RP), Rod dystrophy or rod-cone dystrophy, Usher syndrome (USH), Bietti crystalline dystrophy (BCD), Batten disease, Bardet-Biedl syndrome (BBS), Alport syndrome, Leber congenital amaurosis (LCA) or early onset retinal dystrophy (EORD), Cone dystrophy, Cone-rod dystrophy (CORD), Achromatopsia, Congenital stationary night blindness (CSNB), Macula dystrophy, Stargardt's disease, Best disease, Pattern dystrophy, Sorsby fundus dystrophy, Doyne's honeycomb dystrophy, Choroideremia, X-linked retinoschisis (XLRS), Retinal vein occlusion, Macular hole, Myopic macular degeneration, Bull's eye maculopathy, Pseudoxanthoma elasticum (PXE), Macular oedema, Central serous retinopathy, Punctate inner choroidopathy, Charles Bonnet syndrome, Retinal vein occlusion, Central Serous Chorioretinopathy (CSCR), Cystoid Macular Edema, Degenerative Myopia, Diabetic Macular Edema (DME), Epiretinal Membrane, Macular Pucker, Pseudohole, Vitreomacular Traction Syndrome, Diabetic Retinopathy, (ii) an Age-related Macular Degeneration for example, Geographic Atrophy, Geographic Atrophy and Calcified Drusen, Hard Drusen, Non Geographic Atrophy, Pigment Epithelium Detachment, Soft Drusen, (iii) a Choroidal Dystrophy, for example, Gyrate atrophy, Posterior Polar Central, Annular, or Hemispheric Choroidal Dystrophy, Pathologic Myopia, Angioid Streaks, Central Serous Chorioretinopathy, (iv) an Idiopathic CNV, (v) a Chorioretinal Inflammatory Non-Infectious Disease, for example, Sarcoidosis, Sympathetic Ophthalmia, Vogt-Koyanagi-Harada Syndrome, Birdshot Retinochoroidopathy, Behçet Disease, Serpiginous Choroidopathy, White-Dot Syndromes, Acute Retinal Pigment Epitheliitis, Acute Posterior Multifocal Placoid Pigment Epitheliopathy, (vi) Hypertensive Retinopathy, (vii) Retinopathy of Prematurity, (viii) Macular Telangiectasia, (ix) Optic Nerve Disorders and Retinal Ganglion cell disorders, for example, Hereditary Optic Neuropathies, Ischemic Optic Neuropathy, Nutritional and Toxic Optic Neuropathies, Optic Neuritis, Papilledema, Glaucoma, Idiopathic optic neuritis (associated with multiple sclerosis), Neuroretinitis, Devic's disease (neuromyelitis optica), Schilder's disease (demyelinating), Systemic lupus erythematosus, Sarcoid, Granulomatosis with Polyangiitis, Compressive, Grave's disease, Meningioma, Pituitary adenoma, Traumatic optic neuropathy, Ischemia, Non-arteritic anterior ischemic optic neuropathy, Arteritic anterior ischemic optic neuropathy, Posterior ischemic optic neuropathy, Toxic-Nutritional, Leber's hereditary optic neuropathy, Dominant optic neuropathy, Friedreich's ataxia, Lipidoses, Tay-Sachs, Niemann-Pick, Krabbe, Gangliosidoses, Mucopolysaccharidoses, (viii) Uveitis-associated degeneration, for example MEWDS, Acute posterior multifocal placoid pigment epitheliopathy, Behget Disease, Birdshot choroiditis, Intermediate uveitis, non-pars planitis type, Intermediate uveitis, pars planitis type, Punctate inner choroidopathy, Serpiginous choroiditis, Sympathetic Ophthalmia, Vogt-Koyanagi-Harada Syndrome, Tubulointerstitial nephritis and uveitis”.

Of particular interest according to the present disclosure is the eye disease age-related macular degeneration. In certain embodiments, the eye disease comprises “dry” age-related macular degeneration.

In another embodiment, the method further comprises administering to the subject at least one additional therapeutic agent. In some embodiments, the at least one additional therapeutic agent comprises one or more microglia checkpoint molecule inhibitors.

Another aspect of the present disclosure provides all that is described and illustrated herein.

### BRIEF DESCRIPTION OF THE DRAWINGS

The accompanying Figures and Examples are provided by way of illustration and not by way of limitation. The foregoing aspects and other features of the disclosure are explained in following description, taken in connection with the accompanying example figures (also “FIG.”) relating to one or more embodiments, in which:

FIGS. 1A-1I contain images and graphs showing subretinal microglia are a general response in mouse model of retinal degeneration and human AMD in accordance with an embodiment of the present disclosure. FIG. 1A. UMAP plot showing integrated clustering of immune cells samples from four mouse models of retinal degeneration, including light damaged (LD) model (sorted by Cx3cr1+), the acute RPE injury NaIO₃ model (CD45+), the inherited photoreceptor degeneration Rho-P23H model (CD45+) and the 2 year old mice—advanced aging model (CD45+) and naïve mice (CD45+). A total of 15,623 macrophages, including 13,489 microglia, were integrated among four models. PMN, polymorphonuclear neutrophils; mo-MFs, monocyte-derived macrophages; pv-MFs: perivascular macrophages; NK, natural killer. FIG. 1B. Uniform manifold approximation and projection (UMAP) of macrophages clusters by two datasets. Red dash circles indicate subretinal microglia (srMG). FIG. 1C. Percentage of sample distribution by clusters. The arrow indicates the enrichment of srMG cluster from degenerating retinas. FIG. 1D. Heatmap of top differentially expressed genes (DEGs) of srMG across clusters among the four mouse models. FIG. 1E-1H show upregulation of galectin-3 (Gal3) in mouse models and human AMD In situ validation of Gal3 expression in subretinal microglia. Iba1 (green), phalloidin (red) and Gal3 (magenta). Scale bar: 100 m. FIG. 1F. Percentage of Gal3+ relative to Iba1+ cells on the retinal pigment epithelium (RPE) and in the neuroretina tissues across models. FIG. 1G. Representative image of Gal3 and CD68 co-staining in the atrophic area of human AMD (Sarks' V). FIG. 1H. Correlation between Gal3+CD68+ cells (y axis) and Sarks' AMD grades (x axis) by Spearman's correlation. FIG. 1I. Percentage of Gal3+ cells relative CD68+ cells in human AMD patients (Sark III to VI). Galactin-3 is known in the art to be one of the key molecules involved in microglial activation.

FIGS. 2A-2M contain images and graphs showing loss of galectin-3 in microglia exacerbates RPE dysmorphology, photoreceptor death and vision loss in mice in accordance with one embodiment of the present disclosure. FIG. 2A-2J show loss of galectin-3 excerbatesretinal degeneration FIG. 2A, FIG. 2B. Phalloidin (red) staining in WT and Gal3 knock out (KO) RPE tissues in light damaged (LD) tissue. Quantifications of dysmorphic RPE cells are shown in FIG. 2B. WT, n=6; KO, n=7; naïve KO, n=3. FIG. 2C, FIG. 2D. TUNEL staining (green) in WT and Gal3 KO retinal cross sections in LD. DAPI, blue. Quantifications of TUNEL+ photoreceptors are shown in FIG. 2D. WT, n=5; KO, n=5; naïve KO, n=3. FIG. 2E, FIG. 2F. Representative cross sections of WT and Gal3 KO in P23H/+ mice comprising a mutation of rod opsin which causes retinal degeneration in mice. Quantifications from outer nuclear layer (ONL) thickness of the retina are shown in FIG. 2F. WT, n=7; Het, n=7; KO, n=8. FIG. 2G. Electroretinogram (ERG) data show scotopic a-waves and b-waves among WT, Gal3 Het and Gal3 KO in P23H/+ mice. WT, n=12; Het, n=6; KO, n=10. FIG. 2H, FIG. 2I. Representative images of phalloidin staining in WT and Gal3 KO RPE tissues at the age of 2 years. Quantifications of RPE cell size are shown in FIG. 2I. Dots represent individual images. N=5 mice per group. FIG. 2J. ERG data show scotopic a-waves and b-waves in 2-year-old WT and Gal3 KO mice. FIG. 2K-FIG. 2M show that Gal3-dependent RPE Protection is Microglial-specific. FIG. 2K, FIG. 2L. Representative images of dysmorphic RPE cells in microglia-specific conditional depletion of Lgals3 (Lgals3AMG) in LD compared with control. Iba1, green; phalloidin, red; Gal3, magenta. Percentage of dysmorphic RPE cells in Lgals3ΔMG are compared with genotype control (Cx3crlcre^{ER}/+Lgals3fl/fl) and tamoxifen control (Cx3cr1CreER/+ with tam) as indicated in 1. FIG. 2M. Cx3cr1CreER knock-in/knock-out mice express a Cre-ERT2 fusion protein and an enhanced yellow fluorescent protein (EYFP) from endogenous Cx3cr1 promoter/enhancer elements. Insertion of the Cre-ERT2 and EYFP knocks out endogenous CX3CR1 expression. EYFP immunofluorescence is observed in Cx3cr1-expressing microglia in the brain, mimicking endogenous gene expression patterns. Correlation matrix reveals a negative association between dysmorphic RPE cells and Gal3+ microglia.

FIGS. 3A-3I contain graphs and images showing Trem2 signaling regulates Gal3 expression in subretinal microglia in accordance with one embodiment of the present disclosure. FIG. 3A. Trem2 (red) is colocalized with Gal3 (green) in subretinal microglia (Iba1, white). Left, maximum projection (white arrow heads indicate the colocalization), right single plane of confocal image (blue arrows indicate the direction of apical aspect of the RPE) FIG. 3B depicts a rendering of Trem2 and Gal3 show the colocalization is facing on the apical aspect of the RPE but not neuroretina. FIG. 3C. Representative images of Trem2 conditional depletion (Trem2ΔMG) in LD. Iba1, green; Trem2, red. FIG. 3D. Representative images of phalloidin staining in control and Trem2ΔMG in LD. FIG. 3E. Representative images of Gal3 expression in Trem2ΔMG compared with control. FIGS. 3F and 3G show Trem2 inhibition or depletion impairs subretinal protection by microglia. (Trem2 antagonist mab 178 anti-Trem2 (molgoro et al. Cell 2020); Trem2 conditional knockout: Cx3cr1^{YFP-CreER/+}; Trem2^{fl/fl.} FIG. 3F. Representative fundus images of mice treated with vehicle control or mAb178 inhibitory anti-Trem2. Individual images from 4 mice per group from 2 independent experiments are shown. FIG. 3G. Quantifications of dysmorphic RPE cells between mAbl78 inhibitory anti-Trem2 or vehicle control in LD. Vehicle, n=8; mAbl78, n=9. FIG. 3H and FIG. 3I. Quantifications of Gal3+ microglia on the RPE between mAb178 inhibitory anti-Trem2 or vehicle control in LD. Vehicle, n=8; mAb178, n=9.

FIGS. 4A-4H contain graphs and images showing that bolstering Gal3-dependent Trem2 signaling prevents retinal degeneration in accordance with one embodiment of the present disclosure. FIG. 4A-C shows TREM2 agonist bolsters protection in retinal degradation. FIG. 4A. Representative fundus images of mice treated with isotype control or 4D9 anti-Trem2 in LD. Individual images from 4 mice per group from 3 independent experiments are shown. FIG. 4B, FIG. 4C. Representative Optical coherence tomography (OCT) images of mice treated with isotype control or 4D9 anti-Trem2 in LD. Quantifications of outer nuclear layer (ONL) thickness by OCT are shown in c. N=26 eyes from 13 mice per group. FIGS. 4D-F show added protection is microglial mediated and gal3 dependent. FIG. 4D. Representative fundus images of Lgals3^{ΔMG} mice treated with isotype control or 4D9 anti-Trem2 in LD. FIG. 4E, FIG. 4F. Representative OCT images of Lgals3^{ΔMG} mice treated with isotype control or 4D9 anti-Trem2 in LD. Quantifications of ONL thickness by OCT are shown in f. Control isotype or 4D9, N=13; Lgals3ΔMG isotype, N=12; Lgals3^{ΔMG} 4D9, N=10. FIG. 4G, FIG. 4H. Representative RPE phalloidin staining in control and Lgals3ΔMG mice treated with isotype control or 4D9 anti-Trem2 in LD. Quantifications of dysmorphic RPE cells are shown in h. Control isotype, N=15; control 4D9, N=13; Lgals3^{ΔMG} isotype, N=11; Lgals3^{ΔMG} 4D9, N=13.

FIGS. 5A-5C supplement FIGS. 1A-1I and contain single cell data from FIGS. 1A-1I. FIG. 5A and FIG. 5B depict UMAP plots showing retinal CD45+ cells collected from NaIO3 mediated RPE injury model, P23H model, and aging model and naïve mice as indicated. FIG. 5C depicts violin plots showing marker expression for each cluster.

FIGS. 6A-6E supplement FIGS. 3A-3I, providing further Gal3 data indicating loss of Gal3 exacerbates disease-related pathology but not subretinal abundance of Iba1+ cells. FIG. 6A depicts Iba1 (green) and phalloidin (red) staining in RPE flat mounts from LD-subjected mice as indicated. FIG. 6B depicts quantifications of subretinal Iba1 frequencies as shown in FIG. 6A. FIG. 6C depicts Iba1 (green) and phalloidin (red) staining in RPE flat mounts from P23H mice as indicated. FIG. 6D depicts quantifications of subretinal Iba1 frequencies as shown in FIG. 6C. FIG. 6E depicts Representative ERG responses at different flash intensities as indicated. Scale bars: 100 m.

FIG. 7A, FIG. 7B and FIG. 7C supplement FIGS. 3A-3I.

FIG. 8 supplements FIGS. 4A-4H.

FIG. 9A and FIG. 9B relate to the generation of antibodies which bind to human TREM2 close to the ADAM10/17 cleavage site. Antibodies generated from FIG. 9A provides an excerpt of the amino acid sequence of human TREM2, and further depicts the amino acid sequence of each of 2 peptides (one peptide consisting of residues 145-155 and a second peptide consisting of residues 153-162) used to immunize rats. Antibodies generated by immunization with the former peptide are termed “T2GE” antibodies and antibodies generated by immunization with the latter peptide are termed “TREMX” antibodies. FIG. 9B depicts the epitope of antibody 4D9 (Schlepov et al (2020)) with respect to mouse TREM2.

FIG. 10A and FIG. 10B show that T2GE antibodies block shedding of cell surface TREM2 Immunoblot analysis of immunoprecipitants using 4D9 antibody against mouse TREM2 (FIG. 10A (Schlepckow et al. (2000) EMBO Mol. Med.)) and “TREMX” and “T2GE” antibodies against human TREM2 (FIG. 10B) showing blocking of shedding of cell surface TREM2. Immunoblotting of soluble TREM2 (sTREM2) immunoprecipitated from conditioned media and mature/immature TREM2 from membrane fractions of HEK cells stably overexpressing wt TREM2 upon 24 hrs antibody treatment using the indicated clones.

FIG. 11 shows that T2GE antibodies directed against human TREM2 have an IC50 of shedding inhibition in the low nM range.

FIG. 12A and FIG. 12B display date used to determine the IC50 of soluble TREM2 mediated by the indicated antibodies directed against human TREM2.

FIG. 13A shows that T2GE antibodies directed against human TREM2 activate pSYK. FIG. 13B shows that antibody 4D9 directed against mouse TREM2 activate pSYK.

FIG. 14A shows that T2GE antibodies directed against human TREM2 boost liposome-mediated pSYK signalling. FIG. 14B shows that antibody 4D9 directed against mouse TREM2 boost liposome-mediated pSYK signalling.

### DESCRIPTION

For the purposes of promoting an understanding of the principles of the present disclosure, reference will now be made to preferred embodiments and specific language will be used to describe the same.

It will nevertheless be understood that no limitation of the scope of the disclosure is thereby intended, such alteration and further modifications of the disclosure as illustrated herein, being contemplated as would normally occur to one skilled in the art to which the disclosure relates.

### Definitions

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result. In some embodiments, the term “about” as used herein means that values of 10% or less above or below the indicated values are also included.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is of particular interest according to the present disclosure is an eye disease comprising age-related macular degeneration. In certain embodiments, the eye disease comprises “dry” age-related macular degeneration.

AMD refers to age-related degeneration of the macula, which is the leading cause of irreversible visual dysfunction in individuals over the age of 60. Two types of AMD exist, non-exudative (dry) and exudative (wet) AMD. The dry, or nonexudative, form involves atrophic and hypertrophic changes in the retinal pigment epithelium (RPE) underlying the central retina (macula) as well as deposits (drusen) on the RPE. Patients with nonexudative AMD can progress to the wet, or exudative, form of AMD, in which abnormal blood vessels called choroidal neovascular membranes (CNVMs) develop under the retina, leak fluid and blood, and ultimately cause a blinding disciform scar in and under the retina. Nonexudative AMD, which is usually a precursor of exudative AMD, is more common. The presentation of nonexudative AMD varies; hard drusen, soft drusen, RPE geographic atrophy, and pigment clumping can be present. Complement components are deposited on the RPE early in AMD and are major constituents of drusen.

The retina is a stratiform sensory tissue that consists of various cell types, including retinal pigment epithelium (RPE) cells, photoreceptors, intermediate neurons, retinal ganglion cells (RGCs) and glial cells (Malhotra et al., 2011; Madeira et al., 2015). Microglia, is one of three distinct glial cell types present within the retina and are distributed widely in the whole retina displaying phagocytosis and regulation of tissue inflammation. Two phenotypes of microglia have been identified: M1 microglia and M2 microglia. The former phenotype is generally considered pro-inflammatory, while the latter phenotype is anti-inflammatory (Tang and Le, 2016; Jiang et al., 2020); see Jin et al. Frontiers in Cell and Developmental Biology 13 Dec. 2021; Sec. Stem Cell Research (https://doi.org/10.3389/fcell.2021.741368).

TREM2 cleavage, as used herein, refers to cleavage of human TREM2 on the surface of retinal microglial cells and mediated by ADAM610 and/or ADAM17, and/or cleavage between residues 157 and 158 of human TREM2.

Inhibition of cleavage of TREM2, as used herein, correlates with increased human TREM2 on the surface of retinal microglial cells.

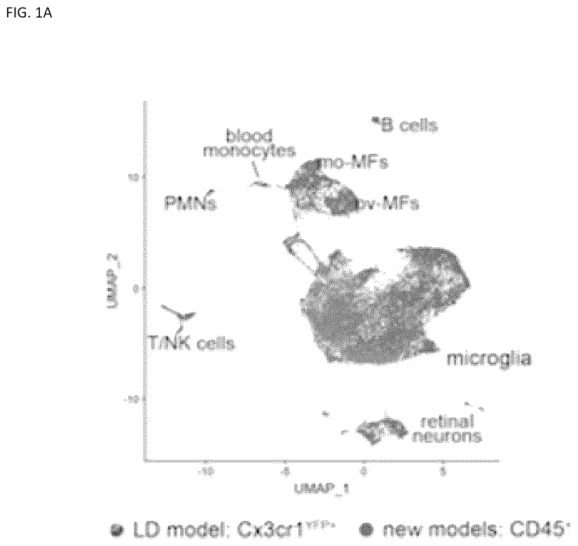

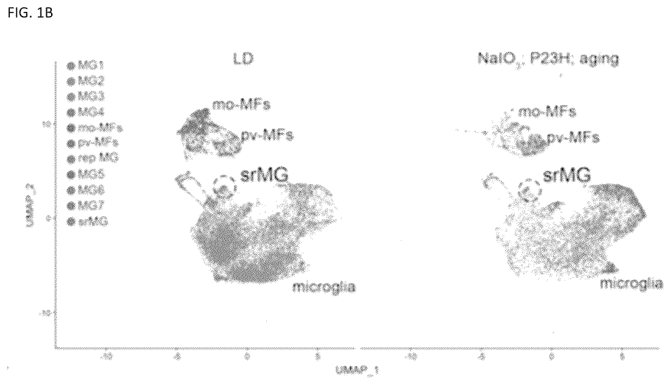

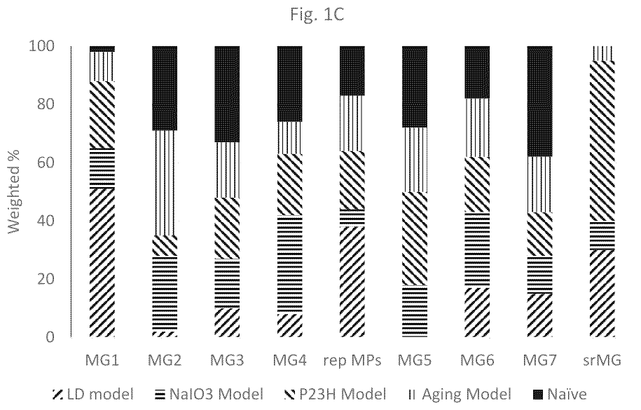

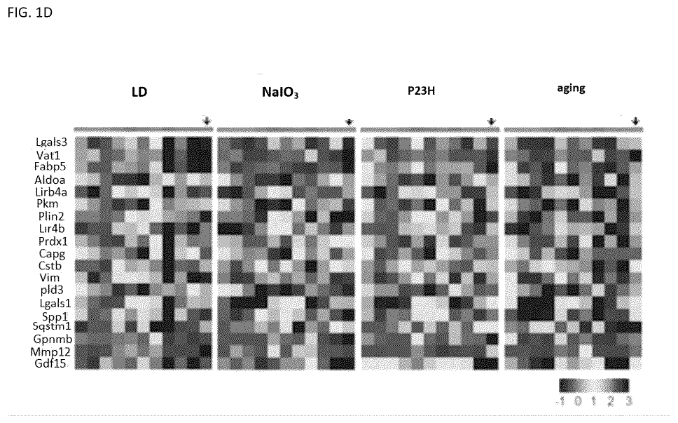

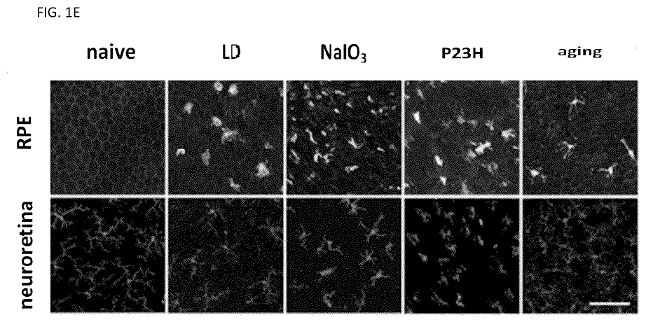

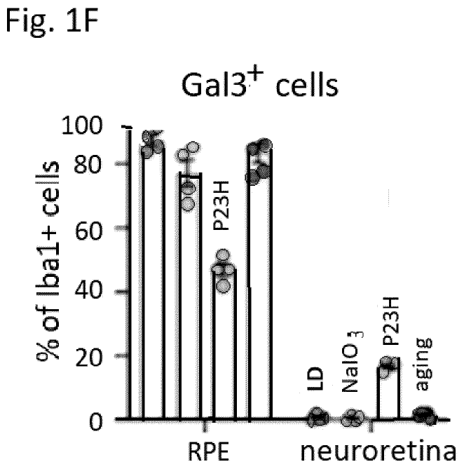

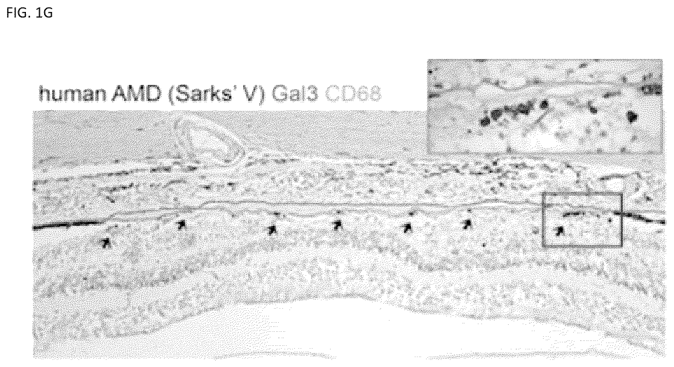

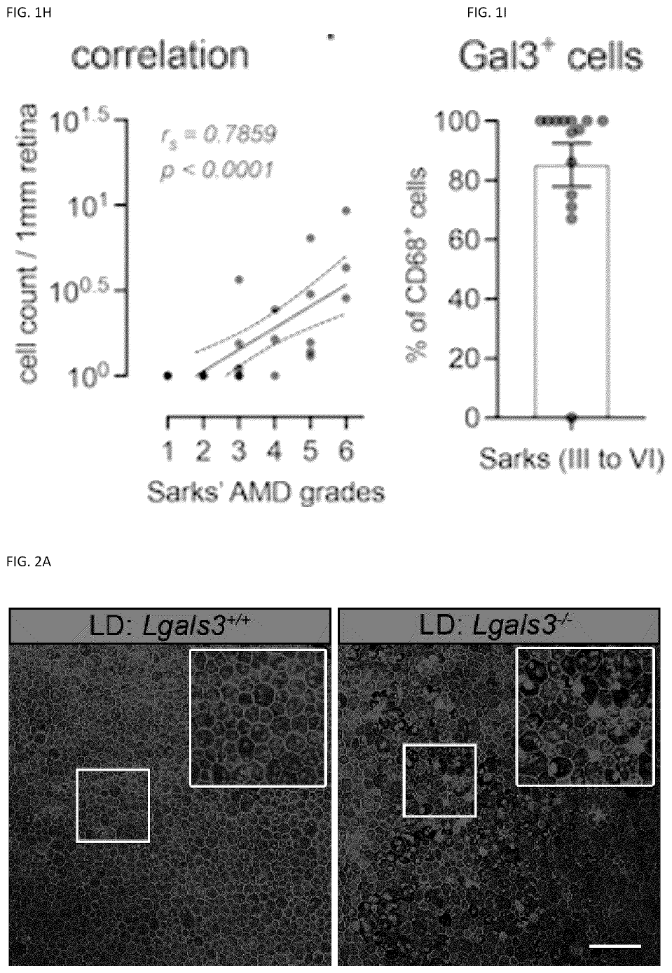

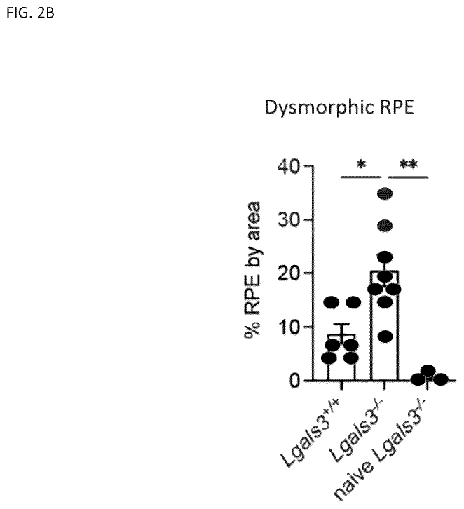

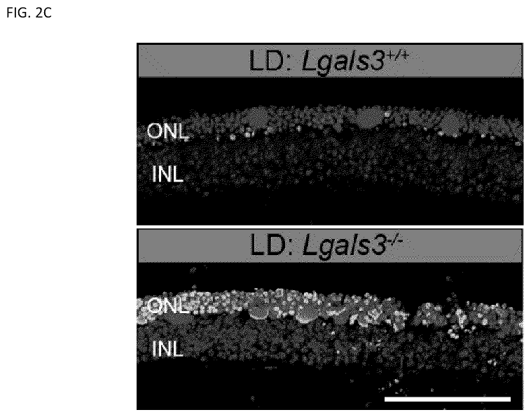

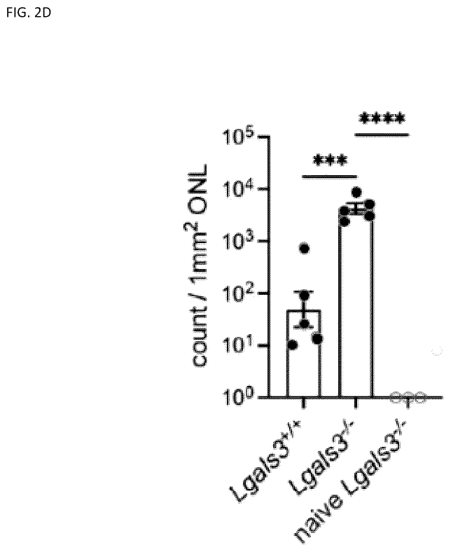

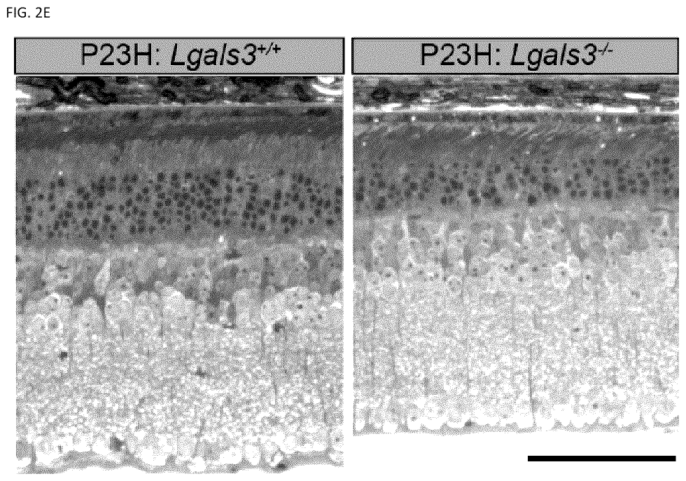

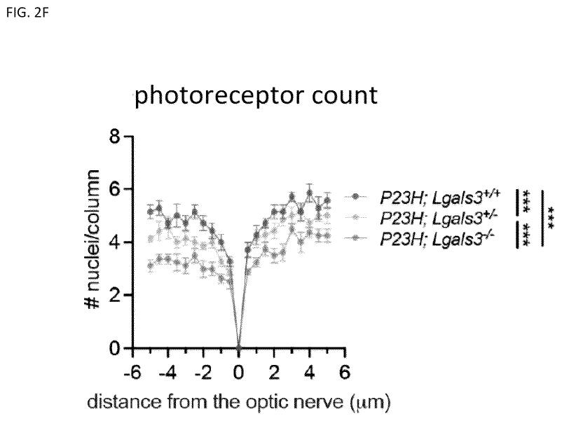

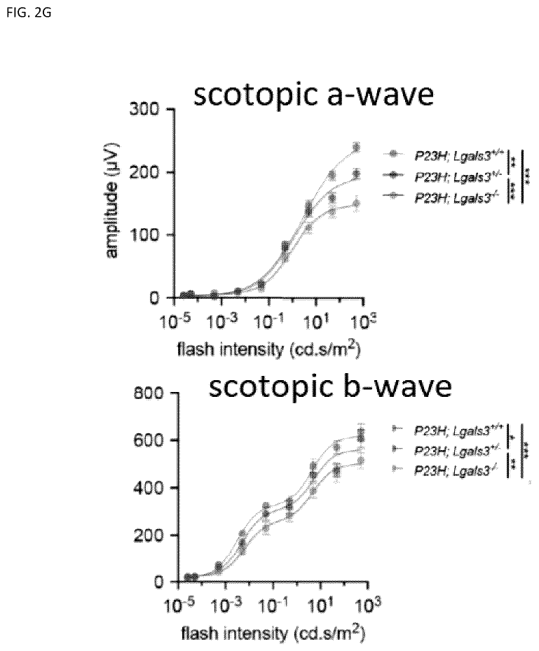

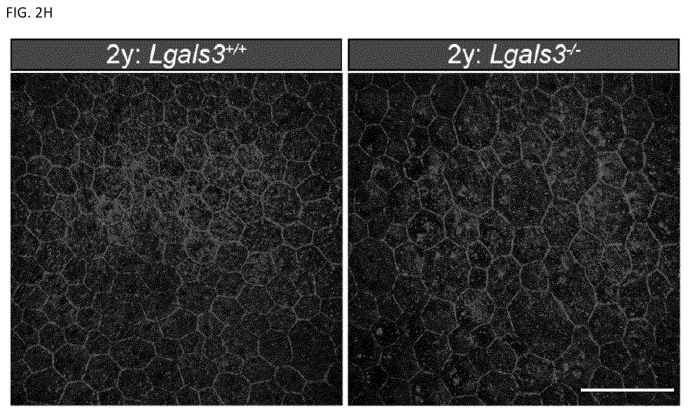

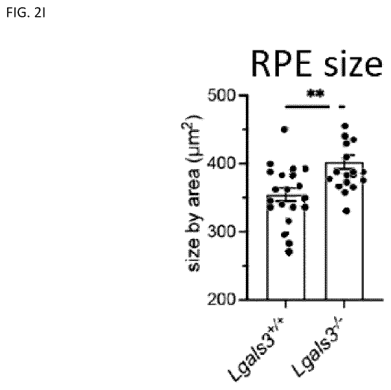

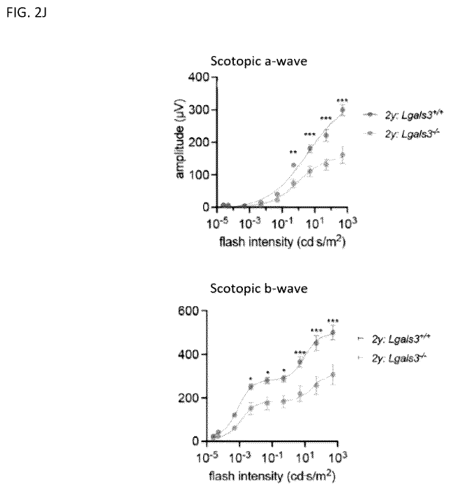

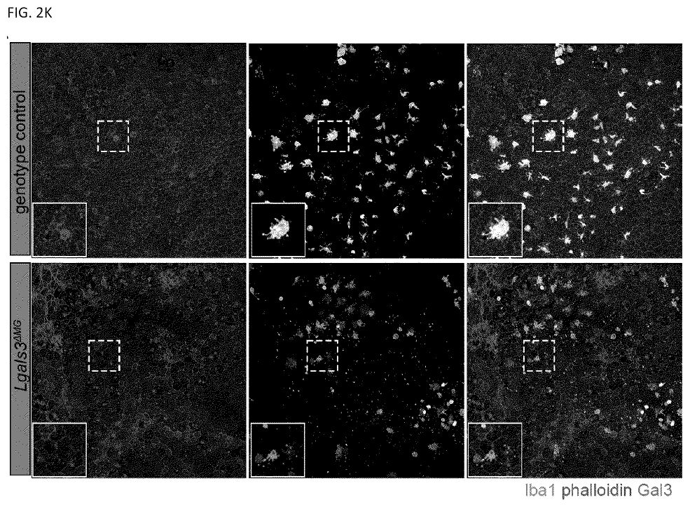

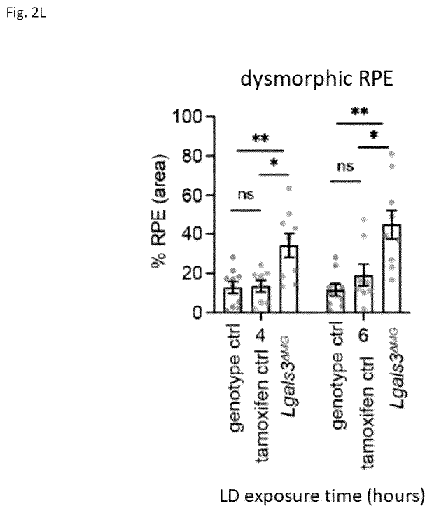

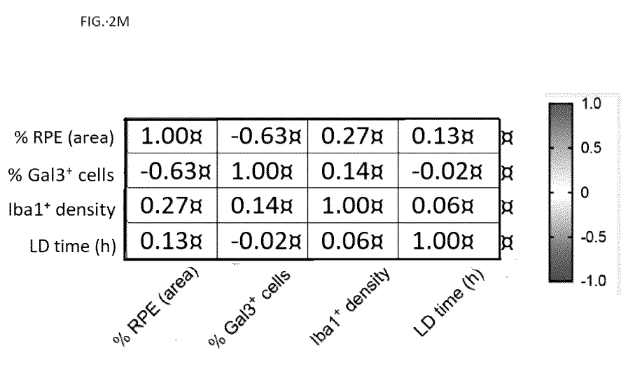

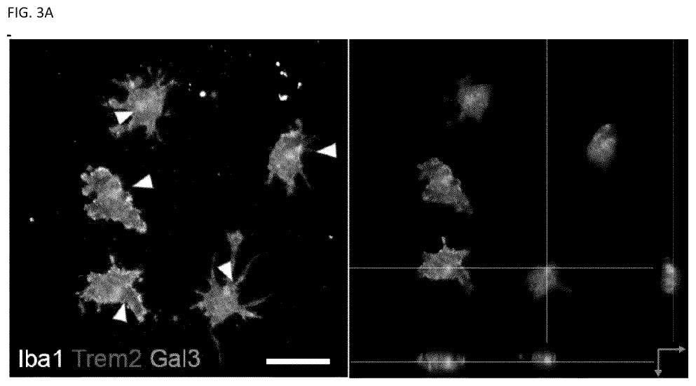

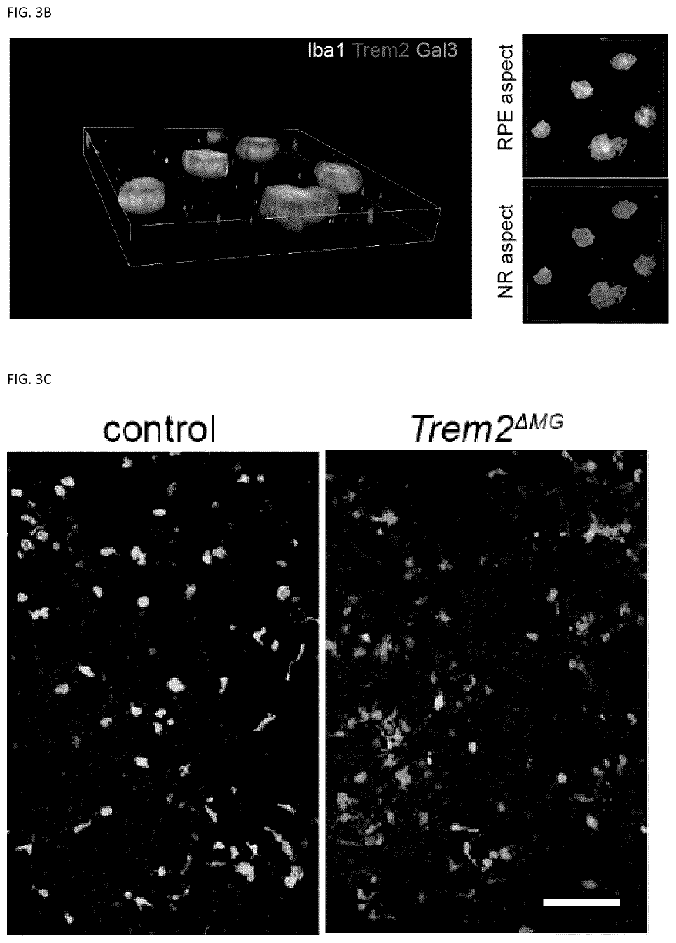

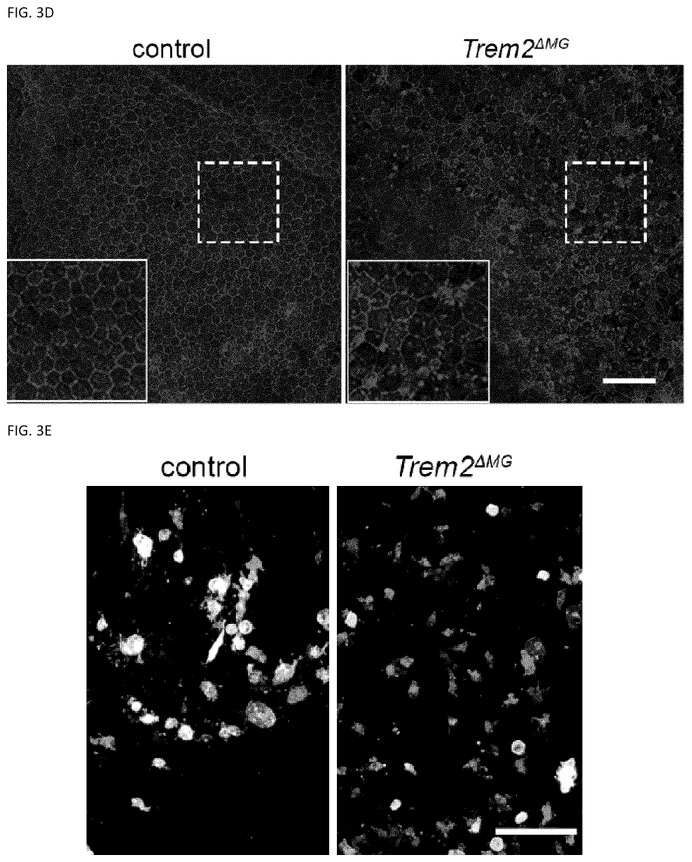

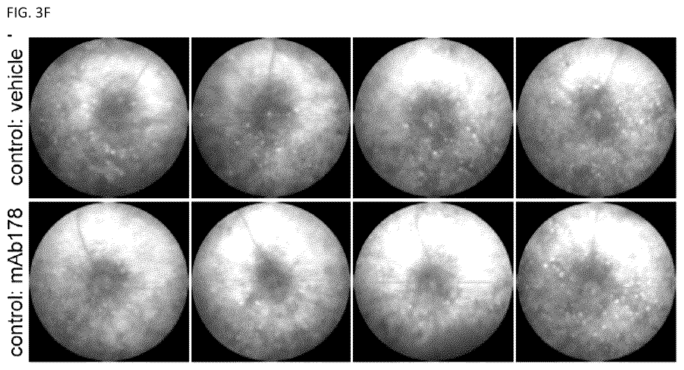

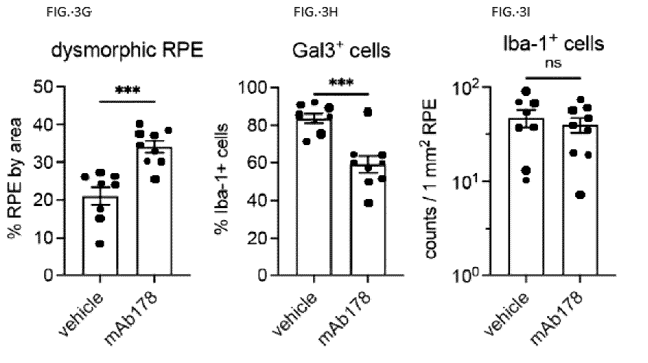

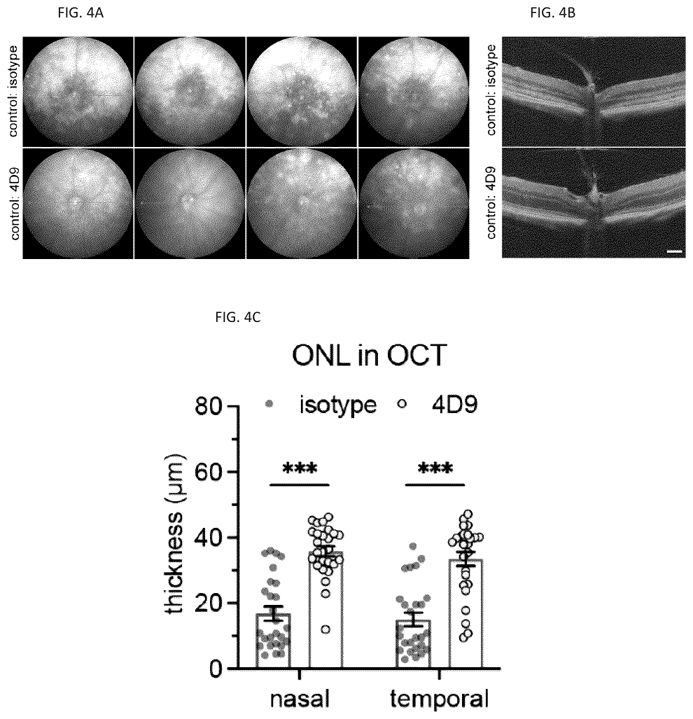

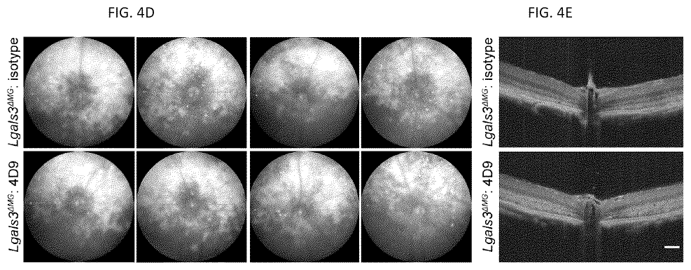

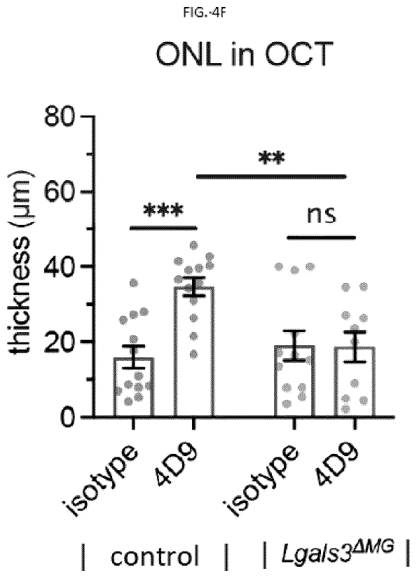

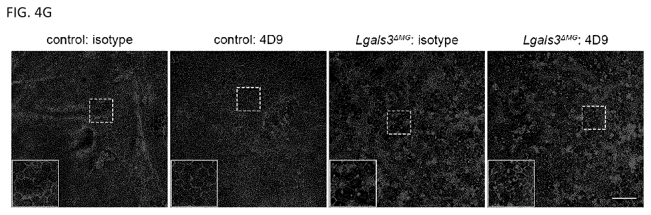

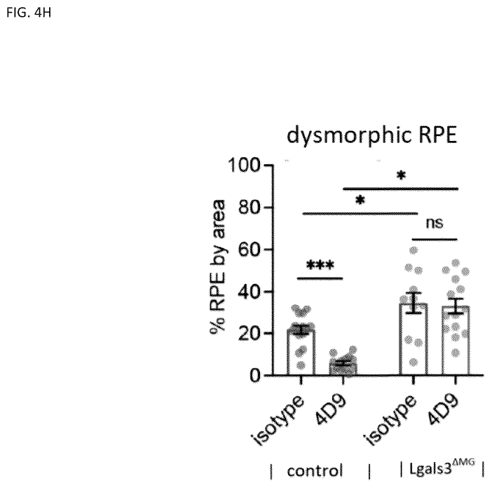

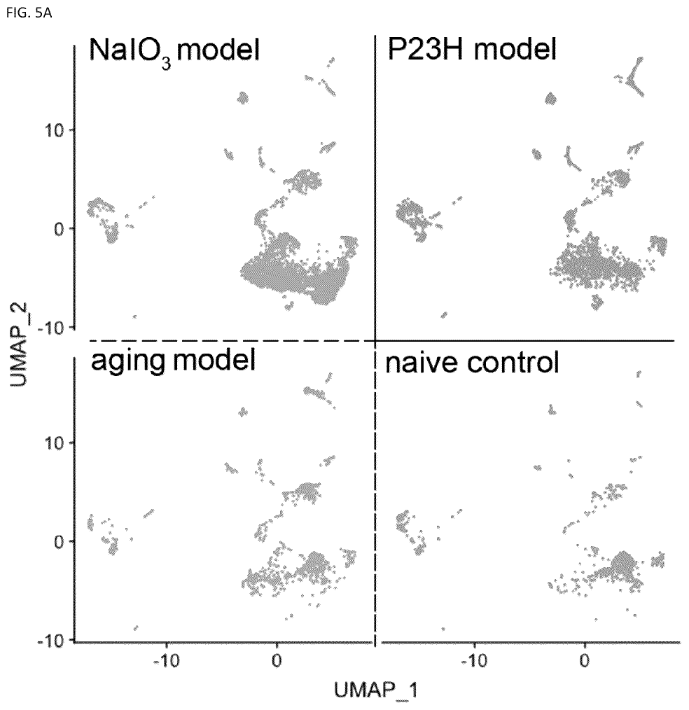

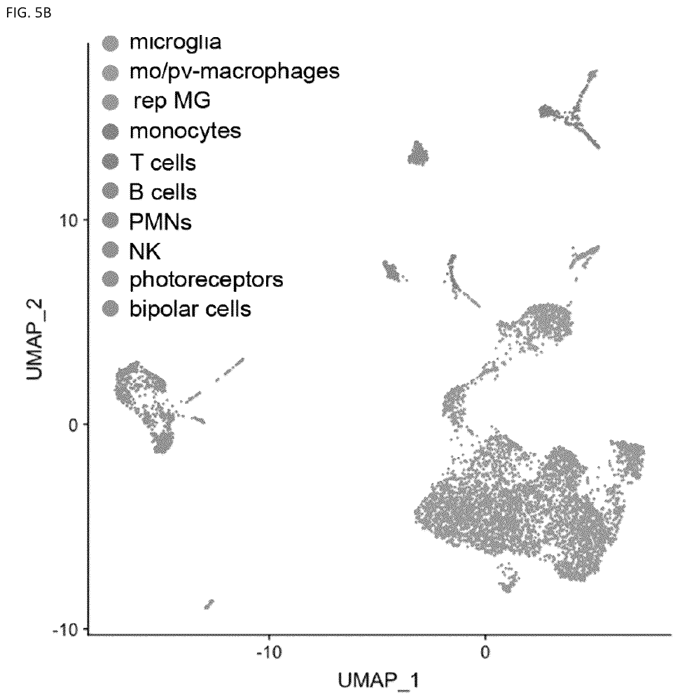

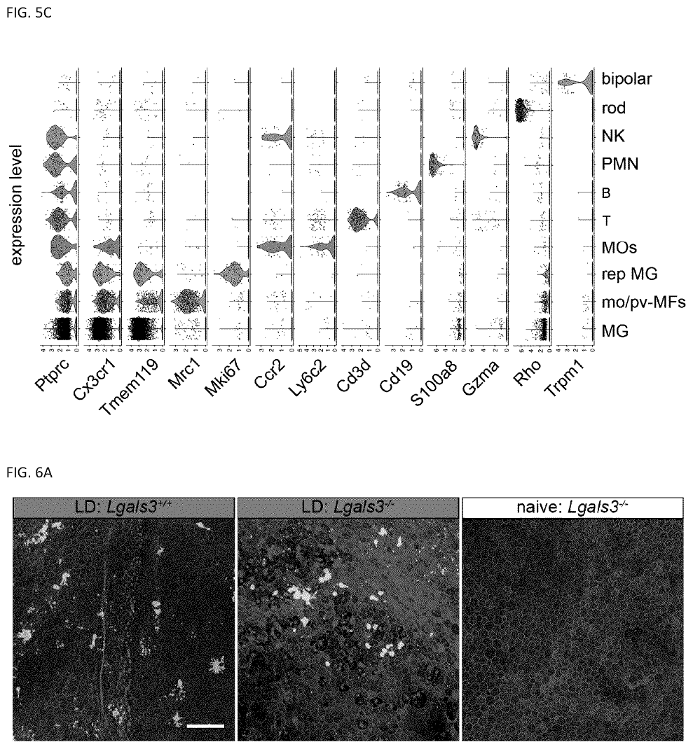

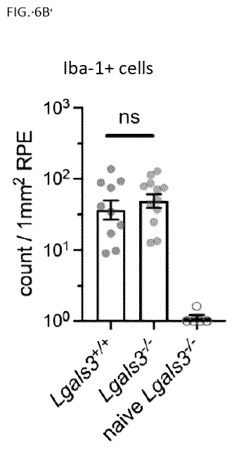

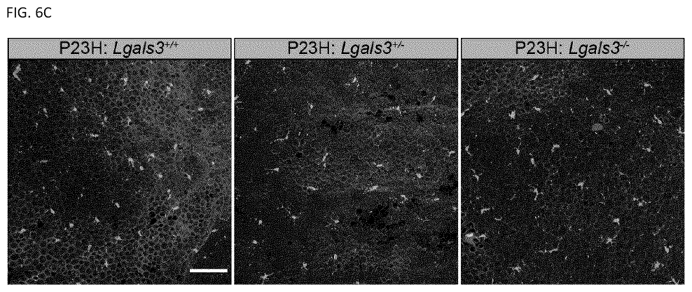

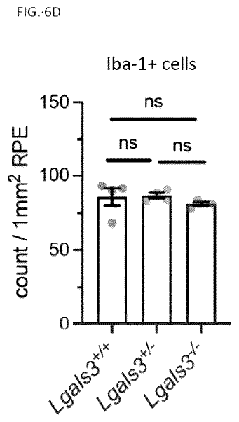

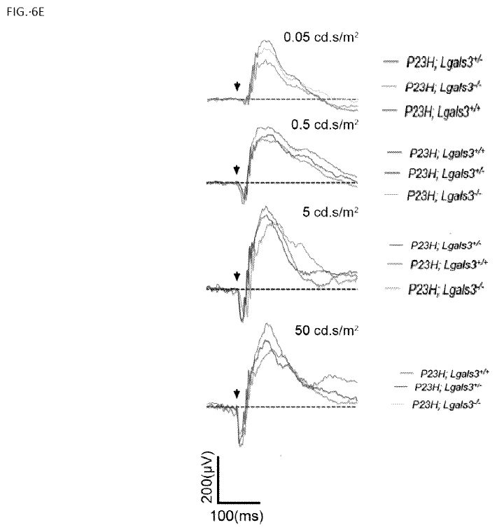

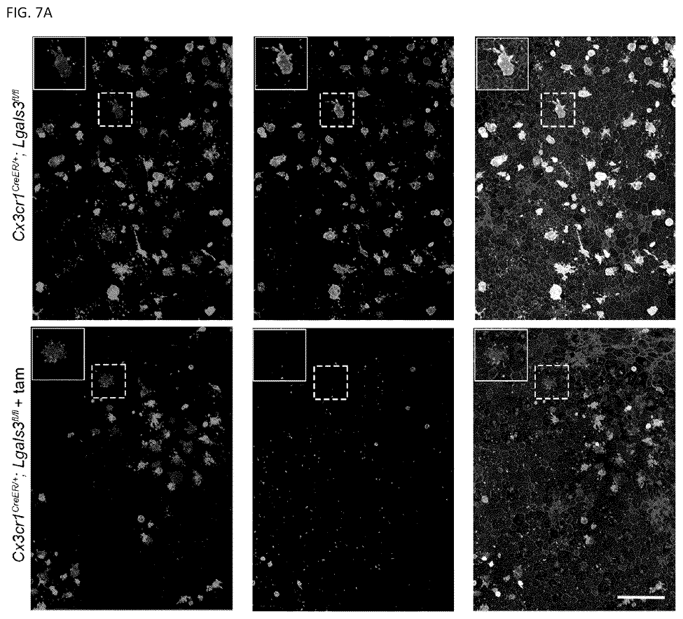

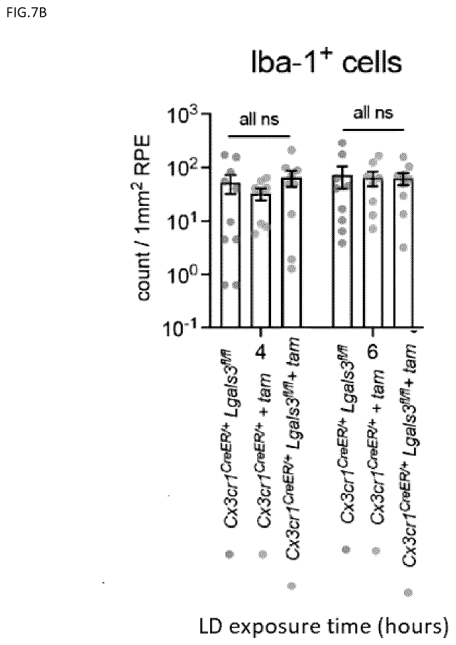

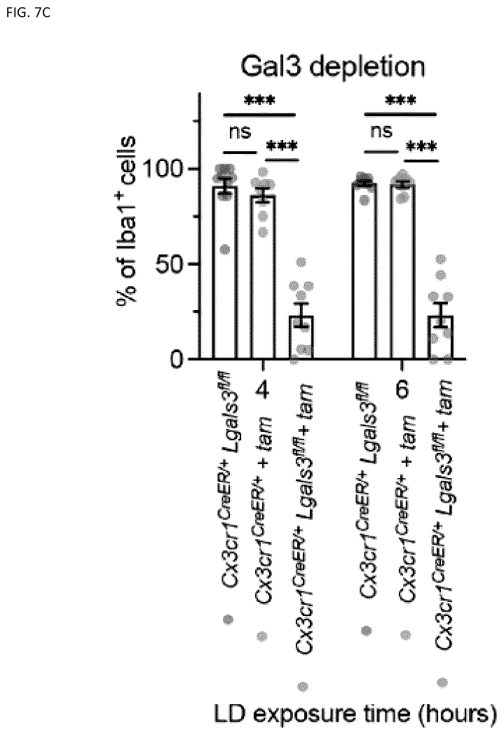

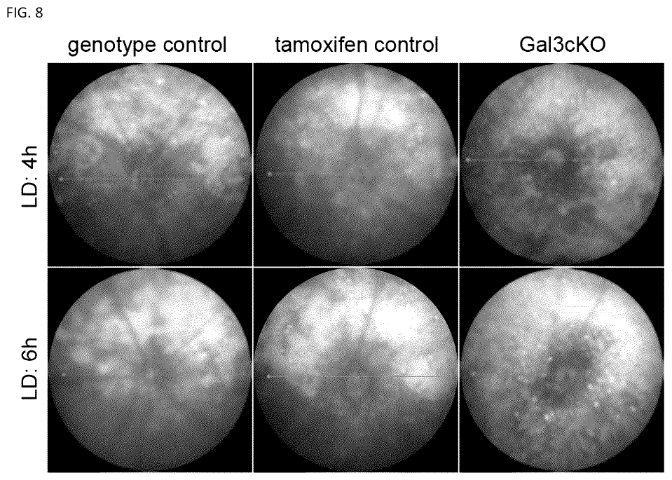

Full-length TREM2, in association with DAP12, forms a heteromeric complex required to activate phospho-SYK signaling (Colonna, 2003). Signaling appears to be terminated by α-secretase-mediated shedding of the TREM2 ectodomain (FIG. 1A) (Wunderlich et al, 2013; Kleinberger et al, 2014. Loss of TREM2-mediated signaling locks microglia in a homeostatic state and inhibits their transition to disease-associated microglia (DAM) (Krasemann et al, 2017; Mazaheri et al, 2017), which are phenotypically characterized by enhanced migration, chemotaxis, and phagocytosis (Keren-Shaul et al, 2017; Mazaheri et al, 2017), Schlepckow et al (2020) EMBO 12(4):e 11227.

A binding molecule may be an antibody (such as a nanobody) or a small molecule. A “small molecule” may be of any kind including peptides, foldamers, proteomimetics and compounds derived from organic synthesis with a low molecular weight (<900 daltons). Small molecules may help to regulate a biological process, and have generally a size on the order of 10-10 m. Many drugs are small molecules.

Herein, the terms “peptide”, “oligopeptide”, “polypeptide” and “protein” are used interchangeably and relate to a molecule that encompasses at least one amino acid chain, wherein the amino acid residues are linked by peptide (amide) bonds. The terms “peptide”, “oligopeptide”, “polypeptide” and “protein” also encompass molecules comprising amino acids other than the 20 gene-encoded amino acids, such as selenocysteine. Herein the terms “peptide”, “oligopeptide”, “polypeptide” and “protein” also include molecules with modifications, such as glycosylation, acetylation, phosphorylation, ubiquitination, sumolyation and the like. Such modifications are well described in the art.

Herein, the term “proteomimetic” refers to any compound that mimics the structure and function of a region of protein (or polypeptide, or oligopeptide, or peptide) surface.

The binding molecule of the present invention binds the stalk region of human TREM2 on the surface of retinal microglial cells.

However, as described above, the binding molecule of the present invention may also be an antibody. Preferably, the antibody is a monoclonal antibody. The antibody may also be an antibody fragment, such as a nanobody, a Fab fragment, a Fab′ fragment, a Fab′-SH fragment, a F(ab′)2 fragment, a Fd fragment, a Fv fragment, a scFv fragment, or an isolated complementarity determining region (CDR). The antibody or antibody fragment may be a humanized antibody/antibody fragment, a fully human antibody/antibody fragment, a mouse antibody/antibody fragment, a rat antibody/antibody fragment, a rabbit antibody/antibody fragment, a hamster antibody/antibody fragment, a goat antibody/antibody fragment, a guinea pig antibody/antibody fragment, a ferret antibody/antibody fragment, a cat antibody/antibody fragment, a dog antibody/antibody fragment, a chicken antibody/antibody fragment, a sheep antibody/antibody fragment, a bovine antibody/antibody fragment, a horse antibody/antibody fragment, a camel antibody/antibody fragment, or a monkey antibody/antibody fragment such as a primate antibody/antibody fragment. It is prioritized that the antibody is a humanized antibody/antibody fragment, a fully human antibody/antibody fragment, a mouse antibody/antibody fragment, a rat antibody/antibody fragment, a rabbit antibody/antibody fragment, a hamster antibody/antibody fragment, a goat antibody/antibody fragment, a guinea pig antibody/antibody fragment, a ferret antibody/antibody fragment, a chicken antibody/antibody fragment, a sheep antibody/antibody fragment, or a monkey antibody/antibody fragment such as a primate antibody/antibody fragment. It is even more prioritized that the antibody is a humanized antibody/antibody fragment, a fully human antibody/antibody fragment, a mouse antibody/antibody fragment, or a rat antibody/antibody fragment. Accordingly, the herein provided binding molecule may be a humanized antibody fragment, such as a humanized nanobody. The herein provided binding molecule may further be a chimeric antibody and/or a bispecific antibody.

The term “fully-human antibody” as used herein refers to an antibody that comprises human immunoglobulin protein sequences only.

Herein the term “antibody” includes a peptide or polypeptide derived from, modelled after or substantially encoded by an immunoglobulin gene or immunoglobulin genes, or fragments thereof, capable of specifically binding an antigen or epitope, see, e.g. [WE, P., Fundamental Immunology, Third Edition. Third Edition ed, ed. W. E. Paul. 1993: Raven Press, N.Y.; Wilson, K. M., et al., Simplified conjugation chemistry for coupling peptides to F(ab) fragments: autologous red cell agglutination assay for HIV-1 antibodies. J Immunol Methods, 1994. 175(2): p. 267-73; and Yarmush, M. L., X. M. Lu, and D. M. Yarmush, Coupling of antibody-binding fragments to solid-phase supports: site-directed binding of F(ab′)2 fragments. J Biochem Biophys Methods, 1992. 25(4): p. 285-97]. The term “antibody” includes antigen-binding portions, i.e., “antigen binding sites,” (e.g., fragments, subsequences, or complementarity determining regions (CDRs)) that retain capacity to bind an antigen (such as TREM2), comprising or alternatively consisting of, for example, (i) a Fab fragment, i.e. a monovalent fragment consisting of the VL, VH, CL and CH1 domains; (ii) a F(ab′)₂ fragment, i.e. a bivalent fragment comprising two Fab fragments linked by a disulfide bridge at the hinge region; (iii) a Fab′ fragment, i.e. one of the two fragments that are formed if a F(ab′)₂ fragment is split by mild reduction; (iv) a Fab′-SH fragment, i.e. a Fab′ fragment containing a free sulfhydryl group; (v) a Fd fragment consisting of the VH and CH1 domains; (vi) a Fv fragment consisting of the VL and VH domains of a single arm of an antibody; (vii) a scFv fragment, i.e. a single-chain variable fragment, wherein the variable regions of the heavy and light chains are fused together; or (viii) an isolated complementarity determining region (CDR). The herein provided antibody fragment may also be (ix) a dAb fragment, which consists of a VH domain (see, e.g. Ward et al. [Ward, E. S., et al., Binding activities of a repertoire of single immunoglobulin variable domains secreted from Escherichia coli. Nature, 1989. 341(6242): p. 544-6]).

An antigen binding region (ABR) of an antibody is predicted by: https://ofranservices.biu.ac.il/site/services/paratome/index.html.

The antibody fragment may also be a single-domain antibody, sdAb. Single-domain antibodies are also called nanobody; see, e.g., Gibbs, 2005, “Nanobodies”, Scientific American Magazine. A sdAb or nanobody is an antibody fragment consisting of a single monomeric variable antibody domain. With a molecular weight of only 12-15 kDa, single-domain antibodies are much smaller than common antibodies (150-160 kDa) that are composed of two heavy protein chains and two light chains, and even smaller than Fab fragments (^{˜}50 kDa, one light chain and half a heavy chain) and single-chain variable fragments (^{˜}25 kDa, two variable domains, one from a light and one from a heavy chain); see, e.g. [Harmsen, M. M. and H. J. De Haard, Properties, production, and applications of camelid single-domain antibody fragments. Appl Microbiol Biotechnol, 2007. 77(1): p. 13-22]. The term “antibody” also relates to bispecific (i.e. bifunctional) antibodies. The term “bispecific antibody” as used herein refers to an artificial hybrid antibody having two different heavy/light chain pairs and two different binding sites.

A fully human antibody may contain murine carbohydrate chains if produced in a mouse, in a mouse cell or in a hybridoma derived from a mouse cell. Alternatively, a “fully-human antibody” may contain rat carbohydrate chains if produced in a rat, in a rat cell, in a hybridoma derived from a rat cell. Similarly, “(fully-)mouse antibody” or “(fully-) murine antibody” refers to an antibody that comprises mouse (murine) immunoglobulin protein sequences only. The term “(fully-) rat antibody” refers to an antibody that comprises rat immunoglobulin sequences only. In line with this the terms “(fully-)rabbit antibody”, “(fully-)hamster antibody”, “(fully-)goat antibody”, “(fully-)guinea pig antibody”, “(fully) ferret antibody”, “(fully-)cat antibody”, “(fully-)dog antibody”, “(fully-)chicken antibody”, “(fully-)sheep antibody”, “(fully-) bovine antibody”, “(fully-)horse antibody”, “(fully-)camel antibody” and “(fully-)monkey antibody” refer to an antibody that comprises rabbit, hamster, goat, guinea pig, ferret, cat, dog, chicken, sheep, bovine, horse, camel, or monkey, respectively, immunoglobulin sequences only.

The herein provided antibody may also be a chimeric antibody. The term “chimeric antibody” refers to an antibody that comprises a variable region of a human or non-human species fused or chimerized with an antibody region (e.g., constant region) from another, human or non-human species (e.g., mouse, horse, rabbit, dog, cow, chicken).

Herein, the term “antibody” also relates to recombinant human antibodies, heterologous antibodies and heterohybrid antibodies. The term “recombinant human antibody” includes all human sequence antibodies that are prepared, expressed, created or isolated by recombinant means, such as antibodies isolated from an animal (e.g., a mouse) that is transgenic for human immunoglobulin genes; antibodies expressed using a recombinant expression vector transfected into a host cell, antibodies isolated from a recombinant, combinatorial human antibody library; or antibodies prepared, expressed, created or isolated by any other means that involves splicing of human immunoglobulin gene sequences to other DNA sequences. Such recombinant human antibodies have variable and constant regions (if present) derived from human germline immunoglobulin sequences. Such antibodies can, however, be subjected to in vitro mutagenesis (or, when an animal transgenic for human Ig sequences is used, in vivo somatic mutagenesis); and thus, the amino acid sequences of the VH and VL regions of the recombinant antibodies are sequences that, while derived from and related to human germline VH and VL sequences, may not naturally exist within the human antibody germline repertoire in vivo.

The binding molecule of the present invention may be a naturally occurring molecule, e.g. a naturally occurring antibody. However, the binding molecule of the present invention may also be a non-naturally occurring molecule. For example, the binding molecule of the invention may be an antibody having an amino acid sequence that is not identical to naturally occurring antibodies or may be an antibody comprising at least one non-naturally occurring amino acid residue such as synthetic amino acids providing similar side chain functionality. For example, aromatic amino acids may be replaced with D- or L-naphthylalanine, D- or L-phenylglycine, D- or L-2-thienylalanine, D- or L-1-, 2-, 3-, or 4-pyrenylalanine, D- or L-3-thienylalanine, D- or L-(2-pyridinyl)-alanine, D- or L-(3-pyridinyl)-alanine, D- or L-(2-pyrazinyl)-alanine, D- or L-(4-isopropyl)phenylglycine, D-(trifluoromethyl)-phenylglycine, D-(trifluoromethyl)-phenylalanine, D-p-fluorophenylalanine, D- or L-pbiphenylalanine D- or L-p-methoxybiphenylalanine, D- or L-2-indole(alkyl)alanines, and D- or L-alkylalanines wherein the alkyl group is selected from the group consisting of substituted or unsubstituted methyl, ethyl, propyl, hexyl, butyl, pentyl, isopropyl, iso-butyl, and iso-pentyl. Non-carboxylate amino acids can be made to possess a negative charge, as provided by phosphono- or sulfated amino acids, which are to be considered as non-limiting examples. Further non-natural amino acids are alkylated amino acids, made by combining an alkyl group with any natural amino acid. Basic natural amino acids such as lysine and arginine may be substituted with alkyl groups at the amine (NH2) functionality. Yet other substitutions on non-natural amino acids include nitrile derivatives (e.g., containing a ON-moiety in place of the CONH2 functionality) of asparagine or glutamine, and sulfoxide derivative of methionine.

Preferably, the herein provided binding molecule specifically binds to the ectodomain of TREM2. The phrase “specifically bind(s)” or “bind(s) specifically” when referring to a binding molecule refers to a binding molecule which has intermediate or high binding affinity, exclusively or predominately, to a target molecule, such as the ectodomain of TREM2. The phrase “specifically binds to” refers to a binding reaction which is determinative of the presence of a target protein (such as the ectodomain of TREM2) in the presence of a heterogeneous population of proteins and other biologics. Thus, under designated assay conditions, the specified binding molecules bind preferentially to a particular target protein (e.g. the ectodomain of TREM2) and do not bind in a significant amount to other components present in a test sample. Specific binding to a target protein under such conditions may require a binding molecule that is selected for its specificity for a particular target protein. A variety of assay formats may be used to select binding molecules that are specifically reactive with a particular target protein. For example, solid-phase ELISA immunoassays, immunoprecipitation, Biacore and Western blot may be used to identify binding molecules that specifically bind to the ectodomain of TREM2. Typically, a specific or selective reaction will be at least twice background signal or noise and more typically more than 10 times background. Given that preferably the binding molecule is an antibody, the phrase “specifically binds to” refers to a binding reaction that is determinative of the presence of the antigen (such as the ectodomain of TREM2) in a heterogeneous population of proteins and other biologics. Typically, an antibody that specifically binds to its antigen binds said antigen with an association constant (Ka) of at least about 1×106 M−1 or 107 M−1, or about 108 M−1 to 109 M−1, or about 1010 M−1 to 1011 M−1 or higher; and/or binds to the predetermined antigen (e.g. the ectodomain of TREM2) with an affinity that is at least two-fold greater than its affinity for binding to a non-specific antigen (e.g., BSA, casein) other than the predetermined antigen or a closely-related antigen.

### DETAILED DESCRIPTION OF THE INVENTION

Enhancing Protective Microglial Activities with a Dual Function TREM2 Antibody to the Stalk Region

The present disclosure is based, in part, on the discovery by the inventors that an established genetic risk factor in one disease revealed a potentially fundamental and ‘drugable’ pathobiology in a separate disease. The former is represented by Alzheimer's disease, whereas the latter is AMD, a form of retinal degeneration associated with aging. The inventors further show that TREM2—a genetic risk factor in Alzheimer's disease—is pertinent in the pathobiology of AMD and that targeting its pathway restricts disease progression. The basis of these findings came about by studying microglia—the resident macrophages of retina and throughout the central nervous system.

### Examination of TREM2 in Alzheimer's Mouse Model.

Functional studies in Alzheimer's mouse models demonstrated that microglia clear and contain toxic neuritic debris and form a neuroprotective barrier around amyloid plaques. TREM2 contributes to this protective response because it is a plasma membrane receptor protein that recognizes amyloid and other molecules associated with neuronal debris, such as DNA, lipoproteins, and phospholipids. TREM2 loss-of-function genetic variant, R47H, decreases protective microglial capacity in Alzheimer's mouse models and in turn enables pathological amyloid diffusion and increased axonal dystrophy.

### Examination of TREM2 in Ocular Associated Cells

Previous studies have identified active roles of microglia in the pathogenesis of photoreceptor degeneration in retinitis pigmentosa. However, the contribution of microglia to photoreceptor degeneration is not clear, partly due to limited knowledge of microglial phenotypes during Retinitis pigmentosa (He J, Fu Y, Ge L, et al. Disease-associated microglial activation prevents photoreceptor degeneration by suppressing the accumulation of cell debris and neutrophils in degenerating rat retinas. Theranostics. 2022; 12(6):2687-2706. Published 2022 Mar. 6. doi:10.7150/thno.67954). The photoreceptor layer of cells adjacent to the RPE (i.e., the subretinal space) is shown herein in mice to contain protective microglia, and by single cell RNA-seq we found that these neuroprotective microglia had a disease-associated microglia (DAM)-like profile known in the art to be present and protective in mouse models of Alzheimer's disease. Loss-of-function experiments targeting these microglia in mouse models revealed increased damage to the photoreceptor layers and massive dysmorphology of the RPE. Hence our collective findings revealed a protective microglia population in mouse, akin to the ones identified in Alzheimer's models, that restricts disease progression in AMD.

### Gain of Function

The following experiments provide evidence of increased protection of photoreceptors and RPE in a model of outer retinal degenerative disease. Separately, we now have single cell RNA-seq and/or histological evidence that this protective population is present in at least six different mouse models of retinal degeneration. There is also have evidence from human postmortem AMD eyes that this microglia population is conserved in patients. Hence, these microglia represent a general response in photoreceptor/outer retinal degenerative diseases that can be therapeutically bolstered to restrict disease progression. Importantly, these microglia not only protect the photoreceptors, but also shield the RPE—the major pathological target tissue of geographic atrophy in AMD.

### Mouse TREM2 Agonistic Antibody

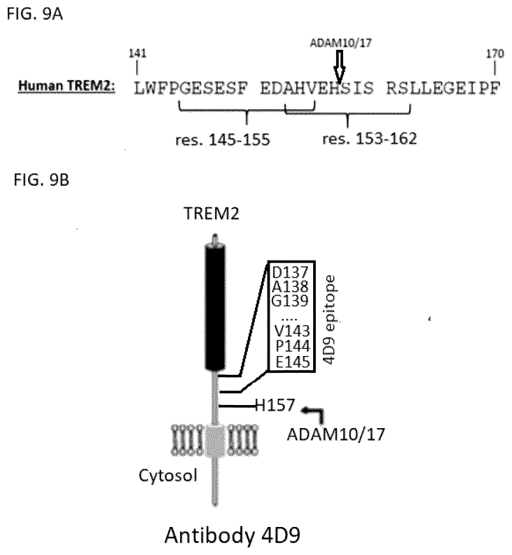

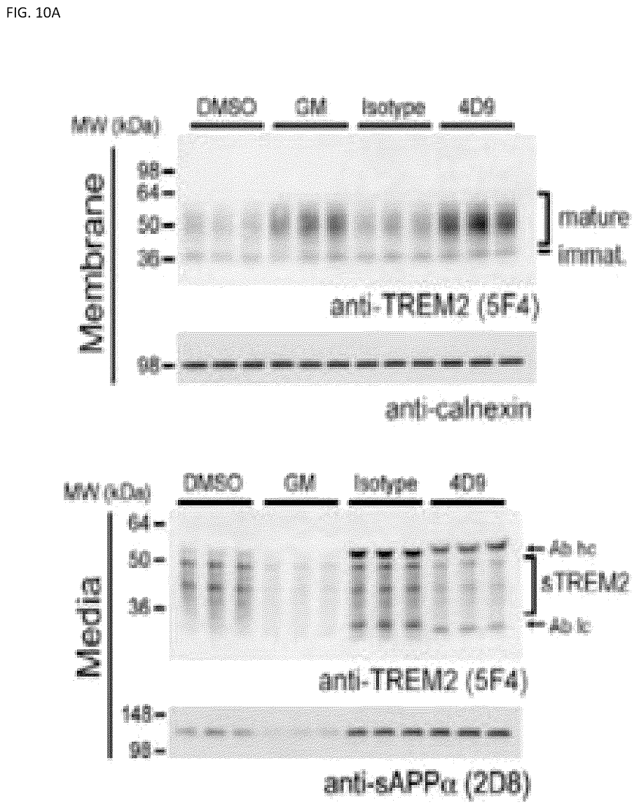

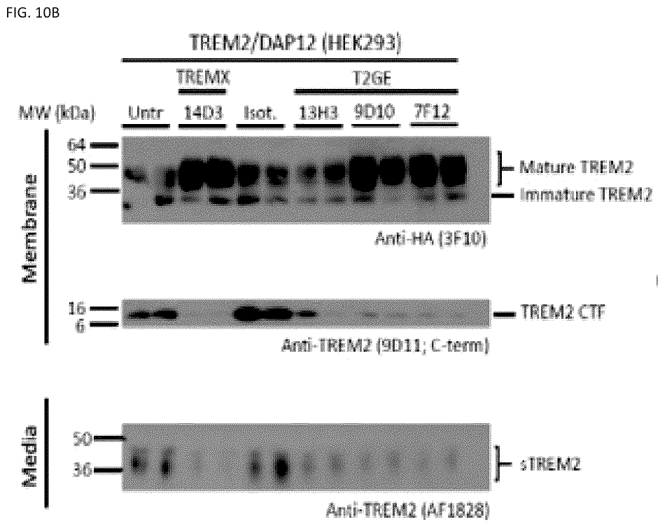

Antibody 4D9 is a mouse Trem2 agonistic antibody which ameliorates amyloid pathology in an AD mouse model. Further data has been generated by the inventors that show in a retinal degeneration model, antibody 4D9 rescues pathology as well and its epitope is depicted in FIG. 9B.

### Screening Assay

Described herein is an assay to screen for inhibitors of cleavage of human TREM2 on the surface of microglial cells of the human retina in the region of the TREM2 protein having the amino acid sequence of SEQ ID NO: 1. Preferably the assay screens for inhibitors of cleavage of human TREM2 on retinal microglial cells between histidine 157 and serine 158 residues, the site at which the ADAM10 and/or ADAM17 proteases of the ADAM (a disintegrin and metalloproteinase domain containing protein) family cleave human TREM2 on retinal microglial cells. Preferably the assay screens for inhibitors of cleavage of human TREM2 on retinal microglial cells between its histidine 157 and serine 158 residues where the cleavage is mediated by ADAM 10/17 proteases by selectively blocking access of ADAM proteases to the cleavage site between histidine 157 and serine 158 of human TREM2 on retinal microglial cells.

Inhibitors of cleavage of human TREM2 between its histidine 157 and serine 158 residues on retinal microglial cells reduce the amount of TREM2 shedding from the surface of microglial cells. Consistent with reduced retinal microglial cell surface TREM2, biological functions of TREM2 on retinal microglial cells, such as lipid sensing, ApoE binding, and phagocytosis are all decreased.

Thus, the screening assay described herein may open the opportunity to therapeutically modulate TREM2 function on retinal microglial cells by selectively blocking access of ADAM proteases to the cleavage site between histidine 157 and serine 158, as opposed to blocking ADAM 10/17 activity with conventional protease inhibitors, which inhibits numerous ADAM10/17 substrates.

TREM2 is exclusively expressed in microglia and is functionally required e.g. in phagocytosis of cellular debris [Colonna, M. and Y. Wang, TREM2 variants: new keys to decipher Alzheimer disease pathogenesis. Nat Rev Neurosci, 2016. 17(4): p. 201-7; Ulrich, J. D. and D. M. Holtzman, TREM2 Function in Alzheimer's Disease and Neurodegeneration. ACS Chem Neurosci, 2016. 7(4): p. 420-7]. Structurally, TREM2 is a type-1 membrane protein that is shuttled to the plasma membrane [Prada, I., et al., Triggering receptor expressed in myeloid cells 2 (TREM2) trafficking in microglial cells: continuous shuttling to and from the plasma membrane regulated by cell stimulation. Neuroscience, 2006. 140(4): p. 1139-48] where it may exert its biological functions. TREM2 undergoes regulated intramembrane proteolysis (RIP) [Kleinberger, G., et al.]. TREM2 mutations implicated in neurodegeneration impair cell surface transport and phagocytosis. Sci Transl Med, 2014. 6(243): p. 243ra86; Wunderlich, P., et al., Sequential proteolytic processing of the triggering receptor expressed on myeloid cells-2 (TREM2) protein by ectodomain shedding and gamma-secretase-dependent intramembranous cleavage. J Biol Chem, 2013. 288(46): p. 33027-36].

RIP is initiated on the cell surface by shedding of full-length TREM2 by metalloproteinases including ADAM10 and ADAM17 (disintegrin and metalloproteinase domain containing proteins). Shedding results in the secretion of soluble TREM2 (sTREM2). The membrane retained C-terminal fragment (CTF) is subsequently cleared by an intramembraneous cleavage by 7-secretase (see, e.g. Kleinberger et al. [Kleinberger, G., et al., TREM2 mutations implicated in neurodegeneration impair cell surface transport and phagocytosis. Sci Transl Med, 2014. 6(243): p. 243) [Wunderlich, P., et al., Sequential proteolytic processing of the triggering receptor expressed on myeloid cells-2 (TREM2) protein by ectodomain shedding and gamma-secretase-dependent intramembranous cleavage. J Biol Chem, 2013. 288(46): p. 33027-36, Glebov, K., et al., Functional involvement of gamma-secretase in signaling of the triggering receptor expressed on myeloid cells-2 (TREM2). J Neuroinflammation, 2016. 13: p. 17].

As cleavage of TREM2 ectodomain on retinal microglial cells occurs C-terminal to the histidine at position 157 (His157) of the amino acid sequence of TREM2, the assay described herein screens for a molecule which binds to human TREM2 on retinal microglial cells in the region of TREM2 defined by positions 141-170 and which blocks cleavage on the surface of retinal microglial cells of the TREM2 ectodomain C-terminal to His157. Therefore, the molecule may bind specifically to retinal microglial cells at any one or more of the amino acids at positions 141-170 of human TREM2, e.g. as shown in SEQ ID NO: 1. Such a molecule stabilizes or increases the amount of retinal microglial cell surface-bound TREM2, thereby preserving and stimulating activity of retinal microglial cells.

Thus, the instantly described screening assay provides a molecule that binds to human TREM2 on retinal microglial cells and reduces, (preferably inhibits and prevents) TREM2 cleavage. The reduction TREM2 cleavage can range anywhere from 1% to 100% reduction relative to wild type TREM2 cleavage. More specifically, in the context of the present invention cleavage (i.e. shedding) of the TREM2 ectodomain from retinal microglial cells is inhibited anywhere from 1% to 100% by the binding molecule of the present invention.

The screening assay described herein provides a molecule that can bind to a conformational binding site or to a linear binding site within positions 141-170 of the amino acid sequence of human TREM2 on retinal microglial cells. If the binding molecule is an antibody, these binding sites are called conformational epitope and linear epitope, respectively. A conformational binding site is composed of a discontinuous section of the amino acid sequence of TREM2. Such a binding site interacts with the binding molecule based on the 3-D structure surface feature, i.e. the tertiary structure of TREM2.

By contrast, linear binding sites interact with the binding molecule based on the primary structure of the amino acid sequence of human TREM2. Thus, a linear binding site is formed by a continuous sequence of amino acids of human TREM2 on retinal microglial cells. For example, the binding site of the herein provided binding molecule within human TREM2 on retinal microglial cells may comprise or overlap with any one of the polypeptides consisting of one or more of the amino acids at positions between and including 141-170. However, also smaller binding sites of 3-5 amino acids may be used by the herein provided binding molecule. For example, the binding site of the herein provided binding molecule within human TREM2 on retinal microglial cells may comprise or overlap with any one of the polypeptides consisting of one or more of the amino acids at positions 145-155 or 153-162. As mentioned above, it is prioritized that a binding molecule of the present invention binds to human TREM2 on retinal microglial cells in the region defined by positions between and including residues 141-170 of human TREM2 and inhibits cleavage of human TREM2 on retinal microglial cells between histidine 157 and serine 158 of human TREM2 by ADAM10 and/or ADAM17, preferably of membrane bound human TREM2 on retinal microglial cells.

There are several assays known in the art that can be used in order to quantify cleavage of TREM2 on microglial cells of human retina. These methods may be modified in the context of the present invention in order to assay (i.e. quantify) inhibition of TREM2 cleavage on human retinal microglial cells by the herein provided binding molecule. For example, inhibition of TREM2 cleavage on human retinal microglial cells can be tested (particularly quantified) by modifying the following assays that are known in the art, and described, e.g., in Kleinberger et al. [Kleinberger, G., et al., TREM2 mutations implicated in neurodegeneration impair cell surface transport and phagocytosis. Sci Transl Med, 2014. 6(243): p. 243]:
1.) Immunoblotting of membrane fractions or protein lysates of human and/or mouse TREM2 expressing cells, preferably retinal human microglial cells. Efficiency of TREM2 cleavage can be tested by analyzing higher molecular weight (“mature”) bands (see, e.g., Kleinberger et al. [Kleinberger, G., et al., TREM2 mutations implicated in neurodegeneration impair cell surface transport and phagocytosis. Sci Transl Med, 2014. 6(243): p. 243ra86] and Jonsson, T., et al., Variant of TREM2 associated with the risk of Alzheimer's disease. N Engl J Med, 2013. 368(2): p. 107-16).
2.) Immunoblotting of supernatants from human and/or mouse TREM2 expressing cells (see, e.g., [Kleinberger et al.], preferably retinal human microglial cells.
3.) ELISA-based quantification of soluble TREM2 in supernatants from human and/or mouse TREM2 expressing cells, preferably retinal human microglial cells, (see, e.g., [Kleinberger, G., et al., TREM2 mutations implicated in neurodegeneration impair cell surface transport and phagocytosis. Sci Transl Med, 2014. 6(243): p. 243ra86],
4.) Quantification of membrane-bound, preferably on retinal human microglial cell surface, (i.e. cell surface-exposed TREM2 by a surface biotinylation assay (see, e.g., Kleinberger et al.,).
5.) Quantification of membrane-bound, preferably on retinal human microglial cell surface membrane, (i.e. cell surface-exposed TREM2 on human and/or mouse cell lines and primary cells by flow-cytometry.
6.) Quantification of membrane-bound (i.e. cell surface-exposed TREM2 exposed on human and/or mouse cell lines and primary cells by cell-based ELISA technique, preferably on retinal human microglial cell surface membrane.
7.) Quantification of cell surface (i.e. membrane)-exposed TREM2 on human and/or mouse cell lines and primary cells by surface immunocytochemistry (see e.g. [Wunderlich, P., et al., Sequential proteolytic processing of the triggering receptor expressed on myeloid cells-2 (TREM2) protein by ectodomain shedding and gamma-secretase-dependent intramembranous cleavage. J Biol Chem, 2013. 288(46): p. 33027-36]), preferably on retinal human microglial cell surface.
8.) ELISA-based quantification of soluble TREM2 (sTREM2) from tissue and/or biofluids of human and/or mouse (e.g. from eye, brain, liver, spleen, serum, plasma, cerebrospinal fluid and/or urine), preferably from retinal human microglial cells.
9.) Immunoblotting of TREM2 from tissue and/or biofluids from human and/or mouse origin (e.g. from eye, brain, liver, spleen, serum, plasma, cerebrospinal fluid and/or urine), preferably on retinal human microglial cells.

Any one of the above described methods may be used in the context of the present invention for testing whether a particular binding molecule inhibits TREM2 cleavage on human retinal microglial cells. Thus, one aspect of the present invention relates to the herein provided binding molecule, wherein inhibition of TREM2 cleavage on human retinal microglial cells is assayed by immunoblotting, ELISA-based quantification of soluble TREM2, quantification of surface-bound TREM2 by surface biotinylation assays, quantification of surface-bound TREM2 by flow-cytometry, quantification of surface-bound TREM2 by surface immunocytochemistry and/or quantification of surface-bound TREM2 by cell-based ELISA technique.

The inhibition of cleavage of TREM2 on human retinal microglial cells by a binding molecule correlates with the amount of membrane bound TREM2. Thus, the amount of membrane bound TREM2 on retinal microglial cells is increased in the presence of the binding molecule as compared to the amount of membrane bound TREM2 on retinal microglial cells in the absence of the binding molecule. For example, a binding molecule may be considered as a binding molecule that inhibits cleavage of TREM2 on retinal microglial cells, if in the presence of said binding molecule the amount of membrane bound TREM2 is at least 110%, preferably at least 120%, more preferably at least 150%, even more preferably at least 200%, and even more preferably at least 250% of the amount of membrane bound TREM2 in the absence of the binding molecule, as assayed, e.g., by any one of the assays mentioned above, particularly by immunoblotting or flow-cytometry. In such an assay cells may be used that comprise a TREM2 cleavage enzyme.

Accordingly, a binding molecule may be considered as a binding molecule that inhibits cleavage of TREM2 on retinal microglial cells, if in the presence of said binding molecule the amount of membrane bound TREM2 on retinal microglial cells is at least 10%, preferably at least 20%, more preferably at least 50%, even more preferably at least 100%, and even more preferably at least 150% more than the amount of membrane bound TREM2 on retinal microglial cells in the absence of the binding molecule, as assayed, e.g., by any one of the assays mentioned above, particularly by immunoblotting or flow-cytometry. As mentioned, any one of the assays mentioned above, particularly immunoblotting or ELISA based quantification of sTREM2 may be used for quantifying inhibition of TREM2 cleavage on retinal microglial cells by the herein provided binding molecule. In such an assay cells may be used that comprise a TREM2 cleavage enzyme.

Herein, the term “membrane bound TREM2” (also called “membrane-bound TREM2”) means that the full-length TREM2 protein, including its ectodomain, is glycosylated and bound to a membrane, particularly to the plasma membrane of microglia cells.

The degree of inhibition of cleavage of TREM2 on retinal microglial cells by a binding molecule negatively correlates with the amount of soluble TREM2 (sTREM2) released from retinal microglial cells in the presence of the binding molecule as compared to the amount of sTREM2 released from retinal microglial cells in the absence of the binding molecule. For example, a binding molecule may be considered as a binding molecule that inhibits cleavage of TREM2 on retinal microglial cells, if in the presence of said binding molecule the amount of sTREM2 is 0-90%, preferably 0-80%, more preferably 0-70%, even more preferably 0-60%, even more preferably 0-50% and even more preferable 0-20% of the amount of sTREM2 in the absence of the binding molecule, as assayed, e.g., by any one of the assays mentioned above, particularly by ELISA-based quantification of sTREM2.

The Examples show that the antibody clones of 7F12, 13F4, 9D10 and 14B3 decreases TREM2 cleavage by about 70% while increasing the level of mature TREM2 by up to five-fold. Thus, it is preferred that, as compared to non-treated cells (i.e. cells that do not comprise the binding molecule of the invention), the binding molecule decreases TREM2 cleavage by at least 60% or most preferably by about 70%. The amount of TREM2 cleavage may be assayed by ELISA, which for quantification is a more robust assay than quantification of Western Blot results.

By inhibiting TREM2 cleavage on retinal microglial cells, the disclosed TREM2 binding antibodies and other molecules obtained using the screening methods described herein preserves and/or stimulates activity of microglia cells, and/or the activity of other TREM2 expressing cells, preferably, the preserving and/or stimulating activity of microglia cells. Said activity of microglia cells, preferably retinal microglial cells, may be phagocytosis activity, migration, calcium signaling, Syk activation, and/or proliferation. TREM2 is also regulating the inflammatory cytokine production and survival of microglia cells, preferably human retinal microglial cells. Therefore, said activity of microglia cells may also be regulation of inflammatory cytokine production and/or survival.

Treatment of Degenerative and Vascular Disease of the Retina and/or Posterior Segment of the Eye

The invention includes methods for treating a degenerative and vascular disease of the retina and/or posterior segment of the eye in a subject by administering to a subject an effective amount of an antibody or fragment thereof that binds to an epitope of SEQ ID NO:1, preferably an epitope of GESESFEDAHV (SEQ ID NO:2) of SEQ ID NO: 1.

### Retinal Diseases for Treatment

Retinal diseases which may be treated with an antibody as disclosed herein are preferably selected from the group consisting of retinitis pigmentosa (RP), including syndromic and non-syndromic forms, X-chromosome linked, recessive and dominant forms, rod-cone dystrophies, Usher's syndrome, Stargardt's disease, cone-rod dystrophies, cone dystrophies, achromatopsia, blue cone monochromacy, enhanced S-cone syndrome, rod dystrophies, choroideremia, Leber's congenital amaurosis, juvenile X-chromosome linked retinoschisis (JXLR), Best disease, Gyrate atrophy, fundus albipunctatus, retinitis punctata albescens, fleck retina of Kandori, bietti crystalline retinal dystrophy, North Carolina macular dystrophy, fenestrated sheen macular dystrophy, central areolar choroidal dystrophy (CACD), adult-onset foveomacular vitelliform dystrophy, Batten's disease, familial dominant drusen, congenital stationary night blindness, familial exudative vitreoretinopathy (FEVR), ocular albinism, oculocutaneous albinism, fovea hypoplasia, retinopathy of prematurity, abetalipoproteinemia, Stickler syndrome, retinal dystrophy (Bothnia type), dry age-related macular degeneration (dry AMD), wet age-related macular degeneration (wet AMD), geographic atrophy (GA), myopic degeneration, polypoidal choroidal vasculopathy (PCV), crystalline maculopathy (drug-related, hyperoxaluria, cystinosis, Sjogren-Larsson syndrome), west African crystalline maculopathy, solar retinopathy, talc retinopathy, diabetic retinopathy, sickle cell retinopathy, central serious retinopathy, macular telangectasia, angioid streaks, eales disease, retinal detachment, retinal dialysis, peripheral retinoschisis, central/branch retinal artery occlusion (CRAO/BRAO), central/branch retinal vein occlusion (CRVO/BRVO), haemorrhagic occlusive retinal vasculitis (HORV), drug-induced maculopathies including chloroquine, hydroxychloroquine, phenothiazine, quinine sulfate, thioridazine, clofazimine, cholopromazine, deferoxamine, chloroquine-derivatives, cisplatin, carmustine, chlofazimine and vigabatrin; crystal-induced maculopathies including tamoxifen, talc, canthaxanthine, methoxyflurane and nitrofurantoin; cystoid macular edema (CME) including epinephrine, latanoprost, nicotinic acid; progressive outer retinal necrosis (PORN), acute retinal necrosis (ARN), CMV-retinitis, Sarcoidosis, acute syphilitic posterior placoid chorioretinitis, tuberculosis chorioretinitis, toxoplasmic retinochoroiditis, Vogt-Koyanagi-Harada (VKH), posterior Uveitis and retinal vasculitis, intermediate uveitis, pars planitis+/−CME, enophthalmitis (anterior and/or posterior), posterior scleritis, masquerade syndromes, acute posterior multifocal placoid pigment epitheliopathy (APMPPE), relentless placoid chorioretinopathy (RPC), serpiginous choroiditis, multiple evanescence white dot syndrome (MEWDS), multifocal choroiditis and panuveitis (MCP), punctate inner choroidopathy (PIC), birdshot retinochoroidopathy, presumed ocular histoplasmosis syndrome (POHS), acute macular neuroretinopathy (AMN) and acute zonal occult outer retinopathy (AZOOR).

Such antibodies are suitable for the use in the treatment of acquired degeneration selected from the group consisting of dry age-related macular degeneration (dry AMD), wet age-related macular degeneration (wet AMD), geographic atrophy (GA), myopic degeneration, polypoidal choroidal vasculopathy (PCV), crystalline maculopathy (drug-related, hyperoxaluria, cystinosis, Sjogren-Larsson syndrome), west African crystalline maculopathy, solar retinopathy, talc retinopathy, diabetic retinopathy, sickle cell retinopathy, central serious retinopathy, macular telangectasia, angioid streaks, eales disease, retinal detachment, retinal dialysis, peripheral retinoschisis.

An antibody as disclosed herein is suitable for the use in the treatment of vascular related retinal degeneration selected from the group consisting of central/branch retinal artery occlusion (CRAO/BRAO), central/branch retinal vein occlusion (CRVO/BRVO), haemorrhagic occlusive retinal vasculitis (HORV).

An antibody as disclosed herein is suitable for the use in the treatment of drug-induced maculopathies selected from the group consisting of chloroquine, hydroxychloroquine, phenothiazine, quinine sulfate, thioridazine, clofazimine, cholopromazine, deferoxamine, chloroquine-derivatives, cisplatin, carmustine, chlofazimine and vigabatrin as well as crystal-induced maculopathies including tamoxifen, talc, canthaxanthine, methoxyflurane, nitrofurantoin, cystoid macular edema (CME) including Epinephrine, latanoprost and nicotinic acid.

An antibody as disclosed herein is suitable for the use in the treatment of infectious and/or inflammatory eye diseases selected from the group consisting of progressive outer retinal necrosis (PORN), acute retinal necrosis (ARN), CMV-retinitis, Sarcoidosis, acute syphilitic posterior placoid chorioretinitis, tuberculosis chorioretinitis, toxoplasmic retinochoroiditis, Vogt-Koyanagi-Harada (VKH), posterior Uveitis and retinal vasculitis, intermediate uveitis, pars planitis+/−CME, enophthalmitis (anterior and/or posterior), posterior scleritis and masquerade syndromes.

An antibody as disclosed herein is suitable for the use in the treatment of white dot syndromes selected from the group consisting of acute posterior multifocal placoid pigment epitheliopathy (APMPPE), relentless placoid chorioretinopathy (RPC), serpiginous choroiditis, multiple evanescence white dot syndrome (MEWDS), multifocal choroiditis and panuveitis (MCP), punctate inner choroidopathy (PIC), birdshot retinochoroidopathy, presumed ocular histoplasmosis syndrome (POHS), acute macular neuroretinopathy (AMN) and acute zonal occult outer retinopathy (AZOOR).

### Pharmaceutical Compositions

Pharmaceutical compositions for use in accordance with the present disclosure may be formulated with one or more appropriate carriers, excipients or diluents. The composition (e.g., an agent capable of bolstering neuroprotective microglial cells [e.g., a Trem2 agonist] and one or more appropriate carriers, excipients, or diluents) may optionally include one or more additional compounds (e.g., an additional therapeutic agent as provided herein). The term “carrier” refers to a diluent, adjuvant, excipient, or vehicle with which the active agent is administered. The carriers in the pharmaceutical composition may comprise a binder, such as microcrystalline cellulose, polyvinylpyrrolidone (polyvidone or povidone), gum tragacanth, gelatin, starch, lactose or lactose monohydrate; a disintegrating agent, such as alginic acid, maize starch and the like; a lubricant or surfactant, such as magnesium stearate, or sodium lauryl sulphate; and a glidant, such as colloidal silicon dioxide. The carrier(s) must be “acceptable” in the sense of being compatible with the other ingredients of the composition and not deleterious to the recipient thereof. The exact nature of the carrier, excipient or diluent will depend upon the desired use for the composition and may range from being suitable or acceptable for veterinary uses to being suitable or acceptable for human use.

The following exemplification of carriers, modes of administration, dosage forms, etc., are listed as known possibilities from which the carriers, modes of administration, dosage forms, etc., may be selected for use with the present disclosure. Those of ordinary skill in the art will understand, however, that any given formulation and mode of administration selected should first be tested to determine that it achieves the desired results.

When used to treat or prevent a disease, such as an eye disease, the compounds described herein may be administered singly, as mixtures of one or more compounds or in mixture or combination with other agents (e.g., one or more additional therapeutic agents) useful for treating such diseases and/or the symptoms associated with such diseases. Such agents may include, but are not limited to, anti-VGEF compounds, phototherapy, microglial checkpoint inhibitors as described herein, to name a few. The compounds may be administered in the form of compounds per se, or as pharmaceutical compositions comprising a compound.

Pharmaceutical compositions comprising the compound(s) may be manufactured by means of conventional mixing, dissolving, granulating, dragee-making, levigating, emulsifying, encapsulating, entrapping or lyophilization processes. The compositions may be formulated in conventional manner using one or more physiologically acceptable carriers, diluents, excipients or auxiliaries which facilitate processing of the compounds into preparations which can be used pharmaceutically.

The compounds may be formulated in the pharmaceutical composition per se, or in the form of a hydrate, solvate, N-oxide or pharmaceutically acceptable salt, as previously described. Typically, such salts are more soluble in aqueous solutions than the corresponding free acids and bases, but salts having lower solubility than the corresponding free acids and bases may also be formed.

### Mode of Administration

Pharmaceutical compositions may take a form suitable for virtually any mode of administration, including, for example, topical, ocular, oral, buccal, systemic, nasal, injection (e.g., systemic), transdermal, rectal, vaginal, etc., or a form suitable for administration by inhalation or insufflation.

An antibody disclosed herein can be delivered to the eye through a variety of routes, including but not limited to topical application to the eye or by intraocular injection into, for example, the vitreous or subretinal (interphotoreceptor) space; locally by insertion or injection into the tissue surrounding the eye; systemically through an oral route or by subcutaneous, intravenous or intramuscular injection; or via catheter or implant. Most preferably, the compound of the present application is delivered by intraocular injection. The antibody can be administered prior to the onset of the condition to prevent its occurrence, such as during eye surgery, immediately after the onset of the pathological condition, or during the occurrence of an acute or protracted condition.

For topical administration, the compound(s) may be formulated as solutions, gels, ointments, creams, suspensions, etc. as are well-known in the art.

Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen free water, buffer, dextrose solution, etc., before use. To this end, the active compound(s) may be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pre-gelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, Cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the compound, as is well known. For buccal administration, the compositions may take the form of tablets or lozenges formulated in conventional manner. For rectal and vaginal routes of administration, the compound(s) may be formulated as solutions (for retention enemas) suppositories or ointments containing conventional suppository bases such as cocoa butter or other glycerides.

For nasal administration or administration by inhalation or insufflation, the compound(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For ocular administration, the compound(s) may be formulated as a solution, emulsion, suspension, etc. suitable for administration to the eye. A variety of vehicles suitable for administering compounds to the eye are known in the art.

For prolonged delivery, the compound(s) can be formulated as a depot preparation for administration by implantation or intramuscular injection. The compound(s) may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the compound(s) for percutaneous absorption may be used. To this end, permeation enhancers may be used to facilitate transdermal penetration of the compound(s).

Alternatively, other pharmaceutical delivery systems may be employed. Liposomes and emulsions are well-known examples of delivery vehicles that may be used to deliver compound(s). Certain organic solvents such as dimethyl sulfoxide (DMSO) may also be employed, although usually at the cost of greater toxicity.

The pharmaceutical compositions may, if desired, be presented in a pack or dispenser device which may contain one or more unit dosage forms containing the compound(s). The pack may, for example, comprise metal or plastic foil, such as a blister pack. The pack or dispenser device may be accompanied by instructions for administration.

### Dosage

The compound(s) described herein, or compositions thereof, will generally be used in an amount effective to achieve the intended result, for example in an amount effective to treat or prevent the particular disease being treated (e.g., an eye disease). By therapeutic benefit is meant eradication or amelioration of the underlying disease/disorder being treated and/or eradication or amelioration of one or more of the symptoms associated with the underlying disease/disorder such that the patient reports an improvement in feeling or condition, notwithstanding that the patient may still be afflicted with the underlying disease/disorder. Therapeutic benefit also generally includes halting or slowing the progression of the disease, regardless of whether improvement is realized.

The amount of compound(s) administered will depend upon a variety of factors, including, for example, the particular indication being treated, the mode of administration, whether the desired benefit is prophylactic or therapeutic, the severity of the indication being treated and the age and weight of the patient, the bioavailability of the particular compound(s) the conversation rate and efficiency into active drug compound under the selected route of administration, etc.

Determination of an effective dosage of compound(s) for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration of the metabolite active compound that is at or above an IC50 of the particular compound as measured in as in vitro assay. Calculating dosages to achieve such circulating blood or serum concentrations taking into account the bioavailability of the particular compound via the desired route of administration is well within the capabilities of skilled artisans. Initial dosages of compound can also be estimated from in vivo data, such as animal models. Animal models useful for testing the efficacy of the active metabolites to treat or prevent the various diseases described above are well-known in the art. Animal models suitable for testing the bioavailability and/or metabolism of compounds into active metabolites are also well-known. Ordinarily skilled artisans can routinely adapt such information to determine dosages of particular compounds suitable for human administration.

Dosage amounts will typically be in the range of from about 0.0001 mg/kg/day, 0.001 mg/kg/day or 0.01 mg/kg/day to about 100 mg/kg/day, but may be higher or lower, depending upon, among other factors, the activity of the active compound, the bioavailability of the compound, its metabolism kinetics and other pharmacokinetic properties, the mode of administration and various other factors, discussed above. Dosage amount and interval may be adjusted individually to provide plasma levels of the compound(s) and/or active metabolite compound(s) which are sufficient to maintain therapeutic or prophylactic effect. For example, the compounds may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective dosages without undue experimentation.

Preferably, the herein provided binding molecule preserves and/or stabilizes phagocytosis activity of microglia cells. There are several assays known in the art that can be used for measuring phagocytosis activity of cells. For example, for testing whether a given binding molecule preserves and/or stabilizes phagocytosis activity of retinal microglial cells, phagocytosis can be tested as described in Kleinberger et al. and Xiang et al. [Xiang, X., et al., TREM2 deficiency reduces the efficacy of immunotherapeutic amyloid clearance. EMBO Mol Med, 2016]. An increase in the phagocytosis activity on retinal microglial cells can be tested in vivo using methods described in [Heneka, M. T., et al., NLRP3 is activated in Alzheimer's disease and contributes to pathology in APP/PS1 mice. Nature, 2013. 493(7434): p. 674-8]. For testing whether a given binding molecule preserves and/or stabilizes calcium signaling of retinal microglial cells several means and methods are known in the art, and described, e.g., in [Glebov, K., et al., Functional involvement of gamma-secretase in signaling of the triggering receptor expressed on myeloid cells-2 (TREM2). J Neuroinflammation, 2016. 13: p. 17].

Treatment with Additional Therapeutic Agent

In another embodiment, the method further comprises administering to the subject at least one additional therapeutic agent. In some embodiments, the at least one additional therapeutic agent comprises one or more microglia checkpoint molecule inhibitor. Such agents are able to cause an increase in the number of neuroprotective microglia by releasing or circumventing a restraint imposed on microglia immune activity by at least one microglia checkpoint molecule. Such microglia checkpoint molecules include, but are not limited to, Cx3cr1, CD200, CD200R, P2ry12, P2ry13, Ccr5, Tmem119, Calm2, Cd164, Cmtm6, Crybb, Ecscr, Fscn1, Glu1, Gpr56, Ifngr1, Lpcat2, Lrba, Lyn, Maf, Marcks, Olfm13, Pmepa1, Ptgs1, Rhob, Slco2b1, Selplg, Serinc3, Sparc, Srgap2, Txnip, Zfhx3 and the like. Blocking these microglia checkpoint molecules, at the expression or at the activity level, is expected to enhance the neuroprotective microglial phenotype thereby increasing the number of neuroprotective microglial cells. Blocking at the activity level can be done by a binding molecule such as an antagonist to the checkpoint molecules, for example by an antibody to, or small molecule inhibiting, said molecules. The antibody or other antagonist binding molecule blocks or inactivates, or otherwise neutralizes the activity of the checkpoint molecule to stop or attenuate its effect, thereby causing the cells to progress to the neuroprotective microglial phenotype.

It is noted that some of the above-listed microglia checkpoint molecules have partners such that their effect is achieved by complexing with the partners. Accordingly, blocking of activity or inhibiting the expression of the partner protein will have a similar effect as blocking activity or inhibiting expression of the microglia checkpoint protein. Examples for such proteins are CD200R (partner of CD200), and Cx3cL1 (partner of Cx3cr1). Accordingly, blocking the activity or reducing the expression of partners of the above-listed microglia checkpoint molecules is also within the scope of the present disclosure.

In some embodiments, the at least one additional therapeutic agent releases a restraint imposed on said microglia by blocking, inhibiting, or attenuating the activity of the at least one microglia checkpoint molecule as provided herein.

### Antibody Production

Various procedures are known in the art and may be used for the production of such antibodies and/or fragments (see, for example, [Harlow, Antibodies, A Laboratory Manual. 1988: Cold Spring Harbor Publications, New York]. Herein the abbreviations “VL”, “VH”, “CL” and “CH” refer to variable domain of the antibody light chain, variable domain of the antibody heavy chain, constant domain of the antibody light chain and constant domain of the antibody heavy chain, respectively.

For preparation of monoclonal antibodies, several techniques which provide antibodies by continuous cell culture can be used. Examples include the hybridoma technique [Kohler, G. and C. Milstein, Continuous cultures of fused cells secreting antibody of predefined specificity. Nature, 1975. 256(5517): p. 495-7], the human B-cell hybridoma technique [Kozbor, D. and J. C. Roder, The production of monoclonal antibodies from human lymphocytes. Immunol Today, 1983. 4(3): p. 72-9] and the EBV-hybridoma technique [Cole, Monoclonal Antibodies and Cancer Therapy. 1985: Alan R. Liss, Inc.]. For example, the monoclonal antibodies to be used in accordance with the present invention may be made by the hybridoma method first described by Kohler, G. and C. Milstein, Continuous cultures of fused cells secreting antibody of predefined specificity. Nature, 1975. 256(5517): p. 495-7, or may be made by recombinant methods, e.g., as described in U.S. Pat. No. 4,816,567. The monoclonal antibodies for use with the present invention may also be isolated from phage antibody libraries, e.g. using the techniques described in Clackson, T., et al., Making antibody fragments using phage display libraries. Nature, 1991. 352(6336): p. 624-8.; as well as in Marks, J. D., et al., By-passing immunization. Human antibodies from V-gene libraries displayed on phage. J Mol Biol, 1991. 222(3): p. 581-97.

Antibody fragments can be prepared, for example, by recombinant techniques or enzymatic or chemical cleavage of intact antibodies. For producing a single chain Fv (scFv) the two domains of the Fv fragment, VL and VH, can be joined, using recombinant methods, by a synthetic linker that enables them to be made as a single protein chain in which the VL and VH regions pair to form a monovalent molecule (see, e.g., Bird et al. [Bird, R. E., et al., Single-chain antigen-binding proteins. Science, 1988. 242(4877): p. 423-6] and [Huston, J. S., et al., Protein engineering of antibody binding sites: recovery of specific activity in an anti-digoxin single-chain Fv analogue produced in Escherichia coli. Proc Natl Acad Sci USA, 1988. 85(16): p. 5879-83]. Further techniques for the production of single chain antibodies are described, e.g., [Pluckthun, The Pharmacology of Monoclonal Antibodies. Vol. 113. 1994: Rosenburg and Moore eds. Springer-Verlag, N.Y.] and U.S. Pat. No. 4,946,778.

Methods of immunization, producing and isolating antibodies (polyclonal and monoclonal) are known to those skilled in the art and described in the scientific and patent literature, (see, e.g., [Coligan, Current Protocols in Immunology. 1991: Wiley/Greene, N.Y.; Stites, Basic and Clinical Immunology. 7th ed ed.: Lange Medical Publications, Los Altos, Calif.; Goding, Monoclonal Antibodies: Principles and Practice. 2d ed. 1986: Academic Press, New York, N.Y.; Kohler, G. and C. Milstein, Continuous cultures of fused cells secreting antibody of predefined specificity. Nature, 1975. 256(5517): p. 495-7]). Antibodies can also be generated in vitro, e.g., by using a recombinant antibody binding site expressing phage display library; in addition or alternatively to the traditional in vivo methods using animals (see, e.g., Hoogenboom, H. R., Designing and optimizing library selection strategies for generating high-affinity antibodies. Trends Biotechnol, 1997. 15(2): p. 62-70 and Katz, B. A., Structural and mechanistic determinants of affinity and specificity of ligands discovered or engineered by phage display. Annu Rev Biophys Biomol Struct, 1997. 26: p. 27-45.

### Screening Assays for Antibodies

Generation and selection of monoclonal antibodies against the human TREM2 cleavage site may be performed as follows. A peptide comprising the human TREM2 cleavage site (e.g. a peptide comprising or comprised within the amino acid sequence of SEQ ID NO: 1 or fragment thereof, is coupled at the N-terminus to ovalbumin (OVA). Non-human animals such as mice or rats may be immunized with the OVA-coupled peptide and incomplete Freund's adjuvant. After 6 weeks, a boost without incomplete Freund's adjuvant may be given 3 days before fusion. Fusion of the myeloma cell line P3X63-Ag8.653 with the immune spleen cells may be performed using polyethylene glycol 1500 according to standard procedure (Koehler and Milstein, Nature. 1975, 256:495-497). Hybridoma supernatants may be tested for binding to the peptide immunogen (e.g. a peptide which is SEQ ID NO: 1 itself or a peptide fragment thereof in an enzyme-linked immunoassay using a biotinylated version of the peptide bound to avidin-coated plates. Bound antibodies may be detected with antibodies against IgG isotypes, detectable label such as a fluorescent agent, an enzymatic label, or a radioisotope. Alternatively, binding of the antibody to the antigen may be detected by using a secondary antibody having such a detectable label thereon. Particular assays include ELISA assays, sandwich assays, radioimmunoassays, immunohistochemical methods and Western Blots.

Fully-human antibodies may be produced, for example, by phage display, which is a widely used screening technology that enables production and screening of fully-human antibodies. Accordingly, also phage antibodies can be used in context of this invention. Phage display methods are described, for example, in U.S. Pat. Nos. 5,403,484, 5,969,108 and 5,885,793. Another technology which enables development of fully-human antibodies involves a modification of mouse hybridoma technology. Mice are made transgenic to contain the human immunoglobulin locus in exchange for their own mouse genes (see, for example, U.S. Pat. No. 5,877,397). (Fully-)mouse or (Fully-)rat antibodies may be produced analogously.

Antibodies that Bind to Human TREM2 and Prevent TREM2 Cleavage

In the screening assay described herein, antibodies which bind to the human TREM2 at or near the cleavage site (e.g. to an epitope within the peptide of SEQ ID NO: 1, such as an epitope comprising His 157 and/or Ser158) and reduce or inhibit cleavage by ADAM10 and or ADAM17 are provided.

Human TREM2-reactive hybridoma supernatants are screened for their ability to detect TREM2 on the cell surface of HEK293 Flp-In cells stably overexpressing human wild-type TREM2. This procedure is described in Kleinberger, G., et al., TREM2 mutations implicated in neurodegeneration impair cell surface transport and phagocytosis. Sci Transl Med, 2014. 6(243): p. 243. In particular, HEK293 Flp-In cells either expressing human full-length TREM2 or empty vector (control) are incubated with the respective TREM2-reactive supernatants. Binding of TREM2-reactive supernatants is visualized using isotype-specific antibodies. A detailed description of the generation and selection of monoclonal antibodies against the TREM2 cleavage site is given in the Examples.

SEQ ID NO 1: Amino acid sequence of Human TREM2, at positions 141-170.

(SEQ ID NO: 1)
LWFPGESESFEDAHVEHSISRSLLEFEIPF

For purposes of clarity, and in no way limiting the scope of the teachings, unless otherwise indicated, all numbers expressing quantities, percentages or proportions, and other numerical values recited herein, should be interpreted as being preceded in all instances by the term “about.” Accordingly, the numerical parameters recited in the present specification are approximations that may vary depending on the desired outcome. For example, each numerical parameter may be construed in light of the number of reported significant digits and by applying ordinary rounding techniques.

The following Examples are provided by way of illustration and not by way of limitation.

### EXAMPLES

### Example 1

Screening Assay for Antibodies Specific for the human TREM2 Cleavage Site and which reduce or inhibit cleavage of human Trem2 by ADAM10 and or ADAM17 are provided.

Generation and Selection of Monoclonal Antibodies which bind to an epitope of SEQ ID NO:1 and which reduce or inhibit cleavage of human Trem2 by ADAM10 and/or ADAM17.

A peptide comprising the amino acid sequence of residues 145-155 (SEQ ID NO:2) of human TREM2 or residues 153-162 (SEQ ID NO:3) of human TREM2 was synthesized and coupled at the N-terminus to ovalbumin or biotin (Peps4LS, Heidelberg, Germany). Rats were immunized subcutaneously (s.c.) and intraperitoneally (i.p.) with a mixture of 50 μg OVA-coupled peptide in 500 l PBS, 5 nmol CpG2006 (TIB MOLBIOL, Berlin, Germany), and 500 l incomplete Freund's adjuvant. After 6 weeks, a boost without Freund's adjuvant was given i.p. and s.c. 3 days before fusion. Fusion of the myeloma cell line P3X63-Ag8.653 with the rat immune spleen cells was performed using polyethylene glycol 1500 according to standard procedure (Koehler and Milstein, Nature. 1975, 256:495-497). After fusion, the cells were plated in 96-well plates using RPMI 1640 with 20% fetal calf serum, penicillin/streptomycin, glutamine, pyruvate, and non-essential amino acids supplemented with HAT HybriMax medium supplement (Sigma,). Hybridoma supernatants were tested in an enzyme-linked immunoassay using biotinylated peptides (0.2 μg/ml) comprising the amino acid sequence of residues 145-155 of human TREM2 or residues 153-162 of human TREM2 bound to avidin-coated plates. After blocking with PBS/2% FCS, hybridoma supernatants were added for 30 min. After one wash with PBS, bound antibodies were detected with a cocktail of HRP-conjugated mAbs against the four rat IgG isotypes. HRP was visualized with ready to use TMB substrate (1-Step™ Ultra TMB-ELISA, Thermo).

The hybridoma cells of TREM2-reactive supernatants from antibody hybridomas capable of binding selectively both to an epitope of SEQ ID NO:1 and to TREM2 on the cell surface were cloned at least twice by limiting dilution. The IgG subclass was determined by an ELISA assay with mouse anti-rat kappa light chain antibodies as capture and HRP-coupled mouse anti-rat IgG subclass-specific antibodies for detection.

The amino acid sequences of the variable heavy chain and the variable light chain of some of the produced antibodies was determined.

Antibody clones were purified from hybridoma supernatants to be able to test different antibody concentrations in cell culture experiments. In particular, HEK cells stably overexpressing wt TREM2 were treated with three different antibody clones at a final concentration of 50 μg/mL for 24 hours. As a negative control we included a monoclonal antibody, which binds to the C terminus of TREM2 and should therefore not interfere with ectodomain shedding.

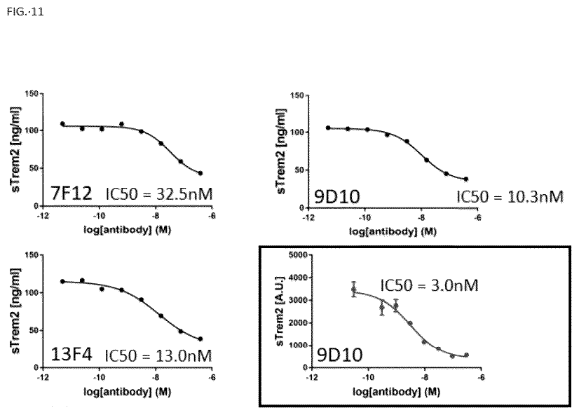

Levels of sTREM2 as shown in the bottom blot of FIG. 10B clearly show that selected antibody clones strongly reduce the extent of ectodomain cleavage. This is particularly evident for clones 13H3, 9D10 and 7F12 which secrete antibodies each of which falls into the group of “TGE” antibodies generated from rats immunized with the peptide “GESESFEDAHV” (SEQ ID NO:2), and for clone 14D3 which secrete antibodies each of which falls into the group of “TREMX” antibodies generated from rats immunized with the peptide “AHVEHSISRS” (SEQ ID NO:3).

As expected and shown in the top blot, inhibition of ectodomain cleavage leads to corresponding increases in levels of mature TREM2 in the membrane fraction, which for clones 13H3, 9D10 and 7F12 are comparable to if not higher than the increase as seen with the isotype control.

The western blot shows only the result of a single experiment, which is representative for the effects of most of the antibodies.

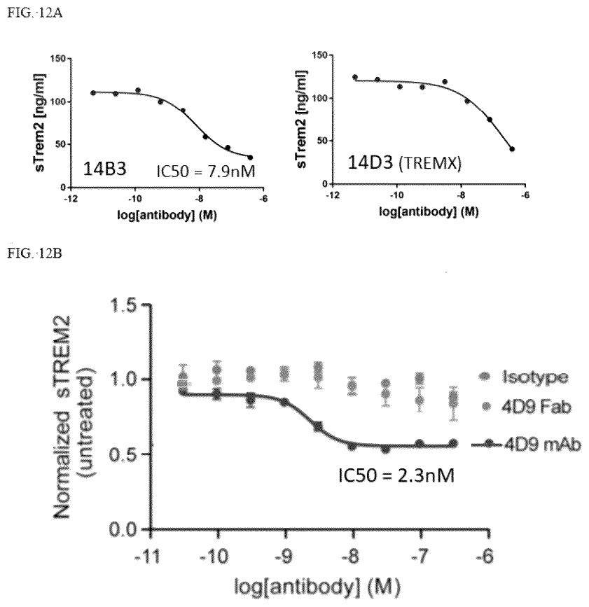

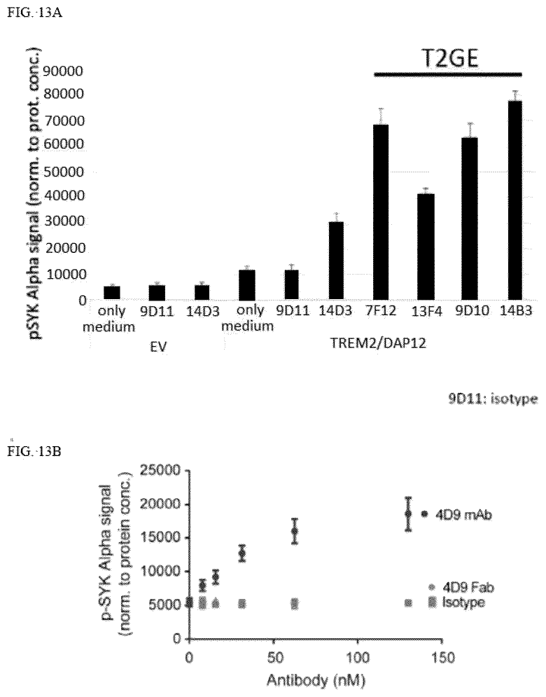

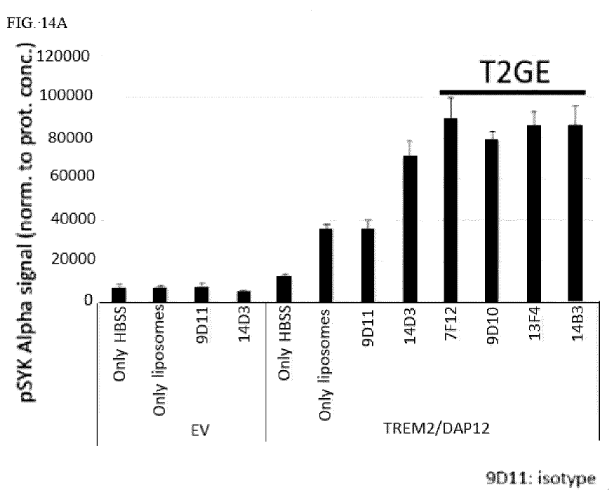

FIGS. 11 and 12A and 12B provide data indicating that several of the antibodies (7F12, 13F4, 9D10 and 14B3) obtained by the aforementioned screening assay inhibit TREM2 shedding with IC50s in the low nM regime.

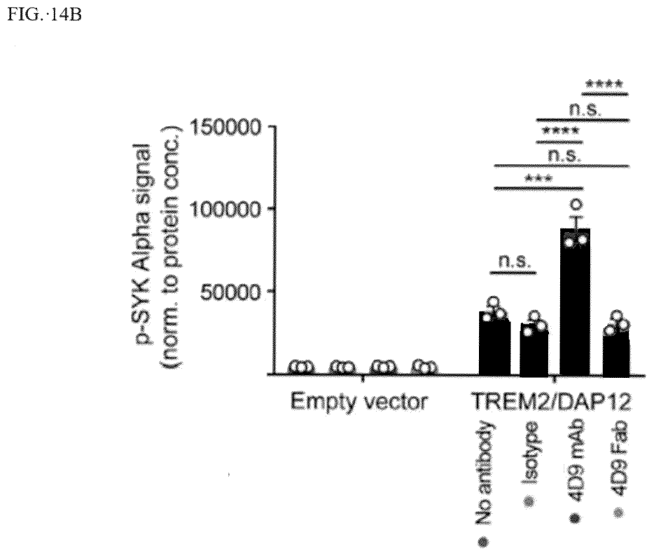

FIGS. 13A and 14A present data indicating that by inhibiting TREM2 cleavage, several of the T2GE antibodies (7F12, 13F4, 9D10 and 14B3) obtained by the screening assay described herein preserves and/or stimulates activity of microglia cells, for example Syk activation. It is known in the art that upon ligand binding to TREM2, tyrosine residues within ITAM are phosphorylated, recruiting Syk kinase to activate downstream signaling molecules such as extracellular signal-regulated protein kinase (ERK), phosphatidylinositol 3-kinase (PI3K), phospholipase Cγ (PLCγ), and Vav (Takahashi et al., 2005; Otero et al., 2009; Peng et al., 2010; Wang et al., 2015; Colonna and Wang, 2016).

### Example 2

Antibodies Generated Against GESESFEDAHV (SEQ ID NO:2).

The following antibody clones were generated against a peptide having the amino acid sequence of GESESFEDAHV (SEQ ID NO:2) corresponding to residues 145-155 of human TREM2 and inhibit TREM2 cleavage: 13H3; 9D10; 7F12; 13F4; 14B3

An antibody based on antibody clone 9D10, wherein the heavy chain variable region comprises the sequence of SEQ ID NO: 4 and the light chain variable region comprises the sequence of SEQ ID NO: 5; and wherein the antibody inhibits TREM2 cleavage. An antibody, wherein the heavy chain variable region comprises a sequence having at least 60%, preferably at least 85%, preferably at least 90%, more preferably at least 95%, even more preferably at least 98%, and most preferably at least 99% identity to SEQ ID NO: 4, and the light chain variable region comprises a sequence having at least 60%, preferably at least 85%, preferably at least 90%, more preferably at least 95%, even more preferably at least 98%, and most preferably at least 99% identity to SEQ ID NO: 5; and wherein the antibody inhibits TREM2 cleavage.

An antibody comprising one or more or all of the following CDR: the CDR1 of the heavy chain variable region positions 23-35 of SEQ ID NO:4, i.e., AASGFTFSDYGMA; the CDR2 of the heavy chain variable region positions 50-59 of SEQ ID NO:4, i.e., SISNSGGRTF; the CDR3 of the heavy chain variable region (IGGGSTFDY; positions 99-107 of SEQ ID NO:4); the CDR1 of the light chain variable region positions 24-39 of SEQ ID NO:5, i.e., KSSQSLVYSDGKTYLH; the CDR2 of the light chain variable region, positions 54-61 of SEQ ID NO:5, i.e., YQVSNLGS, and the CDR3 of the light chain variable region, positions 94-102 of SEQ ID NO:5, i.e., AQTTHFPPT, and wherein the antibody inhibits TREM2 cleavage.

An antibody comprising one or more or all of the following antigen binding regions, the antigen binding region corresponding to the CDR1 of the heavy chain variable region positions 27-35 of SEQ ID NO:4, i.e. FTFSDYGMA, the antigen binding region corresponding to the CDR2 of the heavy chain variable region positions 47-61 of SEQ ID NO:4, i.e., WVASISNSGGRTFYR, the antigen binding region corresponding to the CDR3 of the heavy chain variable region positions 97-107 of SEQ ID NO:4, i.e., TTIGGGSTFDY; the CDR1 of the light chain variable region positions 27-39 of SEQ ID NO:5, i.e., QSLVYSDGKTYLH; the antigen binding region corresponding to the CDR2 of the light chain variable region, positions 51-61 of SEQ ID NO:5, i.e., RLIYQVSNLGS and the antigen binding region corresponding to the CDR3 of the light chain variable region, positions 94-101 of SEQ ID NO:5, i.e., AQTTHFPP, and wherein the antibody inhibits TREM2 cleavage.

Clone 9D10 VH
SEQ ID NO 4
EVQLVESGGGLVQPGRSLKLSCAASGFTFSDYGMAWVRQAPTKGL
EWVASISNSGGRTFYRDSVKGRFTVSRDNAKSTLYLQMDSLRSEG
TATYYCTTIGGGSTEDYWGQGVMVTVSS
Clone 9D10 VL
SEQ ID NO 5
DVVMTQTPPSLSVAIGQSVSISCKSSQSLVYSDGKTYLHWLLQSP
GRSPKRLIYQVSNLGSGVPDRFSGTGSQKDFTLKISRVEAEDLGV
YYCAQTTHFPPTFGSGTKLEIK

An antibody based on antibody clone 7F12, wherein the heavy chain variable region comprises the sequence of SEQ ID NO: 6 and the light chain variable region comprises the sequence of SEQ ID NO: 7; and wherein the antibody inhibits TREM2 cleavage. An antibody, wherein the heavy chain variable region comprises a sequence having at least 60%, preferably at least 85%, preferably at least 90%, more preferably at least 95%, even more preferably at least 98%, and most preferably at least 99% identity to SEQ ID NO: 6, and the light chain variable region comprises a sequence having at least 60%, preferably at least 85%, preferably at least 90%, more preferably at least 95%, even more preferably at least 98%, and most preferably at least 99% identity to SEQ ID NO: 7; and wherein the antibody inhibits TREM2 cleavage. An antibody comprising one or more or all of the following CD regions; the CDR1 of the heavy chain variable region (positions 33-45 of SEQ ID NO:6, i.e., AASGFTFSNYGMA), the CDR2 of the heavy chain variable region (positions 50-59 of SEQ ID NO:6, i.e., SISYSGGRTY), the CDR3 of the heavy chain variable region)(e.g., PVGGGSTFDY positions 98-107); the CDR1 of the light chain variable region (positions 24-39 of SEQ ID NO:7, i.e., KSSQSLVYSDGKTYLH), the CDR2 of the light chain variable region (positions 54-61 of SEQ ID NO:7, i.e., YQVSNLGS), and the CDR3 of the light chain variable region (positions 94-101 of SEQ ID NO:7, i.e., AQTTHFPP), and wherein the antibody inhibits TREM2 cleavage.

An antibody comprising one or more or all of the following antigen binding regions corresponding to the CD regions; the CDR1 of the heavy chain variable region (positions 27-35 of SEQ ID NO:6, i.e., FTFSNYGMA), the CDR2 of the heavy chain variable region (positions 47-61 of SEQ ID NO:6, i.e., WVTSISYSGGRTYYR), the CDR3 of the heavy chain variable region (positions 97-107 of SEQ ID NO:6, i.e., TPVGGGSTFDY), the CDR1 of the light chain variable region (positions 27-39 of SEQ ID NO:7, i.e., QSLVYSDGKTYLH), the CDR2 of the light chain variable region (positions 51-61 of SEQ ID NO:7, i.e., RLIYQVSNLGS), and the CDR3 of the light chain variable region (positions 94-101 of SEQ ID NO: 7, i.e., AQTTHFPP), and wherein the antibody inhibits TREM2 cleavage.

Clone 7F12 VH
SEQ ID NO 6
EVQLVESGGGLVQPGRSLKLSCAASGFTFSNYGMAWVRQAPTKGL
EWVTSISYSGGRTYYRDSVKGRFTISRDNAKSTLYLQMDSLRSED
SATYYCTPVGGGSTFDYWGQGVMVTVSS
Clone 7F12 VL
SEQ ID NO 7
DVVMTQTPPSLSVAIGQSVSISCKSSQSLVYSDGKTYLHWLLQSP
GRSPKRLIYQVSNLGSGVPDRFSGTGSQKDFTLKISRVEAEDLGV
YYCAQTTHFPPTFGSGTKLEIK

An antibody based on antibody clone 13F4, wherein the heavy chain variable region comprises the sequence of SEQ ID NO: 8 and the light chain variable region comprises the sequence of SEQ ID NO: 9 or SEQ ID NO: 87; and wherein the antibody inhibits TREM2 cleavage. An antibody, wherein the heavy chain variable region comprises a sequence having at least 60%, preferably at least 85%, preferably at least 90%, more preferably at least 95%, even more preferably at least 98%, and most preferably at least 99% identity to SEQ ID NO: 8, and the light chain variable region comprises a sequence having at least 60%, preferably at least 85%, preferably at least 90%, more preferably at least 95%, even more preferably at least 98%, and most preferably at least 99% identity to SEQ ID NO: 9 or SEQ ID NO: 87; and wherein the antibody inhibits TREM2 cleavage. An antibody comprising one or more or all of the following CD regions, the CDR1 of the heavy chain variable region (positions 23-35 of SEQ ID NO:8, i.e., AASRFTFSNYGMA), the CDR2 of the heavy chain variable region (positions 50-59 of SEQ ID NO:8, i.e., SISNSGGRTF), the CDR3 of the heavy chain variable region (TIG GGSTFDY, positions 98-107 or (TTIGGGSTFDY, 97-107 of SEQ ID NO:8); the CDR1 of the light chain variable region (positions 24-39 of SEQ ID NO:9 or SEQ ID NO: 87, i.e., KSSQSLVYSDGKTYLH), the CDR2 of the light chain variable region (positions 54-61 of SEQ ID NO:9 or SEQ ID NO: 87, i.e., YQVSNLGS), and the CDR3 of the light chain variable region (positions 94-102 of SEQ ID NO:9 or SEQ ID NO: 87, i.e., AQTTHFPPT), and wherein the antibody inhibits TREM2 cleavage.

An antibody comprising one or more or all of the following antigen binding sites correlating to a CDR, the CDR1 of the heavy chain variable region (positions 26-35 of SEQ ID NO:8, i.e., RFTFSNYGMA), the CDR2 of the heavy chain variable region (positions 47-61 of SEQ ID NO:8, i.e., WVASISNSGGRTFYR), the CDR3 of the heavy chain variable region (positions 98-107 of SEQ ID NO:8, i.e., TIGGGSTFDY), the CDR1 of the light chain variable region (positions 27-39 of SEQ ID NO:9 or SEQ ID NO:87, i.e., QSLVYSDGKTYLH), the CDR2 of the light chain variable region(positions 51-61 of SEQ ID NO:9 or SEQ ID NO:87 i.e., RLIYQVSNLGS), and the CDR3 of the light chain variable region(positions 94-101 of SEQ ID NO:9 or SEQ ID NO:87 i.e., AQTTHFPP), and wherein the antibody inhibits TREM2 cleavage.

Clone 13F4 VH
SEQ ID NO 8
EVHLVESGGGLVQPGRSLKLSCAASRFTFSNYGMAWVRQAPTKGL
EWVASISNSGGRTFYRDSVKARFTISRDSAESTLYLQMDSLRSED
TATYYCTTIGGGSTFDYWGQGVMVTVSS
Clone 13F4 VL
SEQ ID NO 9
DVVMTQTPPSLSVAIGQSVSISCKSSQSLVYSDGKTYLHWLLQSP
GRSPKRLIYQVSNLGSGVPDRFSGTGSQKDFTLKISRVEAEDLGV
YYCAQTTHEPPTFGSGTKLEIK
Clone 13F4 VL
SEQ ID NO 87
DVVMTQTPPSLSVAIGQSVSISCKSSQSLVYSDGKTYLHWLLQSP
GRSPKRLIYQVSNLGSGVPDRFSGTGLQKDFTLKISRVEAEDLGV
YYCAQTTHEPPTFGSGTKLEIK

An antibody based on antibody clone 14B3, wherein the heavy chain variable region comprises the sequence of SEQ ID NO: 10 and the light chain variable region comprises the sequence of SEQ ID NO: 11 or SEQ ID NO: 12 or SEQ ID NO:13 or SEQ ID NO:14; and wherein the antibody inhibits TREM2 cleavage. An antibody, wherein the heavy chain variable region comprises a sequence having at least 60%, preferably at least 85%, preferably at least 90%, more preferably at least 95%, even more preferably at least 98%, and most preferably at least 99% identity to SEQ ID NO: 10, and the light chain variable region comprises a sequence having at least 60%, preferably at least 85%, preferably at least 90%, more preferably at least 95%, even more preferably at least 98%, and most preferably at least 99% identity to SEQ ID NO: 11 or SEQ ID NO:12 or SEQ ID NO:13 or SEQ ID NO:14; and wherein the antibody inhibits TREM2 cleavage. An antibody comprising one or more or all of a CDR as the CDR1 of the heavy chain variable region (positions 23-35 of SEQ ID NO:10, i.e., AASGFSFSNYGMA), the CDR2 of the heavy chain variable region (positions 50-59 of SEQ ID NO: 10, i.e., SISNNGGRTF); the CDR3 of the heavy chain variable region TIGGGSTFDY or IGGGSTFDY positions 98-107 or 99-107, respectively of SEQ ID NO:10; the CDR1 of the light chain variable region (positions 24-39 of SEQ ID NO:11, i.e., KSSQSLVYSDGKTYLH), the CDR2 of the light chain variable region positions 54-61 of SEQ ID NO:11, i.e., YQVSNLGS), and the CDR3 of the light chain variable region (positions 94-102 of SEQ ID NO: 11, i.e., AQTTHFPPT); and wherein the antibody inhibits TREM2 cleavage.

An antibody comprising one or more or all of the following antibody binding region corresponding to a CDR, including the CDR1 of the heavy chain variable region (positions 27-35 of SEQ ID NO: 10, i.e., FSFSNYGMA), the CDR2 of the heavy chain variable region(positions 47-61 of SEQ ID NO: 10, i.e., WVASISNNGGRTFYR); the CDR3 of the heavy chain variable region(positions 97-107 of SEQ ID NO:10, i.e., TTIGGGSTFDY); the CDR1 of the light chain variable region(positions 27-39 of SEQ ID NO:11, i.e., QSLVYSDGKTYLH), the CDR2 of the light chain variable region(positions 51-61 of SEQ ID NO:11, i.e., RLIYQVSNLGS), and the CDR3 of the light chain variable region (positions 94-101 of SEQ ID NO: 11, i.e., AQTTHFPP); and wherein the antibody inhibits TREM2 cleavage.

Or an antibody comprising one or more or all of a CDR as the CDR1 of the heavy chain variable region (positions 23-35 of SEQ ID NO:10, i.e., AASGFSFSNYGMA), the CDR2 of the heavy chain variable region (positions 50-59 of SEQ ID NO: 10, i.e., SISNNGGRTF); the CDR3 of the heavy chain variable region (TIGGGSTFDY or IGGGSTFDY positions 98-107 or 99-107, respectively of SEQ ID NO:10); the CDR1 of the light chain variable region (positions 24-39 of SEQ ID NO:12, i.e., KSSRSLVYSDGKTYLY), the CDR2 of the light chain variable region (positions 54-61 of SEQ ID NO:12, i.e., YQVSNLGS), and the CDR3 of the light chain variable region (positions 94-102 of SEQ ID NO: 12, i.e., AQTTHFPPT); and wherein the antibody inhibits TREM2 cleavage.

An antibody comprising one or more or all of the following antibody binding region corresponding to a CDR, including the CDR1 of the heavy chain variable region (positions 27-35 of SEQ ID NO: 10, i.e., FSFSNYGMA), the CDR2 of the heavy chain variable region(positions 47-61 of SEQ ID NO: 10, i.e., WVASISNNGGRTFYR); the CDR3 of the heavy chain variable region (positions 97-107 of SEQ ID NO:10, i.e., TTIGGGSTFDY); the CDR1 of the light chain variable region (positions 27-39 of SEQ ID NO: 12, i.e., RSLVYSDGKTYLY), the CDR2 of the light chain variable region (positions 51-61 of SEQ ID NO:12, i.e., RLIYQVSNLGS), and the CDR3 of the light chain variable region (positions 94-101 of SEQ ID NO:12, i.e., AQTTHFPP); and wherein the antibody inhibits TREM2 cleavage.

Or an antibody comprising one or more or all of a CDR as the CDR1 of the heavy chain variable region (positions 23-35 of SEQ ID NO:10, i.e., AASGFSFSNYGMA), the CDR2 of the heavy chain variable region (positions 50-59 of SEQ ID NO:10, i.e., SISNNGGRTF); the CDR3 of the heavy chain variable region (TIGGGSTFDY or IGGGSTFDY positions 98-107 or 99-107, respectively of SEQ ID NO:10); the CDR1 of the light chain variable region (positions 24-39 of SEQ ID NO:13, i.e., KSSQSLVYSDGKTYLH), the CDR2 of the light chain variable region (positions 54-61 of SEQ ID NO:13, i.e., RLIYQVSNLGS), and the CDR3 of the light chain variable region (positions 94-102 of SEQ ID NO:13, i.e., AQTTHFPPT); and wherein the antibody inhibits TREM2 cleavage.

Or an antibody comprising one or more or all of the following antibody binding region corresponding to a CDR, including the CDR1 of the heavy chain variable region (positions 27-35 of SEQ ID NO:10, i.e., FSFSNYGMA), the CDR2 of the heavy chain variable region(positions 47-61 of SEQ ID NO:10, i.e., WVASISNNGGRTFYR); the CDR3 of the heavy chain variable region(positions 97-107 of SEQ ID NO:10, i.e., TTIGGGSTFDY); the CDR1 of the light chain variable region (positions 27-39 of SEQ ID NO:13, i.e., QSLVYSDGKTYLH), the CDR2 of the light chain variable region (positions 51-61 of SEQ ID NO: 13, i.e., RLIYQVSNLGS), and the CDR3 of the light chain variable region (positions 94-101 of SEQ ID NO:13, i.e., AQTTHFPP); and wherein the antibody inhibits TREM2 cleavage.

Or an antibody comprising one or more or all of a CDR as the CDR1 of the heavy chain variable region (positions 23-35 of SEQ ID NO:10, i.e., AASGFSFSNYGMA), the CDR2 of the heavy chain variable region (positions 50-59 of SEQ ID NO:10, i.e., SISNNGGRTF); the CDR3 of the heavy chain variable region (TIGGGSTFDY or IGGGSTFDY positions 98-107 or 99-107, respectively of SEQ ID NO:10); the CDR1 of the light chain variable region (positions 24-39 of SEQ ID NO:14, i.e., KSSQSLVYSDGKTYLH), the CDR2 of the light chain variable region (positions 54-61 of SEQ ID NO:14, i.e., YQVSNLGS), and the CDR3 of the light chain variable region (positions 94-102 of SEQ ID NO: 14, i.e., AQTTHFPPT); and wherein the antibody inhibits TREM2 cleavage.

Or an antibody comprising one or more or all of the following antibody binding region corresponding to a CDR, including the CDR1 of the heavy chain variable region(positions 27-35 of SEQ ID NO: 10, i.e., FSFSNYGMA), the CDR2 of the heavy chain variable region(positions 47-61 of SEQ ID NO:10, i.e., WVASISNNGGRTFYR); the CDR3 of the heavy chain variable region(positions 97-107 of SEQ ID NO:10, i.e., TTIGGGSTFDY); the CDR1 of the light chain variable region (positions 27-39 of SEQ ID NO:14, i.e., QSLVYSDGKTYLH), the CDR2 of the light chain variable region (positions 50-61 of SEQ ID NO:14, i.e., NRLIYQVSNLGS), and the CDR3 of the light chain variable region (positions 94-101 of SEQ ID NO: 14, i.e., AQTTHFPP); and wherein the antibody inhibits TREM2 cleavage.

Clone 14B3 VH
SEQ ID NO 10
EVQLVESGGGLVQPGRSLKLSCAASGFSFSNYGMAWVRQAPTKGL
EWVASISNNGGRTFYRDSVKGRFTISRDNAKSTLYLQMHSLRSED
TATYYCTTIGGGSTFDYWGQGVMVTVSS
Clone 14B3 VL
SEQ ID NO 11
DVVMTQTPPSLSVAIGQSVSMSCKSSQSLVYSDGKTYLHWLLQSP
GGSPKRLIYQVSNLGSGVPDRFSGTGSQKDFTLKISRVEAEDLGV
YYCAQTTHFPPTFGSGTKLEIK
Clone 14B3 VL
SEQ ID NO 12
DVVMTQTPPSLSVAIGQSVSMSCKSSRSLVYSDGKTYLYWLLQSP
GRSPKRLIYQVSNLGSGVPDRFSGTGSQKDFTLKISRVEAEDLGV
YYCAQTTHFPPTFGSGTKLEIK
Clone 14B3 VL
SEQ ID NO 13
DVVMTQTPPSLSVAIGQSVSMSCKSSQSLVYSDGKTYLHWLLQSP
GRSPKRLIYQVSNLGSGVPDRFSGTGSQKDFTLKISRVEAEDLGV
YYCAQTTHFPPTFGSGTKLEIK
Clone 14B3 VL
SEQ ID NO 14
DVVMTHTPPSLSVAIGQSISMSCKSSQSLVYSDGKTYLHWLLQSPG
RSPNRLIYQVSNLGSGVPDRFSGTGSQKDFTLKIXRVKAEDLGVY
YCAQTTHFPPTFGSGTKLEIK

Antibodies Generated Against AHVEHSISRS (SEQ ID NO:3).

The following antibody clones were generated against a peptide having the amino acid sequence of AHVEHSISRS (SEQ ID NO:3) corresponding to residues 51-62 of human TREM2 and inhibit TREM2 cleavage: 14D3, 14D8, 7A12, 8A11, 21A3, 10C3, 18F9, 15C5, 1G6.

An antibody based on antibody clone 14D3, wherein the heavy chain variable region comprises the sequence of SEQ ID NO: 15 and the light chain variable region comprises the sequence of SEQ ID NO:16; and wherein the antibody inhibits TREM2 cleavage. An antibody, wherein the heavy chain variable region comprises a sequence having at least 60%, preferably at least 85%, preferably at least 90%, more preferably at least 95%, even more preferably at least 98%, and most preferably at least 99% identity to SEQ ID NO: 15, and the light chain variable region comprises a sequence having at least 60%, preferably at least 85%, preferably at least 90%, more preferably at least 95%, even more preferably at least 98%, and most preferably at least 99% identity to SEQ ID NO: 16; and wherein the antibody inhibits TREM2 cleavage. An antibody, wherein the CDR1 of the heavy chain variable region comprises the amino acid sequence of SEQ ID NO: 17; the CDR2 of the heavy chain variable region comprises the amino acid sequence of SEQ ID NO: 18; the CDR3 of the heavy chain variable region comprises the amino acid sequence of SEQ ID NO: 19; the CDR1 of the light chain variable region comprises the amino acid sequence of SEQ ID NO: 20; the CDR2 of the light chain variable region comprises the amino acid sequence of SEQ ID NO: 21; and the CDR3 of the light chain variable region comprises the amino acid sequence of SEQ ID NO: 22; and wherein the antibody inhibits TREM2 cleavage.

Clone 14D3 VH
SEQ ID NO 15
Glu Val Lys Leu Leu Glu Phe Gly Gly Gly Leu
Val Gln Pro Gly Gly Ser Met Arg Leu Ser Cys
Ala Ala Ser Gly Phe Thr Phe Thr Asp Phe Tyr
Met Asn Trp Ile Arg Gln Pro Ala Gly Arg Ala
Pro Glu Trp Leu Gly Leu Ile Arg Asn Lys Thr
Lys Gly Tyr Thr Thr Glu Tyr Asn Arg Ser Val
Lys Gly Arg Phe Thr Ile Ser Arg Asp Asn Thr
Gln Asn Met Leu Tyr Leu Gln Met Asn Ser Leu
Arg Pro Glu Asp Thr Ala Thr Tyr Tyr Cys Ala
Arg Ile Gly Val Asn Asn Gly Gly Ser Leu Asp
Tyr Trp Gly Gln Gly Val Met Val Thr Val Ser
Ser
Clone 14D3 VL
SEQ ID NO 16
Asp Ile Leu Ile Ile Gln Ser Pro Ala Ser Leu
Thr Val Ser Ala Gly Ala Arg Val Thr Met Ser
Cys Lys Ser Ser Gln Ser Leu Leu Tyr Ser Glu
Asn Asn Gln Asp Tyr Leu Ala Trp Tyr Gln Gln
Lys Pro Gly Gln Phe Pro Lys Leu Leu Ile Tyr
Gly Ala Ser Asn Arg His Thr Gly Val Pro Asp
Arg Phe Thr Gly Ser Gly Ser Gly Thr Asp Phe
Thr Leu Thr Ile Ser Ser Val Gln Ala Glu Asp
Leu Ala Asp Tyr Tyr Cys Glu Gln Thr Tyr Ser
Tyr Pro Tyr Thr Phe Gly Ala Gly Thr Lys Leu
Glu Leu Lys
<210>
Clone 14D3 CDR1 of the heavy chain
SEQ ID NO 17
Gly Phe Thr Phe Thr Asp Phe Tyr
Clone 14D3 CDR2 of the heavy chain
SEQ ID NO 18
Ile Arg Asn Lys Thr Lys Gly Tyr Thr Thr
Clone 14D3 CDR3 of the heavy chain
SEQ ID NO 19
Ala Arg Ile Gly Val Asn Asn Gly Gly Ser
Leu Asp Tyr Trp Gly
Clone 14D3 CDR1 of the light chain
SEQ ID NO 20
Gln Ser Leu Leu Tyr Ser Glu Asn Asn Gln
Asp Tyr
Clone 14D3 CDR2 of the light chain
SEQ ID NO 21
Gly Ala Ser
Clone 14D3 CDR3 of the light chain
SEQ ID NO 22
Glu Gln Thr Tyr Ser Tyr Pro Tyr Thr

An antibody based on antibody clone 14D8, wherein the heavy chain variable region comprises the sequence of SEQ ID NO: 23 and the light chain variable region comprises the sequence of SEQ ID NO: 24; and wherein the antibody inhibits TREM2 cleavage. An antibody, wherein the heavy chain variable region comprises a sequence having at least 60%, preferably at least 85%, preferably at least 90%, more preferably at least 95%, even more preferably at least 98%, and most preferably at least 99% identity to SEQ ID NO: 23, and the light chain variable region comprises a sequence having at least 60%, preferably at least 85%, preferably at least 90%, more preferably at least 95%, even more preferably at least 98%, and most preferably at least 99% identity to SEQ ID NO: 24; and wherein the antibody inhibits TREM2 cleavage. An antibody, wherein the CDR1 of the heavy chain variable region comprises the amino acid sequence of SEQ ID NO: 25; the CDR2 of the heavy chain variable region comprises the amino acid sequence of SEQ ID NO: 26; the CDR3 of the heavy chain variable region comprises the amino acid sequence of SEQ ID NO: 27; the CDR1 of the light chain variable region comprises the amino acid sequence of SEQ ID NO: 28; the CDR2 of the light chain variable region comprises the amino acid sequence of SEQ ID NO: 29; and the CDR3 of the light chain variable region comprises the amino acid sequence of SEQ ID NO: 30; and wherein the antibody inhibits

Clone 14D8 variable region of the heavy chain
SEQ ID NO 23
Glu Val Lys Leu Leu Glu Ser Gly Gly Gly Leu
Val Gln Pro Gly Gly Ser Met Arg Leu Ser Cys
Ala Ala Ser Gly Phe Thr Phe Thr Asp Phe Tyr
Met Asn Trp Ile Arg Gln Pro Ala Gly Lys Ala
Pro Glu Trp Leu Gly Leu Ile Arg Asn Lys Ala
Asn Gly Tyr Thr Thr Val Tyr Asn Pro Ser Val
Lys Gly Arg Phe Thr Ile Ser Arg Asp Asn Thr
Gln Asn Met Leu Tyr Leu Gln Met Asn Thr Leu
Arg Gly Glu Asp Thr Ala Thr Tyr Tyr Cys Ala
Arg Ile Gly Ile Asn Asn Gly Gly Ser Leu Asp
Tyr Trp Gly Gln Gly Val Met Val Thr Val Ser
Ser
Clone 14D8 variable region of the light chain
SEQ ID NO 24
Asp Ile Leu Ile Asn Gln Ser Pro Ala Ser Leu
Thr Val Ser Thr Gly Glu Lys Val Thr Met Ser
Cys Arg Ser Ser Gln Ser Leu Leu Tyr Ser Glu
Lys Asn Gln Asp Tyr Leu Ala Trp Tyr Gln Gln
Lys Pro Gly Gln Phe Pro Lys Leu Leu Ile Tyr
Gly Ala Ser Tyr Arg His Thr Gly Val Pro Asp
Arg Phe Thr Gly Ser Gly Ser Gly Thr Asp Phe
Thr Leu Thr Ile Ser Ser Val Gln Ala Glu Asp
Leu Ala Asp Tyr Tyr Cys Glu Gln Thr Tyr Ser
Tyr Pro Tyr Thr Phe Gly Ala Gly Thr Lys Leu
Glu Leu Lys
Clone 14D8 CDR1 of the heavy chain
SEQ ID NO 25
Gly Phe Thr Phe Thr Asp Phe Tyr
Clone 14D8 CDR2 of the heavy chain
SEQ ID NO 26
Ile Arg Asn Lys Ala Asn Gly Tyr Thr Thr
Clone 14D8 CDR3 of the heavy chain
SEQ ID NO 27
Ala Arg Ile Gly Ile Asn Asn Gly Gly Ser
Leu Asp Tyr Trp Gly
Clone 14D8 CDR1 of the light chain
SEQ ID NO 28
Gln Ser Leu Leu Tyr Ser Glu Lys Asn Gln Asp Tyr
Clone 14D8 CDR2 of the light chain
SEQ ID NO 29
Gly Ala Ser
Clone 14D8 CDR3 of the light chain
SEQ ID NO 30
Glu Gln Thr Tyr Ser Tyr Pro Tyr Thr

An antibody based on antibody clone 7A12, wherein the heavy chain variable region comprises the sequence of SEQ ID NO: 31 and the light chain variable region comprises the sequence of SEQ ID NO: 32; and wherein the antibody inhibits TREM2 cleavage. An antibody, wherein the heavy chain variable region comprises a sequence having at least 60%, preferably at least 85%, preferably at least 90%, more preferably at least 95%, even more preferably at least 98%, and most preferably at least 99% identity to SEQ ID NO: 315, and the light chain variable region comprises a sequence having at least 60%, preferably at least 85%, preferably at least 90%, more preferably at least 95%, even more preferably at least 98%, and most preferably at least 99% identity to SEQ ID NO: 32; and wherein the antibody inhibits TREM2 cleavage. An antibody, wherein the CDR1 of the heavy chain variable region comprises the amino acid sequence of SEQ ID NO: 33; the CDR2 of the heavy chain variable region comprises the amino acid sequence of SEQ ID NO: 34; the CDR3 of the heavy chain variable region comprises the amino acid sequence of SEQ ID NO: 35; the CDR1 of the light chain variable region comprises the amino acid sequence of SEQ ID NO: 36; the CDR2 of the light chain variable region comprises the amino acid sequence of SEQ ID NO: 37; and the CDR3 of the light chain variable region comprises the amino acid sequence of SEQ ID NO: 38; and wherein the antibody inhibits

Clone 7A12 variable region of the heavy chain
SEQ ID NO 31
Glu Val Lys Leu Leu Glu Ser Gly Gly Gly Leu
Val Gln Pro Gly Gly Ser Met Arg Leu Ser Cys
Ala Ala Ser Gly Phe Thr Phe Thr Asp Phe Tyr
Met Asn Trp Ile Arg Gln Pro Ala Gly Lys Ala
Pro Glu Trp Leu Gly Leu Ile Arg Asn Lys Ala
Asn Gly Tyr Thr Thr Gln Tyr Asn Pro Ser Val
Lys Gly Arg Phe Thr Ile Ser Arg Asp Asn Thr
Gln Asn Met Leu Tyr Leu Gln Met Asn Thr Leu
Arg Gly Glu Asp Thr Ala Thr Tyr Tyr Cys Ala
Arg Ile Gly Ile Asn Asn Gly Gly Ser Leu Asp
Tyr Trp Gly Gln Gly Val Met Val Thr Val Ser
Ser
Clone 7A12 variable region of the light chain
SEQ ID NO 32
Asp Ile Leu Ile Asn Gln Ser Pro Ala Ser Leu
Thr Val Ser Ala Gly Glu Lys Val Thr Met Ser
Cys Lys Ser Ser Gln Ser Leu Leu Tyr Ser Glu
Lys Asn Gln Asp Tyr Leu Ala Trp Tyr Gln Gln
Lys Pro Gly Gln Ser Pro Lys Leu Leu Met Tyr
Gly Ala Ser Tyr Arg His Thr Gly Val Pro Asp
Arg Phe Thr Gly Ser Gly Ser Gly Thr Asp Phe
Thr Leu Thr Ile Ser Ser Val Gln Ala Glu Asp
Leu Ala Asp Tyr Tyr Cys Glu Gln Thr Tyr Ser
Tyr Pro Tyr Thr Phe Gly Ala Gly Thr Lys Leu
Glu Leu Lys
Clone 7A12 CDR1 of the heavy chain
SEQ ID NO 33
Gly Phe Thr Phe Thr Asp Phe Tyr
Clone 7A12 CDR2 of the heavy chain
SEQ ID NO 34
Ile Arg Asn Lys Ala Asn Gly Tyr Thr Thr
Clone 7A12 CDR3 of the heavy chain
SEQ ID NO 35
Ala Arg Ile Gly Ile Asn Asn Gly Gly Ser
Leu Asp Tyr Trp Gly
Clone 7A12 CDR1 of the light chain
SEQ ID NO 36
Gln Ser Leu Leu Tyr Ser Glu Lys Asn Gln Asp Tyr
Clone 7A12 CDR2 of the light chain
SEQ ID NO 37
Gly Ala Ser
Clone 7A12 CDR3 of the light chain
SEQ ID NO 38
Glu Gln Thr Tyr Ser Tyr Pro Tyr Thr

An antibody based on antibody clone 8A11, wherein the heavy chain variable region comprises the sequence of SEQ ID NO: 39 and the light chain variable region comprises the sequence of SEQ ID NO: 40; and wherein the antibody inhibits TREM2 cleavage. An antibody, wherein the heavy chain variable region comprises a sequence having at least 60%, preferably at least 85%, preferably at least 90%, more preferably at least 95%, even more preferably at least 98%, and most preferably at least 99% identity to SEQ ID NO: 39 and the light chain variable region comprises a sequence having at least 60%, preferably at least 85%, preferably at least 90%, more preferably at least 95%, even more preferably at least 98%, and most preferably at least 99% identity to SEQ ID NO: 406; and wherein the antibody inhibits TREM2 cleavage. An antibody, wherein the CDR1 of the heavy chain variable region comprises the amino acid sequence of SEQ ID NO: 41; the CDR2 of the heavy chain variable region comprises the amino acid sequence of SEQ ID NO: 42; the CDR3 of the heavy chain variable region comprises the amino acid sequence of SEQ ID NO: 43; the CDR1 of the light chain variable region comprises the amino acid sequence of SEQ ID NO: 44; the CDR2 of the light chain variable region comprises the amino acid sequence of SEQ ID NO: 45; and the CDR3 of the light chain variable region comprises the amino acid sequence of SEQ ID NO: 46; and wherein the antibody inhibits

Clone 8A11 variable region of the heavy chain
SEQ ID NO 39
Glu Val Lys Leu Leu Glu Ser Gly Gly Gly Leu
Val Gln Pro Gly Gly Ser Met Arg Leu Ser Cys
Ala Ala Ser Gly Phe Thr Phe Thr Asp Phe Tyr
Met Asn Trp Ile Arg Gln Pro Ala Gly Lys Ala
Pro Glu Trp Leu Gly Leu Ile Arg Asn Lys Thr
Lys Gly Tyr Thr Thr Glu Tyr Asn Thr Ser Val
Lys Gly Arg Phe Thr Ile Ser Arg Asp Asn Thr
Gln Asn Met Leu Tyr Leu Gln Met Asn Ser Leu
Arg Pro Glu Asp Thr Ala Thr Tyr Tyr Cys Ala
Arg Ile Gly Val Asn Asn Gly Gly Ser Leu Asp
Tyr Trp Gly Gln Gly Val Met Val Thr Val Ser
Ser
Clone 8A11 variable region of the light chain
SEQ ID NO 40
Asp Ile Leu Ile Ile Gln Ser Pro Ala Ser Leu
Thr Val Ser Ala Gly Ala Arg Val Thr Met Ser
Cys Lys Ser Ser Gln Ser Leu Leu Tyr Ser Glu
Asn Asn Gln Asp Tyr Leu Ala Trp Tyr Gln Gln
Lys Pro Gly Gln Phe Pro Lys Leu Leu Ile Tyr
Gly Ala Ser Asn Arg His Thr Gly Val Pro Asp
Arg Phe Thr Gly Ser Gly Ser Gly Thr Asp Phe
Thr Leu Thr Ile Ser Ser Val Gln Ala Glu Asp
Leu Ala Asp Tyr Tyr Cys Glu Gln Thr Tyr Ser
Tyr Pro Tyr Thr Phe Gly Ala Gly Thr Lys Leu
Glu Leu
Clone 8A11 CDR1 of the heavy chain
SEQ ID NO 41
Gly Phe Thr Phe Thr Asp Phe Tyr
Clone 8A11 CDR2 of the heavy chain
SEQ ID NO 42
Ile Arg Asn Lys Thr Lys Gly Tyr Thr Thr
Clone 8A11 CDR3 of the heavy chain
SEQ ID NO 43
Ala Arg Ile Gly Val Asn Asn Gly Gly Ser
Leu Asp Tyr Trp Gly
Clone 8A11 CDR1 of the light chain
SEQ ID NO 44
Gln Ser Leu Leu Tyr Ser Glu Asn Asn Gln
Asp Tyr
Clone 8A11 CDR2 of the light chain
SEQ ID NO 45
Gly Ala Ser
Clone 8A11 CDR3 of the light chain
SEQ ID NO 46
Glu Gln Thr Tyr Ser Tyr Pro Tyr Thr
Lys

An antibody based on antibody clone 21A3, wherein the heavy chain variable region comprises the sequence of SEQ ID NO: 47 and the light chain variable region comprises the sequence of SEQ ID NO: 48; and wherein the antibody inhibits TREM2 cleavage An antibody, wherein the heavy chain variable region comprises a sequence having at least 60%, preferably at least 85%, preferably at least 90%, more preferably at least 95%, even more preferably at least 98%, and most preferably at least 99% identity to SEQ ID NO: 47, and the light chain variable region comprises a sequence having at least 60%, preferably at least 85%, preferably at least 90%, more preferably at least 95%, even more preferably at least 98%, and most preferably at least 99% identity to SEQ ID NO: 48; and wherein the antibody inhibits TREM2 cleavage. An antibody, wherein the CDR1 of the heavy chain variable region comprises the amino acid sequence of SEQ ID NO: 49; the CDR2 of the heavy chain variable region comprises the amino acid sequence of SEQ ID NO: 50; the CDR3 of the heavy chain variable region comprises the amino acid sequence of SEQ ID NO: 51; the CDR1 of the light chain variable region comprises the amino acid sequence of SEQ ID NO: 52; the CDR2 of the light chain variable region comprises the amino acid sequence of SEQ ID NO: 53; and the CDR3 of the light chain variable region comprises the amino acid sequence of SEQ ID NO: 54; and wherein the antibody inhibits

Clone 21A3 variable region of the heavy chain
SEQ ID NO 47
Glu Val Lys Leu Leu Glu Ser Gly Gly Gly Leu
Val Gln Pro Gly Gly Ser Met Arg Leu Ser Cys
Ala Ala Ser Gly Phe Thr Phe Thr Asp Phe Tyr
Met Asn Trp Ile Arg Gln Pro Ala Gly Lys Ala
Pro Glu Trp Leu Gly Leu Ile Arg Asn Lys Ala
Asn Gly Tyr Thr Thr Gln Tyr Asn Pro Ser Val
Lys Gly Arg Phe Thr Ile Ser Arg Asp Asn Thr
Gln Asn Met Leu Tyr Leu Gln Met Asn Thr Leu
Arg Gly Glu Asp Thr Ala Thr Tyr Tyr Cys Ala
Arg Ile Gly Ile Asn Asn Gly Gly Ser Leu Asp
Tyr Trp Gly Gln Gly Val Met Val Thr Val Ser
Ser
Clone 21A3 variable region of the light chain
SEQ ID NO 48
Asp Ile Leu Ile Asn Gln Ser Pro Ala Ser Leu
Thr Val Ser Ala Gly Glu Lys Val Thr Met Ser
Cys Lys Ser Ser Gln Ser Leu Leu Tyr Ser Glu
Lys Asn Gln Asp Tyr Leu Ala Trp Tyr Gln Gln
Lys Pro Gly Gln Ser Pro Lys Leu Leu Met Tyr
Gly Ala Ser Tyr Arg His Thr Gly Val Pro Asp
Arg Phe Thr Gly Ser Gly Ser Gly Thr Asp Phe
Thr Leu Thr Ile Ser Ser Val Gln Ala Glu Asp
Leu Ala Asp Tyr Tyr Cys Glu Gln Thr Tyr Ser
Tyr Pro Tyr Thr Phe Gly Ala Gly Thr Lys Leu
Glu Leu Lys
Clone 21A3 CDR1 of the heavy chain
SEQ ID NO 49
Gly Phe Thr Phe Thr Asp Phe Tyr
Clone 21A3 CDR2 of the heavy chain
SEQ ID NO 50
Ile Arg Asn Lys Ala Asn Gly Tyr Thr Thr
Clone 21A3 CDR3 of the heavy chain
SEQ ID NO 51
Ala Arg Ile Gly Ile Asn Asn Gly Gly Ser
Leu Asp Tyr Trp Gly
Clone 21A3 CDR1 of the light chain
SEQ ID NO 52
Gln Ser Leu Leu Tyr Ser Glu Lys Asn Gln
Asp Tyr
Clone 21A3 CDR2 of the light chain
SEQ ID NO 53
Gly Ala Ser
Clone 21A3 CDR3 of the light chain
SEQ ID NO 54
Glu Gln Thr Tyr Ser Tyr Pro Tyr Thr

An antibody based on antibody clone 10C3, wherein the heavy chain variable region comprises the sequence of SEQ ID NO: 55 and the light chain variable region comprises the sequence of SEQ ID NO: 56; and wherein the antibody inhibits TREM2 cleavage. An antibody, wherein the heavy chain variable region comprises a sequence having at least 60%, preferably at least 85%, preferably at least 90%, more preferably at least 95%, even more preferably at least 98%, and most preferably at least 99% identity to SEQ ID NO: 55, and the light chain variable region comprises a sequence having at least 60%, preferably at least 85%, preferably at least 90%, more preferably at least 95%, even more preferably at least 98%, and most preferably at least 99% identity to SEQ ID NO: 56; and wherein the antibody inhibits TREM2 cleavage. An antibody, wherein the CDR1 of the heavy chain variable region comprises the amino acid sequence of SEQ ID NO: 57; the CDR2 of the heavy chain variable region comprises the amino acid sequence of SEQ ID NO: 58; the CDR3 of the heavy chain variable region comprises the amino acid sequence of SEQ ID NO: 59; the CDR1 of the light chain variable region comprises the amino acid sequence of SEQ ID NO: 60; the CDR2 of the light chain variable region comprises the amino acid sequence of SEQ ID NO: 61; and the CDR3 of the light chain variable region comprises the amino acid sequence of SEQ ID NO: 62; and wherein the antibody inhibits TREM2 cleavage.

Clone 10C3 variable region of the heavy chain
SEQ ID NO 55
Glu Val Lys Leu Leu Glu Ser Gly Gly Gly Leu
Val Gln Pro Gly Gly Ser Met Arg Leu Ser Cys
Ala Ala Ser Gly Phe Thr Phe Thr Asp Phe Tyr
Met Asn Trp Ile Arg Gln Pro Ala Gly Glu Thr
Pro Glu Trp Leu Gly Leu Ile ArgAsn Lys Thr
Lys Gly Tyr Thr Thr Glu Tyr Asn Pro Ser Val
Lys Gly Arg Phe Thr Ile Ser Arg Asp Asn Thr
Gln Asn Met Leu Tyr Leu Gln Met Asn Ser Leu
Arg Pro Glu Asp Thr Ala Thr Tyr Tyr Cys Ala
Arg Ile Gly Thr Asn Asn Gly Gly Ser Leu Asp
Tyr Trp Gly Gln Gly Val Met Val Thr Val Ser
Ser
Clone 10C3 variable region of the light chain
SEQ ID NO 56
Asp Ile Leu Ile Ile Gln Ser Pro Ala Ser Leu
Thr Val Ser Ala Gly Ala Arg Val Thr Met Ser
Cys Lys Ser Ser Gln Ser Leu Leu Tyr Ser Glu
Asn Asn Gln Asp Tyr Leu Ala Trp Tyr Gln Gln
Lys Pro Gly Gln Phe Pro Lys Leu Leu Ile Tyr
Gly Ala Ser Asn Arg His Thr Gly Val Pro Asp
Arg Phe Thr Gly Ser Gly Ser Gly Thr Asp Phe
Thr Leu Thr Ile Ser Ser Val Gln Ala Glu Asp
Leu Ala Asp Tyr Tyr Cys Glu Gln Thr Tyr Ser
Tyr Pro Tyr Thr Phe Gly Ala Gly Thr Lys Leu
Glu Leu Lys
Clone 10C3 CDR1 of the heavy chain
SEQ ID NO 57
Gly Phe Thr Phe Thr Asp Phe Tyr
Clone 10C3 CDR2 of the heavy chain
SEQ ID NO 58
Ile Arg Asn Lys Thr Lys Gly Tyr Thr Thr
Clone 10C3 CDR3 of the heavy chain
SEQ ID NO 59
Ala Arg Ile Gly Thr Asn Asn Gly Gly Ser
Leu Asp Tyr Trp Gly
Clone 10C3 CDR1 of the light chain
SEQ ID NO 60
Gln Ser Leu Leu Tyr Ser Glu Asn Asn Gln Asp Tyr
Clone 10C3 CDR2 of the light chain
SEQ ID NO 61
Gly Ala Ser
Clone 10C3 CDR3 of the light chain
SEQ ID NO 62
Glu Gln Thr Tyr Ser Tyr Pro Tyr Thr

An antibody based on antibody clone 18F9, wherein the heavy chain variable region comprises the sequence of SEQ ID NO: 63 and the light chain variable region comprises the sequence of SEQ ID NO: 64; and wherein the antibody inhibits TREM2 cleavage. An antibody, wherein the heavy chain variable region comprises a sequence having at least 60%, preferably at least 85%, preferably at least 90%, more preferably at least 95%, even more preferably at least 98%, and most preferably at least 99% identity to SEQ ID NO: 63, and the light chain variable region comprises a sequence having at least 60%, preferably at least 85%, preferably at least 90%, more preferably at least 95%, even more preferably at least 98%, and most preferably at least 99% identity to SEQ ID NO: 64; and wherein the antibody inhibits TREM2 cleavage. An antibody, wherein the CDR1 of the heavy chain variable region comprises the amino acid sequence of SEQ ID NO: 65; the CDR2 of the heavy chain variable region comprises the amino acid sequence of SEQ ID NO: 66; the CDR3 of the heavy chain variable region comprises the amino acid sequence of SEQ ID NO: 67; the CDR1 of the light chain variable region comprises the amino acid sequence of SEQ ID NO: 68; the CDR2 of the light chain variable region comprises the amino acid sequence of SEQ ID NO: 69; and the CDR3 of the light chain variable region comprises the amino acid sequence of SEQ ID NO: 70; and wherein the antibody inhibits

Clone 18F9 variable region of the heavy chain
SEQ ID NO 63
Glu Val Lys Leu Leu Glu Ser Gly Gly Gly Leu
Val Gln Pro Gly Gly Ser Met Arg Leu Ser Cys
Val Val Ser Gly Phe Thr Phe Thr Asp Phe Tyr
Met Asn Trp Ile Arg Gln Ala Ala Gly Lys Ala
Pro Glu Trp Leu Gly Leu Ile Arg Asn Lys Val
Asn Gly Tyr Arg Thr Glu Tyr Asn Pro Ser Val
Lys Gly Arg Phe Thr Ile Ser Arg Asp Asn Ile
Gln Asn Met Leu Tyr Leu Gln Met Asn Thr Leu
Arg Ala Glu Asp Thr Ala Thr Tyr Tyr Cys Ala
Arg Ile Gly Ile Asn Asn Gly Gly Ser Leu Asp
Tyr Trp Gly Gln Gly Val Met Val Thr Val Ser
Ser
Clone 18F9 variable chain of the light region
SEQ ID NO 64
Asp Ile Leu Ile Asn Gln Ser Pro Ala Ser Leu
Thr Val Ser Ala Gly Glu Lys Val Thr Met Ser
Cys Lys Ser Ser Gln Ser Leu Leu Tyr Ser Glu
Asn Asn Gln Asp Tyr Leu Ala Trp Tyr Gln Gln
Lys Pro Gly Gln Phe Pro Lys Leu Leu Ile Tyr
Gly Ala Ser Asn Arg His Thr Gly Val Pro Asp
Arg Phe Thr Gly Ser Gly Ser Gly Thr Asp Phe
Thr Leu Thr Ile Ser Ser Val Gln Ala Glu Asp
Leu Ala Asp Tyr Tyr Cys Glu Gln Thr Tyr Ser
Tyr Pro Tyr Thr Phe Gly Ala Gly Thr Lys Leu
Glu Leu Lys
Clone 18F9 CDR1 of the heavy chain
SEQ ID NO 65
Gly Phe Thr Phe Thr Asp Phe Tyr
Clone 18F9 CDR2 of the heavy chain
SEQ ID NO 66
Ile Arg Asn Lys Val Asn Gly Tyr Arg Thr
Clone 18F9 CDR3 of the heavy chain
SEQ ID NO 67
Ala Arg Ile Gly Ile Asn Asn Gly Gly Ser Leu
Asp Tyr Trp Gly
Clone 18F9 CDR1 of the light chain
SEQ ID NO 68
Gln Ser Leu Leu Tyr Ser Glu Asn Asn Gln Asp
Tyr
Clone 18F9 CDR2 of the light chain
SEQ ID NO 69
Gly Ala Ser
Clone 18F9 CDR3 of the light chain
SEQ ID NO 70
Glu Gln Thr Tyr Ser Tyr Pro Tyr Thr

An antibody based on antibody clone 15C5, wherein the heavy chain variable region comprises the sequence of SEQ ID NO: 71 and the light chain variable region comprises the sequence of SEQ ID NO: 72; and wherein the antibody inhibits TREM2 cleavage. An antibody, wherein the heavy chain variable region comprises a sequence having at least 60%, preferably at least 85%, preferably at least 90%, more preferably at least 95%, even more preferably at least 98%, and most preferably at least 99% identity to SEQ ID NO: 71, and the light chain variable region comprises a sequence having at least 60%, preferably at least 85%, preferably at least 90%, more preferably at least 95%, even more preferably at least 98%, and most preferably at least 99% identity to SEQ ID NO: 72; and wherein the antibody inhibits TREM2 cleavage. An antibody, wherein the CDR1 of the heavy chain variable region comprises the amino acid sequence of SEQ ID NO: 73; the CDR2 of the heavy chain variable region comprises the amino acid sequence of SEQ ID NO: 74; the CDR3 of the heavy chain variable region comprises the amino acid sequence of SEQ ID NO: 75; the CDR1 of the light chain variable region comprises the amino acid sequence of SEQ ID NO: 76; the CDR2 of the light chain variable region comprises the amino acid sequence of SEQ ID NO: 77; and the CDR3 of the light chain variable region comprises the amino acid sequence of SEQ ID NO: 78; and wherein the antibody inhibits TREM2 cleavage.

Clone 15C5 variable region of the heavy chain
SEQ ID NO 71
Glu Val Lys Leu Leu Glu Ser Gly Gly Gly Leu
Val Gln Pro Gly Gly Ser Met Arg Leu Ser Cys
Ala Ala Ser Gly Phe Thr Phe Thr Asp Phe Tyr
Met Asn Trp Ile Arg Gln Pro Ala Gly Lys Ala
Pro Glu Trp Leu Gly Leu Ile Arg Asn Lys Ala
Tyr Gly Tyr Thr Thr Glu Tyr Asn Pro Ser Val
Lys Gly Arg Phe Thr Ile Ser Arg Asp Asn Thr
Gln Asp Met Leu Tyr Leu Gln Met Asn Thr Leu
Arg Ala Glu Asp Thr Ala Thr Tyr Tyr Cys Ala
Arg Ile Gly Ile Asn Tyr Gly Gly Ser Leu Asp
Tyr Trp Gly Gln Gly Val Met Val Thr Val Ser
Ser
Clone 15C5 variable region of the light chain
SEQ ID NO 72
Asp Ile Leu Ile Asn Gln Ser Pro Ala Ser Leu
Thr Val Ser Ala Gly Glu Lys Val Thr Val Ser
Cys Lys Ser Ser Gln Ser Leu Leu Tyr Ser Glu
Ser Asn Gln Asp Tyr Leu Ala Trp Tyr Gln Gln
Lys Pro Gly Gln Phe Pro Lys Leu Leu Ile Tyr
Gly Ala Ser Tyr Arg His Thr Gly Val Pro Asp
Arg Phe Thr Gly Ser Gly Ser Gly Thr Asp Phe
Thr Leu Thr Ile Ser Ser Val Gln Ala Glu Asp
Leu Ala His Tyr Tyr Cys Glu Gln Thr Tyr Ser
Tyr Pro Tyr Thr Phe Gly Ala Gly Thr Lys Leu
Glu Leu Lys
Clone 15C5 CDR1
SEQ ID NO 73
Gly Phe Thr Phe Thr Asp Phe Tyr
Clone 15C5 CDR2 of the heavy chain
SEQ ID NO 74
Ile Arg Asn Lys Ala Tyr Gly Tyr Thr Thr
Clone 15C5 CDR3 of the heavy chain
SEQ ID NO 75
Ala Arg Ile Gly Ile Asn Tyr Gly Gly Ser
Leu Asp Tyr Trp Gly
Clone 15C5 CDR1 of the light chain
SEQ ID NO 76
Gln Ser Leu Leu Tyr Ser Glu Ser Asn Gln
Asp Tyr
Clone 15C5 CDR2 of the light chain
SEQ ID NO 77
Gly Ala Ser
Clone 15C5 CDR3 of the light chain
SEQ ID NO 78
Glu Gln Thr Tyr Ser Tyr Pro Tyr Thr

An antibody based on antibody clone 1G6, wherein the heavy chain variable region comprises the sequence of SEQ ID NO: 79 and the light chain variable region comprises the sequence of SEQ ID NO: 80; and wherein the antibody inhibits TREM2 cleavage. An antibody, wherein the heavy chain variable region comprises a sequence having at least 60%, preferably at least 85%, preferably at least 90%, more preferably at least 95%, even more preferably at least 98%, and most preferably at least 99% identity to SEQ ID NO: 79, and the light chain variable region comprises a sequence having at least 60%, preferably at least 85%, preferably at least 90%, more preferably at least 95%, even more preferably at least 98%, and most preferably at least 99% identity to SEQ ID NO: 80; and wherein the antibody inhibits TREM2 cleavage. An antibody, wherein the CDR1 of the heavy chain variable region comprises the amino acid sequence of SEQ ID NO: 81; the CDR2 of the heavy chain variable region comprises the amino acid sequence of SEQ ID NO: 82; the CDR3 of the heavy chain variable region comprises the amino acid sequence of SEQ ID NO: 83; the CDR1 of the light chain variable region comprises the amino acid sequence of SEQ ID NO: 84; the CDR2 of the light chain variable region comprises the amino acid sequence of SEQ ID NO: 85; and the CDR3 of the light chain variable region comprises the amino acid sequence of SEQ ID NO: 86; and wherein the antibody inhibits

Clone 1G6 variable region of the heavy chain
SEQ ID NO 79
Glu Val Lys Leu Leu Glu Ser Gly Gly Gly Leu
Val Gln Pro Gly Gly Ser Leu Arg Leu Ser Cys
Val Ala Ser Gly Phe Thr Phe Thr Asp Phe Tyr
Met Asn Trp Ile Arg Gln Pro Ala Gly Lys Ala
Pro Glu Trp Leu Gly Leu Ile Arg Asn Lys Ala
Asn Gly Phe Thr Thr Glu Tyr Asn Pro Ser Val
Lys Gly Arg Phe Thr Ile Ser Arg Asp Asn Thr
Gln His Met Leu Tyr Leu Gln Met Asn Thr Leu
Arg Ala Glu Asp Thr Ala Thr Tyr Tyr Cys Ala
Arg Ile Gly Ile Asn Asn Gly Gly Ser Leu Asp
Tyr Trp Gly Gln Gly Val Met Val Thr Val Ser
Ser
Clone 1G6 variable region of the light chain
SEQ ID NO 80
Asp Ile Leu Ile Asn Gln Ser Pro Ala Ser Leu
Thr Val Ser Thr Gly Glu Lys Val Thr Met Ser
Cys Lys Ser Ser Gln Ser Leu Leu Tyr Ser Glu
Asn Lys Gln Asp Tyr Leu Ala Trp Tyr Gln Gln
Lys Pro Gly Gln Phe Pro Lys Leu Leu Ile Tyr
Gly Ala Ser Asn Arg His Thr Gly Val Pro Asp
Arg Phe Thr Gly Ser Gly Ser Gly Thr Asp Phe
Thr Leu Thr Ile Asn Ile Val Gln Ala Glu Asp
Leu Ala Asp Tyr Tyr Cys Glu Gln Thr Tyr Ser
Tyr Pro Tyr Thr Phe Gly Ala Gly Thr Lys Leu
Glu Leu
Clone 1G6 CDR1 of the heavy chain
SEQ ID NO 81
Gly Phe Thr Phe Thr Asp Phe Tyr
Clone 1G6 CDR2 of the heavy chain
SEQ ID NO 82
Ile Arg Asn Lys Ala Asn Gly Phe Thr Thr
Clone 1G6 CDR3 of the heavy chain
SEQ ID NO 83
Ala Arg Ile Gly Ile Asn Asn Gly Gly Ser Leu
Asp Tyr Trp Gly
Clone 1G6 CDR1 of the light chain
SEQ ID NO 84
Gln Ser Leu Leu Tyr Ser Glu Asn Lys Gln Asp Tyr
Clone 1G6 CDR2 of the light chain
SEQ ID NO 85
Gly Ala Ser
Clone 1G6 CDR3 of the light chain
SEQ ID NO 86
Glu Gln Thr Tyr Ser Tyr Pro Tyr Thr
Lys

### Example 3

A patient suffering from a disease as set forth herein, for example AMD, is treated as follows.

An effective amount of an antibody as described herein is delivered to the posterior of eye, as is described for any one of the known antibody drugs such as bevacizumab, ranibizumab, lapatinib, sunitinib, or sorafenib. If desired, the antibody is formulated such that the formulation contains cell penetrating peptides to help penetrate into the anterior segment of the eye, such as TAT (derived from transactivator of transcription of human immunodeficiency virus), CC12, PNT (Penetratin), or RGD (arginine-glycine-aspartic acid).

### Other Embodiments

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

Other embodiments are within the following claims.

1. A method for treating a degenerative and vascular disease of the retina and/or posterior segment of the eye in a subject, the method comprising administering to a subject an effective amount of an antibody or fragment thereof that binds to an epitope of GESESFEDAHV. (SEQ ID NO: 2).

2. A method for treating a degenerative and vascular disease of the retina and/or posterior segment of the eye in a subject, the method comprising administering to a subject an effective amount of an antibody or fragment thereof, wherein the antibody comprises a heavy chain variable region and a light chain variable region, wherein: (a) the heavy the heavy chain variable region comprises a sequence having at least 85% sequence identity to SEQ ID NO: 4 and wherein the heavy chain variable region comprises a CDR1 comprising positions 23-35 of SEQ ID NO:4, a CDR2 comprising positions 50-59 of SEQ ID NO:4, and a CDR3 comprising positions 99-107 of SEQ ID NO:4, and the light chain variable region comprises a sequence having at least 85% sequence identity to SEQ ID NO: 5 and wherein the light chain variable region comprises a CDR1 comprising positions 24-39 of SEQ ID NO:5, a CDR2 comprising positions 54-61 of SEQ ID NO:5, and a CDR3 comprising positions 94-102 of SEQ ID NO:5, and wherein the antibody inhibits TREM2 cleavage; or (b) the heavy the heavy chain variable region comprises a sequence having at least 85% sequence identity to SEQ ID NO: 6 and wherein the heavy chain variable region comprises a CDR1 comprising positions 33-45 of SEQ ID NO:6, a CDR2 comprising positions 50-59 of SEQ ID NO:6, and a CDR3 comprising positions 98-107 of SEQ ID NO:6, and the light chain variable region comprises a sequence having at least 85% sequence identity to SEQ ID NO:7 and wherein the light chain variable region comprises a CDR1 comprising positions 24-39 of SEQ ID NO:7, a CDR2 comprising positions 54-61 of SEQ ID NO:7, and a CDR3 comprising positions 94-101 of SEQ ID NO:7, and wherein the antibody inhibits TREM2 cleavage; or (c) the heavy the heavy chain variable region comprises a sequence having at least 85% sequence identity to SEQ ID NO: 8 and wherein the heavy chain variable region comprises a CDR1 comprising positions 23-35 of SEQ ID NO:8, a CDR2 comprising positions 50-59 of SEQ ID NO:8, and a CDR3 comprising positions 98-107 of SEQ ID NO:8, and the light chain variable region comprises a sequence having at least 85% sequence identity to SEQ ID NO:9 or to SEQ ID NO:87, wherein the light chain variable region comprises a CDR1 comprising positions 24-39 of SEQ ID NO:9, a CDR2 comprising positions 54-61 of SEQ ID NO:9, and a CDR3 comprising positions 94-102 of SEQ ID NO:9, or wherein the light chain variable region comprises a CDR1 comprising positions 24-39 of SEQ ID NO:87, a CDR2 comprising positions 54-61 of SEQ ID NO:87, and a CDR3 comprising positions 94-102 of SEQ ID NO:87, and wherein the antibody inhibits TREM2 cleavage; or (d) the heavy the heavy chain variable region comprises a sequence having at least 85% sequence identity to SEQ ID NO: 10 and wherein the heavy chain variable region comprises a CDR1 comprising positions 23-35 of SEQ ID NO:10, a CDR2 comprising positions 50-59 of SEQ ID NO:10, and a CDR3 comprising positions 99-107 of SEQ ID NO:10 or positions 98-107 of SEQ ID NO:10, and the light chain variable region comprises a sequence having at least 85% sequence identity to SEQ ID NO:11 or to SEQ ID NO:12 or to SEQ ID NO:13 or to SEQ ID NO:14, wherein the light chain variable region comprises a CDR1 comprising positions 24-39 of SEQ ID NO:11, a CDR2 comprising positions 54-61 of SEQ ID NO:11, and a CDR3 comprising positions 94-102 of SEQ ID NO:11, or wherein the light chain variable region comprises a CDR1 comprising positions 24-39 of SEQ ID NO:12, a CDR2 comprising positions 54-61 of SEQ ID NO:12, and a CDR3 comprising positions 94-102 of SEQ ID NO:12, or wherein the light chain variable region comprises a CDR1 comprising positions 24-39 of SEQ ID NO:13, a CDR2 comprising positions 54-61 of SEQ ID NO:13, and a CDR3 comprising positions 94-102 of SEQ ID NO:13, or wherein the light chain variable region comprises a CDR1 comprising positions 24-39 of SEQ ID NO:14, a CDR2 comprising positions 54-61 of SEQ ID NO:14, and a CDR3 comprising positions 94-102 of SEQ ID NO:14, and wherein the antibody inhibits TREM2 cleavage.

3. The method of claim 1 in which the eye disease comprises age-related macular degeneration.

4. The method according to claim 3 in which the eye disease comprises “dry” age-related macular degeneration.

5. The method of claim 1, in which the method further comprises administering to the subject at least one additional therapeutic agent.

6. The method according to claim 5, in which the at least one additional therapeutic agent comprises one or more microglia checkpoint inhibitors.

7. A method of screening for a molecule which specifically binds to and inhibits cleavage of human TREM2 between His 157 and/or Ser158 on the surface of retinal microglial cells by ADAM10 and/or ADAM17.

8. The method of claim 7, wherein said molecule is an antibody or fragment thereof.

9. The method of claim 7, wherein said method comprises immunizing a mammal with a peptide comprising the amino acid sequence GESESFEDAHV (SEQ ID NO:2) or AHVEHSISRS (SEQ ID NO:3).

10. The method of claim 7, wherein said method comprises determining the level of inhibition of cleavage of human TREM2 between His 157 and/or Ser158 by ADAM10 and/or ADAM17.

11. The method of claim 7, wherein said inhibition of cleavage of human TREM2 between His 157 and/or Ser158 by ADAM10 and/or ADAM17 occurs in human retinal microglial cells.

12. The method of claim 11, where the microglial cells are associated with neural cells.

13. The method of claim 12, wherein the neural cells are located in the brain.

14. The method of claim 12, wherein the neural cells are associated with the ocular tissue.

15. The method of claim 7, wherein said molecule increases the level of mature membrane-bound full-length TREM2 by shedding inhibition as evidenced by reduced levels of soluble TREM2 and/or the membrane retained C-terminal fragment (CTF).

16. The method of claim 7, wherein said molecule inhibits TREM2 shedding with IC50s in the low nM range.

17. The method of claim 7, wherein said molecule activates pSYK signaling.

18. The method of claim 7, wherein said molecule increases liposome mediated pSYK signaling.

19. The method of claim 2 in which the eye disease comprises age-related macular degeneration.

20. The method according to claim 19 in which the eye disease comprises “dry” age-related macular degeneration.

21. The method of claim 2, in which the method further comprises administering to the subject at least one additional therapeutic agent.

22. The method according to claim 21, in which the at least one additional therapeutic agent comprises one or more microglia checkpoint inhibitors.


SEQUENCE LISTING (87 sequences)

SEQ ID NO: 1 - protein, 30 aa, synthetic construct
  REGION 1..30: note = Amino acid sequence of Human TREM2, at positions 141-170
LWFPGESESFEDAHVEHSISRSLLEFEIPF

SEQ ID NO: 2 - protein, 11 aa, synthetic construct
  REGION 1..11: note = Peptide comprising the amino acid sequence of residues 145-155 of human TREM2
GESESFEDAHV

SEQ ID NO: 3 - protein, 10 aa, synthetic construct
  REGION 1..10: note = peptide comprising the amino acid sequence of residues 153-162 of human TREM2
AHVEHSISRS

SEQ ID NO: 4 - protein, 118 aa, synthetic construct
  REGION 1..118: note = Clone 9D10 VH
EVQLVESGGGLVQPGRSLKLSCAASGFTFSDYGMAWVRQAPTKGLEWVASISNSGGRTFYRDSVKGRFTVSRDNAKSTLYLQMDSLRSEGTATYYCTTIGGGSTFDYWGQGVMVTVSS

SEQ ID NO: 5 - protein, 112 aa, synthetic construct
  REGION 1..112: note = Clone 9D10 VL
DVVMTQTPPSLSVAIGQSVSISCKSSQSLVYSDGKTYLHWLLQSPGRSPKRLIYQVSNLGSGVPDRFSGTGSQKDFTLKISRVEAEDLGVYYCAQTTHFPPTFGSGTKLEIK

SEQ ID NO: 6 - protein, 118 aa, synthetic construct
  REGION 1..118: note = Clone 7F12 VH
EVQLVESGGGLVQPGRSLKLSCAASGFTFSNYGMAWVRQAPTKGLEWVTSISYSGGRTYYRDSVKGRFTISRDNAKSTLYLQMDSLRSEDSATYYCTPVGGGSTFDYWGQGVMVTVSS

SEQ ID NO: 7 - protein, 112 aa, synthetic construct; identical to SEQ ID NO: 5
  REGION 1..112: note = Clone 7F12 VL

SEQ ID NO: 8 - protein, 118 aa, synthetic construct
  REGION 1..118: note = Clone 13F4 VH
EVHLVESGGGLVQPGRSLKLSCAASRFTFSNYGMAWVRQAPTKGLEWVASISNSGGRTFYRDSVKARFTISRDSAESTLYLQMDSLRSEDTATYYCTTIGGGSTFDYWGQGVMVTVSS

SEQ ID NO: 9 - protein, 112 aa, synthetic construct; identical to SEQ ID NO: 5
  REGION 1..112: note = Clone 13F4 VL

SEQ ID NO: 10 - protein, 118 aa, synthetic construct
  REGION 1..118: note = Clone 14B3 VH
EVQLVESGGGLVQPGRSLKLSCAASGFSFSNYGMAWVRQAPTKGLEWVASISNNGGRTFYRDSVKGRFTISRDNAKSTLYLQMHSLRSEDTATYYCTTIGGGSTFDYWGQGVMVTVSS

SEQ ID NO: 11 - protein, 112 aa, synthetic construct
  REGION 1..112: note = Clone 14B3 VL
DVVMTQTPPSLSVAIGQSVSMSCKSSQSLVYSDGKTYLHWLLQSPGGSPKRLIYQVSNLGSGVPDRFSGTGSQKDFTLKISRVEAEDLGVYYCAQTTHFPPTFGSGTKLEIK

SEQ ID NO: 12 - protein, 112 aa, synthetic construct
  REGION 1..112: note = Clone 14B3 VL
DVVMTQTPPSLSVAIGQSVSMSCKSSRSLVYSDGKTYLYWLLQSPGRSPKRLIYQVSNLGSGVPDRFSGTGSQKDFTLKISRVEAEDLGVYYCAQTTHFPPTFGSGTKLEIK

SEQ ID NO: 13 - protein, 112 aa, synthetic construct
  REGION 1..112: note = Clone 14B3 VL
DVVMTQTPPSLSVAIGQSVSMSCKSSQSLVYSDGKTYLHWLLQSPGRSPKRLIYQVSNLGSGVPDRFSGTGSQKDFTLKISRVEAEDLGVYYCAQTTHFPPTFGSGTKLEIK

SEQ ID NO: 14 - protein, 112 aa, synthetic construct
  REGION 1..112: note = Clone 14B3 VL
DVVMTHTPPSLSVAIGQSISMSCKSSQSLVYSDGKTYLHWLLQSPGRSPNRLIYQVSNLGSGVPDRFSGTGSQKDFTLKIXRVKAEDLGVYYCAQTTHFPPTFGSGTKLEIK

SEQ ID NO: 15 - protein, 122 aa, synthetic construct
  REGION 1..122: note = Clone 14D3 VH
EVKLLEFGGGLVQPGGSMRLSCAASGFTFTDFYMNWIRQPAGRAPEWLGLIRNKTKGYTTEYNRSVKGRFTISRDNTQNMLYLQMNSLRPEDTATYYCARIGVNNGGSLDYWGQGVMVTVSS

SEQ ID NO: 16 - protein, 113 aa, synthetic construct
  REGION 1..113: note = Clone 14D3 VL
DILIIQSPASLTVSAGARVTMSCKSSQSLLYSENNQDYLAWYQQKPGQFPKLLIYGASNRHTGVPDRFTGSGSGTDFTLTISSVQAEDLADYYCEQTYSYPYTFGAGTKLELK

SEQ ID NO: 17 - protein, 8 aa, synthetic construct
  REGION 1..8: note = Clone 14D3 CDR1 of the heavy chain
GFTFTDFY

SEQ ID NO: 18 - protein, 10 aa, synthetic construct
  REGION 1..10: note = Clone 14D3 CDR2 of the heavy chain
IRNKTKGYTT

SEQ ID NO: 19 - protein, 15 aa, synthetic construct
  REGION 1..15: note = Clone 14D3 CDR3 of the heavy chain
ARIGVNNGGSLDYWG

SEQ ID NO: 20 - protein, 12 aa, synthetic construct
  REGION 1..12: note = Clone 14D3 CDR1 of the light chain
QSLLYSENNQDY

SEQ ID NO: 21 - skipped

SEQ ID NO: 22 - protein, 9 aa, synthetic construct
  REGION 1..9: note = Clone 14D3 CDR3 of the light chain
EQTYSYPYT

SEQ ID NO: 23 - protein, 122 aa, synthetic construct
  REGION 1..122: note = Clone 14D8 variable region of the heavy chain
EVKLLESGGGLVQPGGSMRLSCAASGFTFTDFYMNWIRQPAGKAPEWLGLIRNKANGYTTVYNPSVKGRFTISRDNTQNMLYLQMNTLRGEDTATYYCARIGINNGGSLDYWGQGVMVTVSS

SEQ ID NO: 24 - protein, 113 aa, synthetic construct
  REGION 1..113: note = Clone 14D8 variable region of the light chain
DILINQSPASLTVSTGEKVTMSCRSSQSLLYSEKNQDYLAWYQQKPGQFPKLLIYGASYRHTGVPDRFTGSGSGTDFTLTISSVQAEDLADYYCEQTYSYPYTFGAGTKLELK

SEQ ID NO: 25 - protein, 8 aa, synthetic construct
  REGION 1..8: note = Clone 14D8 CDR1 of the heavy chain; note = Clone 14D8 CDR1 of the heavy chain
GFTFTDFY

SEQ ID NO: 26 - protein, 10 aa, synthetic construct
  REGION 1..10: note = Clone 14D8 CDR2 of the heavy chain
IRNKANGYTT

SEQ ID NO: 27 - protein, 15 aa, synthetic construct
  REGION 1..15: note = Clone 14D8 CDR3 of the heavy chain
ARIGINNGGSLDYWG

SEQ ID NO: 28 - protein, 12 aa, synthetic construct
  REGION 1..12: note = Clone 14D8 CDR1 of the light chain
QSLLYSEKNQDY

SEQ ID NO: 29 - skipped

SEQ ID NO: 30 - protein, 9 aa, synthetic construct
  REGION 1..9: note = Clone 14D8 CDR3 of the light chain
EQTYSYPYT

SEQ ID NO: 31 - protein, 122 aa, synthetic construct
  REGION 1..122: note = Clone 7A12 variable region of the heavy chain
EVKLLESGGGLVQPGGSMRLSCAASGFTFTDFYMNWIRQPAGKAPEWLGLIRNKANGYTTQYNPSVKGRFTISRDNTQNMLYLQMNTLRGEDTATYYCARIGINNGGSLDYWGQGVMVTVSS

SEQ ID NO: 32 - protein, 113 aa, synthetic construct
  REGION 1..113: note = Clone 7A12 variable region of the light chain
DILINQSPASLTVSAGEKVTMSCKSSQSLLYSEKNQDYLAWYQQKPGQSPKLLMYGASYRHTGVPDRFTGSGSGTDFTLTISSVQAEDLADYYCEQTYSYPYTFGAGTKLELK

SEQ ID NO: 33 - protein, 8 aa, synthetic construct
  REGION 1..8: note = Clone 7A12 CDR1 of the heavy chain
GFTFTDFY

SEQ ID NO: 34 - protein, 10 aa, synthetic construct
  REGION 1..10: note = Clone 7A12 CDR2 of the heavy chain
IRNKANGYTT

SEQ ID NO: 35 - protein, 15 aa, unidentified
  REGION 1..15: note = Clone 7A12 CDR3 of the heavy chain
ARIGINNGGSLDYWG

SEQ ID NO: 36 - protein, 12 aa, synthetic construct
  REGION 1..12: note = Clone 7A12 CDR1 of the light chain
QSLLYSEKNQDY

SEQ ID NO: 37 - skipped

SEQ ID NO: 38 - protein, 9 aa, synthetic construct
  REGION 1..9: note = Clone 7A12 CDR3 of the light chain
EQTYSYPYT

SEQ ID NO: 39 - protein, 122 aa, synthetic construct
  REGION 1..122: note = Clone 8A11 variable region of the heavy chain
EVKLLESGGGLVQPGGSMRLSCAASGFTFTDFYMNWIRQPAGKAPEWLGLIRNKTKGYTTEYNTSVKGRFTISRDNTQNMLYLQMNSLRPEDTATYYCARIGVNNGGSLDYWGQGVMVTVSS

SEQ ID NO: 40 - protein, 113 aa, synthetic construct; identical to SEQ ID NO: 16
  REGION 1..113: note = Clone 8A11 variable region of the light chain

SEQ ID NO: 41 - protein, 8 aa, synthetic construct
  REGION 1..8: note = Clone 8A11 CDR1 of the heavy chain
GFTFTDFY

SEQ ID NO: 42 - protein, 10 aa, synthetic construct
  REGION 1..10: note = Clone 8A11 CDR2 of the heavy chain
IRNKTKGYTT

SEQ ID NO: 43 - protein, 15 aa, synthetic construct
  REGION 1..15: note = Clone 8A11 CDR3 of the heavy chain
ARIGVNNGGSLDYWG

SEQ ID NO: 44 - protein, 12 aa, synthetic construct
  REGION 1..12: note = Clone 8A11 CDR1 of the light chain
QSLLYSENNQDY

SEQ ID NO: 45 - skipped

SEQ ID NO: 46 - protein, 9 aa, synthetic construct
  REGION 1..9: note = Clone 8A11 CDR3 of the light chain
EQTYSYPYT

SEQ ID NO: 47 - protein, 122 aa, synthetic construct; identical to SEQ ID NO: 31
  REGION 1..122: note = Clone 21A3 variable region of the heavy chain

SEQ ID NO: 48 - protein, 113 aa, synthetic construct; identical to SEQ ID NO: 32
  REGION 1..113: note = Clone 21A3 variable region of the light chain

SEQ ID NO: 49 - protein, 8 aa, synthetic construct
  REGION 1..8: note = Clone 21A3 CDR1 of the heavy chain
GFTFTDFY

SEQ ID NO: 50 - protein, 10 aa, synthetic construct
  REGION 1..10: note = Clone 21A3 CDR2 of the heavy chain
IRNKANGYTT

SEQ ID NO: 51 - protein, 15 aa, synthetic construct
  REGION 1..15: note = Clone 21A3 CDR3 of the heavy chain
ARIGINNGGSLDYWG

SEQ ID NO: 52 - protein, 12 aa, synthetic construct
  REGION 1..12: note = Clone 21A3 CDR1 of the light chain
QSLLYSEKNQDY

SEQ ID NO: 53 - skipped

SEQ ID NO: 54 - protein, 9 aa, synthetic construct
  REGION 1..9: note = Clone 21A3 CDR3 of the light chain
EQTYSYPYT

SEQ ID NO: 55 - protein, 122 aa, synthetic construct
  REGION 1..122: note = Clone 10C3 variable region of the heavy chain
EVKLLESGGGLVQPGGSMRLSCAASGFTFTDFYMNWIRQPAGETPEWLGLIRNKTKGYTTEYNPSVKGRFTISRDNTQNMLYLQMNSLRPEDTATYYCARIGTNNGGSLDYWGQGVMVTVSS

SEQ ID NO: 56 - protein, 113 aa, synthetic construct; identical to SEQ ID NO: 16
  REGION 1..113: note = Clone 10C3 variable region of the light chain

SEQ ID NO: 57 - protein, 8 aa, synthetic construct
  REGION 1..8: note = Clone 10C3 CDR1 of the heavy chain
GFTFTDFY

SEQ ID NO: 58 - protein, 10 aa, synthetic construct
  REGION 1..10: note = Clone 10C3 CDR2 of the heavy chain
IRNKTKGYTT

SEQ ID NO: 59 - protein, 15 aa, synthetic construct
  REGION 1..15: note = Clone 10C3 CDR3 of the heavy chain
ARIGTNNGGSLDYWG

SEQ ID NO: 60 - protein, 12 aa, synthetic construct
  REGION 1..12: note = Clone 10C3 CDR1 of the light chain
QSLLYSENNQDY

SEQ ID NO: 61 - skipped

SEQ ID NO: 62 - protein, 9 aa, synthetic construct
  REGION 1..9: note = Clone 10C3 CDR3 of the light chain
EQTYSYPYT

SEQ ID NO: 63 - protein, 122 aa, synthetic construct
  REGION 1..122: note = Clone 18F9 variable region of the heavy chain
EVKLLESGGGLVQPGGSMRLSCVVSGFTFTDFYMNWIRQAAGKAPEWLGLIRNKVNGYRTEYNPSVKGRFTISRDNIQNMLYLQMNTLRAEDTATYYCARIGINNGGSLDYWGQGVMVTVSS

SEQ ID NO: 64 - protein, 113 aa, synthetic construct
  REGION 1..113: note = Clone 18F9 variable chain of the light region
DILINQSPASLTVSAGEKVTMSCKSSQSLLYSENNQDYLAWYQQKPGQFPKLLIYGASNRHTGVPDRFTGSGSGTDFTLTISSVQAEDLADYYCEQTYSYPYTFGAGTKLELK

SEQ ID NO: 65 - protein, 8 aa, synthetic construct
  REGION 1..8: note = Clone 18F9 CDR1 of the heavy chain
GFTFTDFY

SEQ ID NO: 66 - protein, 10 aa, synthetic construct
  REGION 1..10: note = Clone 18F9 CDR2 of the heavy chain
IRNKVNGYRT

SEQ ID NO: 67 - protein, 15 aa, synthetic construct
  REGION 1..15: note = Clone 18F9 CDR3 of the heavy chain
ARIGINNGGSLDYWG

SEQ ID NO: 68 - protein, 12 aa, synthetic construct
  REGION 1..12: note = Clone 18F9 CDR1 of the light chain
QSLLYSENNQDY

SEQ ID NO: 69 - skipped

SEQ ID NO: 70 - protein, 9 aa, synthetic construct
  REGION 1..9: note = Clone 18F9 CDR3 of the light chain
EQTYSYPYT

SEQ ID NO: 71 - protein, 122 aa, synthetic construct
  REGION 1..122: note = Clone 15C5 variable region of the heavy chain
EVKLLESGGGLVQPGGSMRLSCAASGFTFTDFYMNWIRQPAGKAPEWLGLIRNKAYGYTTEYNPSVKGRFTISRDNTQDMLYLQMNTLRAEDTATYYCARIGINYGGSLDYWGQGVMVTVSS

SEQ ID NO: 72 - protein, 113 aa, synthetic construct
  REGION 1..113: note = Clone 15C5 variable region of the light chain
DILINQSPASLTVSAGEKVTVSCKSSQSLLYSESNQDYLAWYQQKPGQFPKLLIYGASYRHTGVPDRFTGSGSGTDFTLTISSVQAEDLAHYYCEQTYSYPYTFGAGTKLELK

SEQ ID NO: 73 - protein, 8 aa, synthetic construct
  REGION 1..8: note = Clone 15C5 CDR1
GFTFTDFY

SEQ ID NO: 74 - protein, 10 aa, synthetic construct
  REGION 1..10: note = Clone 15C5 CDR2 of the heavy chain
IRNKAYGYTT

SEQ ID NO: 75 - protein, 15 aa, synthetic construct
  REGION 1..15: note = Clone 15C5 CDR3 of the heavy chain
ARIGINYGGSLDYWG

SEQ ID NO: 76 - protein, 12 aa, synthetic construct
  REGION 1..12: note = Clone 15C5 CDR1 of the light chain
QSLLYSESNQDY

SEQ ID NO: 77 - skipped

SEQ ID NO: 78 - protein, 9 aa, synthetic construct
  REGION 1..9: note = Clone 15C5 CDR3 of the light chain
EQTYSYPYT

SEQ ID NO: 79 - protein, 122 aa, synthetic construct
  REGION 1..122: note = Clone 1G6 variable region of the heavy chain
EVKLLESGGGLVQPGGSLRLSCVASGFTFTDFYMNWIRQPAGKAPEWLGLIRNKANGFTTEYNPSVKGRFTISRDNTQHMLYLQMNTLRAEDTATYYCARIGINNGGSLDYWGQGVMVTVSS

SEQ ID NO: 80 - protein, 113 aa, synthetic construct
  REGION 1..113: note = Clone 1G6 variable region of the light chain
DILINQSPASLTVSTGEKVTMSCKSSQSLLYSENKQDYLAWYQQKPGQFPKLLIYGASNRHTGVPDRFTGSGSGTDFTLTINIVQAEDLADYYCEQTYSYPYTFGAGTKLELK

SEQ ID NO: 81 - protein, 8 aa, synthetic construct
  REGION 1..8: note = Clone 1G6 CDR1 of the heavy chain
GFTFTDFY

SEQ ID NO: 82 - protein, 10 aa, synthetic construct
  REGION 1..10: note = Clone 1G6 CDR2 of the heavy chain
IRNKANGFTT

SEQ ID NO: 83 - protein, 15 aa, synthetic construct
  REGION 1..15: note = Clone 1G6 CDR3 of the heavy chain
ARIGINNGGSLDYWG

SEQ ID NO: 84 - protein, 12 aa, synthetic construct
  REGION 1..12: note = Clone 1G6 CDR1 of the light chain
QSLLYSENKQDY

SEQ ID NO: 85 - skipped

SEQ ID NO: 86 - protein, 9 aa, synthetic construct
  REGION 1..9: note = Clone 1G6 CDR3 of the light chain
EQTYSYPYT

SEQ ID NO: 87 - protein, 112 aa, synthetic construct
  REGION 1..112: note = Clone 13F4 VL
DVVMTQTPPSLSVAIGQSVSISCKSSQSLVYSDGKTYLHWLLQSPGRSPKRLIYQVSNLGSGVPDRFSGTGLQKDFTLKISRVEAEDLGVYYCAQTTHFPPTFGSGTKLEIK